(19) United States
(12) Patent Application Publication
(10) Pub. No.: US 2017/0312334 A1
(43) Pub. Date: Nov. 2, 2017

(54) METHODS AND COMPOSITIONS FOR TREATING INFLAMMATORY DISORDERS

(71) Applicant: ROGNE BIOSCIENCE INC., Portola Valley, CA (US)

(72) Inventor: Thomas Gadek, Portola Valley, CA (US)

(21) Appl. No.: 15/301,583

(22) PCT Filed: Apr. 2, 2015
(86) PCT No.: PCT/US15/23999

§ 371 Date: Oct. 3, 2016

Related U.S. Application Data
(60) Provisional application No. 61/974,335, filed on Apr. 2, 2014.

(51) Int. Cl. A61K 38/17; A61K 38/00; A61K 39/395; C07K 16/18; A61K 31/7088; A61K 45/00; A61K 45/06; C12N 15/113

(52) U.S. Cl. CPC A61K 38/1709; A61K 45/06; A61K 38/00; C12N 15/1136; C07K 16/18; A61K 31/7088; A61K 45/00; A61K 39/3955; C07K 2317/76; C12N 2320/31

(57) ABSTRACT

Disclosed herein, in certain embodiments, are methods and compositions for treating inflammatory disorders. In some embodiments, the methods comprise co-administering synergistic combinations of modulators of inflammation.

### CROSS-REFERENCE

This application claims the benefit of U.S. Provisional Patent Application No. 61/974,335 titled “Methods of Treating Inflammatory Disorders” and filed 2 Apr. 2014, which is incorporated herein by reference in its entirety.

### SUMMARY OF THE INVENTION

The present disclosure provides a method for controlling cellular expression of a gene, the method comprising contacting a cell with an effective amount of an agent that maintains NF-κB activity in the cell at a resting or baseline level such that expression of the gene is controlled.

The present disclosure provides a method for controlling cellular expression of a gene, the method comprising contacting a cell with an effective amount of an agent that inhibits an undesired increase in nuclear NF-κB activity such that expression of the gene controlled.

The present disclosure provides a method for controlling cellular expression of a gene, the method comprising contacting a cell with an effective amount of an agent that maintains NF-κB activity in the cell at a resting or baseline level such that expression of the gene is controlled.

The present disclosure provides a method for controlling cellular expression of a gene, the method comprising contacting a cell with an effective amount of an agent that inhibits an undesired increase in nuclear NF-κB activity such that expression of the gene is controlled.

The present disclosure provides a method for controlling cellular expression of a gene, the method of control comprising contacting a cell with an effective amount of an agent that antagonizes or regulates the formation of the PP2A holoenzyme such that expression of the gene is controlled.

The present disclosure provides a method for controlling cellular expression of a gene, the method of control comprising contacting a cell with an effective amount of an agent that stabilizes a complex of the PP2A core enzyme and a protein in the NF-κB pathway such that expression of the gene is controlled.

The present disclosure provides a method for controlling cellular expression of a gene, the method of control comprising contacting a cell with an effective amount of an agent that stabilizes a complex of the PP2A holoenzyme and proteins in the NF-κB pathway such that expression of the gene is controlled.

The present disclosure provides a method for controlling cellular expression of a gene encoding a cytokine, the method comprising contacting a cell with an effective amount of an agent that inhibits an undesired increase in NF-κB activity such that expression of the gene is controlled.

The present disclosure provides a method for controlling cellular expression of a gene encoding a cytokine, the method comprising contacting a cell with an effective amount of an agent that antagonizes the formation of the PP2A holoenzyme such that expression of the gene is controlled.

The present disclosure provides a method for controlling cellular expression of a gene encoding a cytokine, the method comprising contacting a cell with an effective amount of an agent that stabilizes a complex of the PP2A core enzyme and proteins in the NF-κB pathway such that expression of the gene is controlled.

The present disclosure provides a method for controlling cellular expression of a gene encoding a cytokine, the method comprising stabilizing a complex of the PP2A holoenzyme and proteins in the NF-κB pathway such that expression of the gene is controlled.

In some embodiments, the gene is selected from the group consisting of TNFα, IL-6, IL-12, IL-17, IL-23, and combinations thereof. In some embodiments, the gene is selected from the group consisting of IL-1, IL-2, IL-3, IL-4, IL-5, IL-6, IL-7, IL-8, IL-9, IL-11, IL-12, IL-13, IL-14, IL-15, IL-16, IL-17, IL-18, IL-19, IL-20, IL-21, IL-22, IL-23, IL-24, 11-25, IL-26, IL-27, IL-28, IL-29, IL-30, a TNF family member, an IFN family member, MCP-1, MIP-1, and any combination thereof.

In some embodiments, the cell is a eukaryotic cell.

In some embodiments, the contacting occurs in vivo.

In some embodiments, the agent is not a peptide derived from a chemerin protein.

In some embodiments, the agent is not derived from human C15 peptide. In some embodiments, the agent is not a peptide having the amino acid sequence AGEDPHSFYFPGQFA (SEQ ID NO: 1), or a peptide having at least about 50%, 60%, 70%, 80%, 90%, 95%, 99%, or 100% sequence identity thereto.

In some embodiments, the agent is not a human C15 peptide having the formula AGEDPHSFYFPGQFA (SEQ ID NO: 1).

In other embodiments, the agent is a human C15 peptide having the formula AGEDPHSFYFPGQFA (SEQ ID NO: 1).

In some embodiments, the agent binds to a protein of the NF-κB pathway.

In some embodiments, the agent regulates PP2A.

In some embodiments, the agent antagonizes the formation of the PP2A holoenzyme.

In some embodiments, the agent stabilizes a complex of the PP2A core enzyme and proteins in the NF-κB pathway.

In some embodiments, the agent stabilizes a complex of the PP2A holoenzyme and proteins in the NF-κB pathway. In some embodiments, the agent is a competitive antagonist of a human C15 peptide having the formula AGEDPHSFYFPGQFA (SEQ ID NO: 1). In some embodiments, the agent is a competitive antagonist of the binding of a human C15 peptide having the formula AGEDPHSFYFPGQFA (SEQ ID NO: 1) to PP2A. In some embodiments, the agent is a competitive antagonist of the binding of a human C15 peptide having the formula AGEDPHSFYFPGQFA (SEQ ID NO: 1) to one or more proteins in the NF-κB pathway.

In some embodiments, the agent is a competitive antagonist of a human PR70.

In some embodiments, the agent is a competitive antagonist of a human PR72.

In some embodiments, the agent is a competitive antagonist of the binding of a PP2A B subunit to PP2A (e.g., PP2A core enzyme). In some embodiments, the agent is a competitive antagonist of the binding of human PR70 to PP2A. In some embodiments, the agent is a competitive antagonist of the binding of human PR72 to PP2A. In some embodiments, the agent is a competitive antagonist of the binding of human PR70 to an A subunit of PP2A. In some embodiments, the agent is a competitive antagonist of the binding of human PR70 to one or more proteins in the NF-κB pathway. In some embodiments, the agent is a competitive antagonist of the binding of human PR70 to a PP2A core enzyme. In some embodiments, the agent inhibits formation of a PP2A holoenzyme complex. In some embodiments, the agent inhibits formation of a PP2A holoenzyme complex comprising PR70. In some embodiments, the agent does not directly interact with a C subunit of PP2A. In some embodiments, the agent does not directly interact with a C subunit of the PP2A core enzyme and/or a protein in the NF-κB pathway.

In some embodiments, the agent maintains or increases nuclear localization of PP2A.

In some embodiments, the agent maintains or increases an unphosphorylated state of a residue of NF-κB in the cell.

In some embodiments, the residue of NF-κB in an unphosphorylated state is Ser 536 of NF-κB p65. In some embodiments, the agent inhibits an undesired increase in the level of phosphorylation of Ser 536 relative to Ser 276 of NF-κB.

In some embodiments, the agent inhibits an undesired increase in the level of phosphorylation of IκB Kinase (IKK). In some embodiments, the agent inhibits an undesired increase in the level of phosphorylation of Ser 181 of IκB Kinase.

In some embodiments, the agent maintains a NF-κB in an inactive state.

In some embodiments, the NF-κB maintained in an inactive state is NF-κB p65. In some embodiments, the NF-κB maintained in an inactive state is bound to IκB.

In some embodiments, the agent binds to PR70. In some embodiments, the agent binds to PR72.

In some embodiments, the agent binds to an amino acid of an FYF (SEQ ID NO: 2) sequence of PR70. In some embodiments, the agent binds to amino acid Phe 128 of PR70. In some embodiments, the agent binds to a same region of PP2A as PR70. In some embodiments, the same region of PP2A comprises a PP2A region that binds to an amino acid of an FYF (SEQ ID NO: 2) sequence of PR70.

In some embodiments, the agent is a small molecule.

In some embodiments, the agent is an antibody.

In some embodiments, the agent is a nucleic acid.

In some embodiments, the nucleic acid is RNA. In some embodiments, the nucleic acid is DNA.

In some embodiments, the agent is not a peptide.

In some embodiments, the agent is a peptide.

In some embodiments, the peptide comprises the amino acid sequence FYF (SEQ ID NO: 2).

In some embodiments, the peptide comprises the amino acid sequence FYFP (SEQ ID NO: 3). In some embodiments, the peptide comprises the amino acid sequence PFYFP (SEQ ID NO: 4). In some embodiments, the peptide comprises the amino acid sequence PXFYFP (SEQ ID NO: 5), wherein X is any amino acid or analog thereof. In some embodiments, the peptide comprises the amino acid sequence P(S/T)FYFP (SEQ ID NO: 6). In some embodiments, the peptide comprises the amino acid sequence PSFYFP (SEQ ID NO: 7), wherein X is any amino acid or analog thereof. In some embodiments, the peptide comprises the amino acid sequence PTFYFP (SEQ ID NO: 8), wherein X is any amino acid or analog thereof. In some embodiments, the peptide comprises the amino acid sequence PX(S/T)FYFP (SEQ ID NO: 9), wherein X is any amino acid or analog thereof. In some embodiments, the peptide comprises the amino acid sequence PHSFYFP (SEQ ID NO: 10). In some embodiments, the peptide comprises the amino acid sequence PHTFYFP (SEQ ID NO: 11).

In some embodiments, the peptide comprises a nuclear translocation signal sequence. In some embodiments, the nuclear translocation signal sequence comprises one or more gapped dipeptides linked to nuclear localization. For example, in some embodiments, the nuclear translocation signal sequence comprises 1, 2, 3, 4, 5, 6, or 7 or more gapped dipeptides. In some embodiments, the nuclear translocation signal sequence comprises one or more nested and gapped dipeptides linked to nuclear localization. In some embodiments, a gap between a gapped dipeptide is 0, 1, 2, 3, 4, or 5 amino acids in length. In some embodiments, a gapped dipeptide is GP (SEQ ID NO: 12), DS (SEQ ID NO: 13), PS (SEQ ID NO: 14), PP (SEQ ID NO: 15), or PG (SEQ ID NO: 16). For example, in some embodiments, the nuclear translocation signal sequence comprises a gapped dipeptide GP with a gap bewteen the gapped dipeptide of 2 amino acids (i.e. GXXP (SEQ ID NO: 17)). For example, in some embodiments, the nuclear translocation signal sequence comprises a gapped dipeptide DS with a gap bewteen the gapped dipeptide of 2 amino acids (i.e. DXXS (SEQ ID NO: 18)). For example, in some embodiments, the nuclear translocation signal sequence comprises a gapped dipeptide PS with a gap bewteen the gapped dipeptide of 1 amino acid (i.e. PXS (SEQ ID NO: 19)). For example, in some embodiments, the nuclear translocation signal sequence comprises a gapped dipeptide PP with a gap bewteen the gapped dipeptide of 5 amino acids (i.e. PXXXXXP (SEQ ID NO: 20)). For example, in some embodiments, the nuclear translocation signal sequence comprises a gapped dipeptide PG with a gap bewteen the gapped dipeptide of 0 amino acids (i.e. PG (SEQ ID NO: 16)). In some embodiments, any one gapped dipeptide may be sufficient for nuclear localization. In some embodiments, 2, 3, 4, 5, 6, 7 or more gapped dipeptides are sufficient for nuclear localization.

In some embodiments, the nuclear translocation signal sequence comprises the sequence: GXXPXS (SEQ ID NO: 21). In some embodiments, the nuclear translocation signal sequence comprises the sequence: GXDPXS (SEQ ID NO: 22). In some embodiments, the nuclear translocation signal sequence comprises the sequence: GXXPXXXXXPG (SEQ ID NO: 23). In some embodiments, the nuclear translocation signal sequence comprises the sequence: GXXPXXXXXP (SEQ ID NO: 24). In some embodiments, the nuclear translocation signal sequence comprises the sequence: DPXS (SEQ ID NO: 25). In some embodiments, the nuclear translocation signal sequence comprises the sequence: DPXSXXXP (SEQ ID NO: 26). In some embodiments, the nuclear translocation signal sequence comprises the sequence: DXXSXXXPG (SEQ ID NO: 27). In some embodiments, the nuclear translocation signal sequence comprises the sequence: PXSXXXP (SEQ ID NO: 28). In some embodiments, the nuclear translocation signal sequence comprises the sequence: PXSXXXPG (SEQ ID NO: 29). In some embodiments, the nuclear translocation signal sequence comprises the sequence: GxDPxSXXXPG (SEQ ID NO: 30). In some embodiments, the nuclear translocation signal sequence comprises the sequence: XGXDPXSXXXPGXXX (SEQ ID NO: 31).

In some embodiments, the agent enters the cell independently of ChemR23.

In some embodiments, entry of the agent into the cell depends on ChemR23.

The present disclosure provides a method for controlling nuclear translocation of NF-κB in a cell comprising contacting the cell with an agent that: (a) controls the level of PP2A associated with NF-κB, (b) controls the level of PP2A associated with IκB Kinase, (c) controls the level of PP2A core enzyme associated with NF-κB, (d) controls the level of PP2A core enzyme associated with IκB Kinase, (e) controls the level of PP2A holoenzyme associated with NF-κB, (f) controls the level of PP2A holoenzyme associated with IκB Kinase, (g) prevents the association of the regulatory B subunit of PP2A with the core enzyme of PP2A, (h) inhibits an undesired increase in the level of phosphorylation of Ser 536 relative to Ser 276 of NF-κB, (i) inhibits an undesired increase in the level of phosphorylation of Ser 181 of IκB Kinase, or any combination thereof.

In some embodiments, the cell is stimulated with Chemerin, IL-1, TNFα, IFNγ, LPS, R848, BAFF, LTβ, CD40L, T-cell antigen, B-cell antigen, Zymosan, or any combination thereof. In some embodiments, the cell is stimulated via a Growth Factor Receptors, a TNF Receptor, an IL-1 Receptor, a Toll-like Receptor, a T-cell Receptor, a B-cell Receptor, BAFF Receptor, LTβ Receptor, CD40, ChemR23, GPR-1, or any combination thereof. In some embodiments, the NF-κB pathway of the cell has been stimulated.

The present disclosure provides a method of antagonizing the formation of PP2A holoenzyme in an individual in need thereof, comprising administering to the individual an agent.

The present disclosure provides a method of antagonizing the formation of PP2A holoenzyme in an individual in need thereof, comprising administering to the individual a competitive antagonist of a human C15 peptide having the formula AGEDPHSFYFPGQFA (SEQ ID NO: 1).

The present disclosure provides a method of stabilizing the PP2A core enzyme complexed with proteins of the NF-κB pathway in an individual in need thereof comprising administering to the individual an agent, or a peptide with at least 80% sequence identity thereto.

The present disclosure provides a method of stabilizing the PP2A core enzyme complexed with proteins of the NF-κB pathway in an individual in need thereof comprising administering to the individual a competitive antagonist of a human C15 peptide having the formula AGEDPHSFYFPGQFA (SEQ ID NO: 1).

In some embodiments, the method further comprises decreasing secretion of a cytokine by the cell. In some embodiments, the method further comprises decreasing secretion of TNFα, IL-6, IL-12, IL-17, IL-23, or any combination thereof by the cell. In some embodiments, the method further comprises decreasing secretion of IL-1, IL-2, IL-3, IL-4, IL-5, IL-6, IL-7, IL-8, IL-9, IL-11, IL-12, IL-13, IL-14, IL-15, IL-16, IL-17, IL-18, IL-19, IL-20, IL-21, IL-22, IL-23, IL-24, 11-25, IL-26, IL-27, IL-28, IL-29, IL-30, or any combination thereof by the cell, and decreasing secretion of a TNF family member, an IFN family member, MCP-1, MIP-1, or any combination thereof by the cell.

In some embodiments, the individual suffers from an inflammatory disorder.

In some embodiments, the inflammatory disorder is responsive to treatment with a glucocortico steroid. In some embodiments, the inflammatory disorder is responsive to treatment with dexamethasone. In some embodiments, the inflammatory disorder is selected from the group consisting of Psoriasis, Atopic Dermatitis, Contact Dermatitis, Lichen Planus, Acne, Alopecia Areata, IBD, Crohn's Disease and/or Ulcerative Colitis, Uveitis, Dry Eye, Blepharitis, Allergic conjunctivitis, Iritis, a retinal inflammatory disease, and any combintion thereof. In some embodiments, the inflammatory disorder is a retinal inflammatory disease that is AMD. In some embodiments, the inflammatory disorder is a retinal inflammatory disease that is DME. In some embodiments, the inflammatory disorder is selected from the group consisting of Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barre syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behcet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease; abdominal aortic aneurysm (AAA); and any combination thereof.

The present disclosure provides a method of antagonizing the formation of PP2A holoenzyme in an individual in need thereof, comprising administering to the individual an effective amount of and agent, wherein the peptide binds to PP2A.

The present disclosure provides a method of antagonizing the formation of PP2A holoenzyme in an individual in need thereof, comprising administering to the individual an effective amount of an agent, wherein the agent is a competitive antagonist of the binding of a human C15 peptide having the formula AGEDPHSFYFPGQFA (SEQ ID NO: 1) to PP2A.

The present disclosure provides a method of stabilizing PP2A core enzyme complexed with proteins of the NF-κB pathway in an individual in need thereof comprising administering to the individual an effective amount of an agent, wherein the agent binds to PP2A.

The present disclosure provides a method of stabilizing the PP2A core enzyme complexed with proteins of the NF-κB pathway in an individual in need thereof comprising administering to the individual an effective amount of an agent, wherein the agent is a competitive antagonist of the binding of a human C15 peptide having the formula AGEDPHSFYFPGQFA (SEQ ID NO: 1) to PP2A.

The present disclosure provides a method of increasing nuclear localization of PP2A core enzyme complexed with proteins of the NF-κB pathway in an individual in need thereof comprising administering to the individual an effective amount of an agent, wherein the agent binds to PP2A and increases the nuclear localization of the PP2A core enzyme.

The present disclosure provides a method of increasing nuclear localization of PP2A core enzyme complexed with proteins of the NF-κB pathway in an individual in need thereof comprising administering to the individual an effective amount of an agent, wherein the agent is a competitive antagonist of the binding of a human C15 peptide having the formula AGEDPHSFYFPGQFA (SEQ ID NO: 1) to PP2A and increases the nuclear localization of the PP2A core enzyme.

The present disclosure provides a method of treating an inflammatory disorder in an individual in need thereof, comprising administering to the individual an effective amount of an agent, or a peptide with at least 80% sequence identity thereto.

The present disclosure provides a method of treating an inflammatory disorder in an individual in need thereof, comprising administering to the individual an effective amount of an agent, wherein the agent is a competitive antagonist of the binding of a human C15 peptide having the formula AGEDPHSFYFPGQFA (SEQ ID NO: 1).

The present disclosure provides a method of antagonizing the formation of PP2A holoenzyme in an individual in need thereof, comprising administering to the individual an effective amount of an agent, wherein the agent is a competitive antagonist of the binding of human PR70.

The present disclosure provides a method of stabilizing the PP2A core enzyme complexed with proteins of the NF-κB pathway in an individual in need thereof comprising administering to the individual an effective amount of an agent, wherein the agent is a competitive antagonist of the binding of human PR70.

The present disclosure provides a method of treating an inflammatory disorder in an individual in need thereof, comprising administering to the individual an effective amount of an agent, wherein the agent is a competitive antagonist of the binding of human PR70.

The present disclosure provides a method of antagonizing the formation of PP2A holoenzyme in an individual in need thereof, comprising administering to the individual an effective amount of an agent, wherein the agent is a competitive antagonist of the binding of human PR70 to PP2A.

The present disclosure provides a method of stabilizing the PP2A core enzyme complexed with proteins of the NF-κB pathway in an individual in need thereof comprising administering to the individual an effective amount of an agent, wherein the agent is a competitive antagonist of the binding of human PR70 to PP2A.

The present disclosure provides a method of treating an inflammatory disorder in an individual in need thereof, comprising administering to the individual an effective amount of an agent, wherein the agent is a competitive antagonist of the binding of human PR70 to PP2A.

In some embodiments, the method further comprises administering an anti-inflammatory agent.

In some embodiments, the anti-inflammatory agent is selected from the group consisting of an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent an alkylating agent, an anti-metabolite, a plant alkaloid, a terpenoids, a topoisomerase inhibitor, an antitumor antibiotic, an antibody, a hormonal therapy, an anti-diabetes agent, a leukotriene inhibitor, and any combination thereof. In some embodiments, the anti-inflammatory agent is selected from the group consisting of alefacept, efalizumab, methotrexate, acitretin, isotretinoin, hydroxyurea, mycophenolate mofetil, sulfasalazine, 6-thioguanine, Dovonex, Taclonex, betamethasone, tazarotene, hydroxychloroquine, etanercept, adalimumab, infliximab, abatacept, rituximab, tratuzumab, AHN-12, Iodine-131 Anti-B1 antibody, anti-CD66 monoclonal antibody BW 250/183, anti-CD45 monoclonal antibody, antibody anti-anb3 integrin, BIW-8962, antibody BC8, antibody muJ591, indium In 111, monoclonal antibody MN-14, yttrium Y 90 monoclonal antibody MN-14, F105 monoclonal antibody, monoclonal antibody RAV12, CAT-192, antibody 3F8, 177Lu-J591, TB-403, anakinra, azathioprine, cyclophosphamide, cyclosporine A, leflunomide, d-penicillamine, amitriptyline, or nortriptyline, chlorambucil, nitrogen mustard, prasterone, LJP 394, LJP 1082, eculizumab, belibumab, rhuCD40L, epratuzumab, sirolimus, tacrolimus, pimecrolimus, thalidomide, antithymocyte globulin-equine, antithymocyte globulin-rabbit, Muromonab-CD3, basiliximab, daclizumab, riluzole, cladribine, natalizumab, interferon beta-lb, interferon beta-la, tizanidine, baclofen, mesalazine, asacol, pentasa, mesalamine, balsalazide, olsalazine, 6-mercaptopurine, AIN457, theophylline, D2E7, Mepolizumab, Canakinumab, Daclizumab, CNTO 328, ACZ885, CNTO 1275, (3S)—N-hydroxy-4-({4-[(4-hydroxy-2-butynyl)oxy]phenyl}sulfonyl)-2,2-dimet-hyl-3-thiomorpholine carboxamide, golimumab, Onercept, BG9924, Certolizumab Pegol, AZD9056, AZD5069, AZD9668, AZD7928, AZD2914, AZD6067, AZD3342, AZD8309, [(1R)-3-methyl-1-({(2S)-3-phenyl-2-[(pyrazin-2-ylcarbonyl)amino]propanoyl}amino)butyl]boronic acid, AMG-714, ABT-874, Tocilizumab, CAT-354, aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502, JTE-522, L-745,337, NS398, betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Pioglitazone, Rosiglitazone, Glimepiride, Glyburide, Chlorpropamide, Glipizide, Tolbutamide, Tolazamide, Glucophage, Metformin, glyburide+metformin, Rosiglitazone+metformin, Rosiglitazone+glimepiride, Exenatide, Insulin, Sitagliptin, glipizide+metformin, Repaglinide, Acarbose, Nateglinide, Orlistat, cisplatin; carboplatin; oxaliplatin; mechlorethamine; cyclophosphamide; chlorambucil; vincristine; vinblastine; vinorelbine; vindesine; mercaptopurine; fludarabine; pentostatin; cladribine; 5-fluorouracil (5FU); floxuridine (FUDR); cytosine arabinoside; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; finasteride; goserelin; aminoglutethimide; anastrozole; letrozole; vorozole; exemestane; 4-androstene-3,6,17-trione (“6-OXO”); 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl) indole-2-yl)-2,2-dimethylpropionaldehyde oxime-O-2-acetic acid; AME103; AME803; atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031; DG-051; MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimehtylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2methyl-1-oxopropyl-L-cycteine); SC-56938 (ethyl-1-[2-[4-(phenylmethyl)phenoxy]ethyl]-4-piperidine-carboxylate); VIA-2291; WY-47,288 (2-[(1-naphthalenyloxy) methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); busulphan; alemtuzumab; belatacept; posaconazole; fingolimod; an anti-CD40 ligand antibody, BG 9588; CTLA4Ig; LJP 394; an anti-IL10 antibody; an anti-CD20 antibody, rituximab; an anti-C5 antibody, eculizumab; doxycycline; or combinations thereof.

In some embodiments, the anti-inflammatory agent is administered before, after, or simultaneously with the modulator of inflammation.

In some embodiments, the agent is a competitive antagonist of the binding of the human C15 peptide (AGEDPHSFYFPGQFA (SEQ ID NO: 1)) to proteins in the NF-κB pathway and reduces the level of inflammatory cytokines in vitro or in vivo

The present disclosure provides an agent (i.e., a compound or active agent), wherein the agent is a competitive antagonist of a human C15 peptide having the formula AGEDPHSFYFPGQFA (SEQ ID NO: 1) and binds to a protein of the NF-κB pathway.

The present disclosure provides an agent, wherein the agent is a competitive antagonist of human PR70.

The present disclosure provides an agent, wherein the agent is a competitive antagonist of human PR70 and binds to a protein of the NF-κB pathway.

The present disclosure provides an agent, wherein the agent is a competitive antagonist of human PR70 and binds to a PP2A.

The present disclosure provides an agent wherein the agent has an amino acid sequence with at least 80%, 85%, 90%, 95%, or 100% sequence identity to LSINIPRFYFPEGLP (SEQ ID NO: 32), LSINIPRXFYFPEGLP (SEQ ID NO: 33), or LSINIPXRFYFPEGLP (SEQ ID NO: 34). In some embodiments, the agent is not a human C15 peptide having the formula AGEDPHSFYFPGQFA (SEQ ID NO: 1). In other embodiments, the agent is a human C15 peptide having the formula AGEDPHSFYFPGQFA (SEQ ID NO: 1).

The present disclosure provides an agent wherein the agent has an amino acid sequence with at least 80%, 85%, 90%, 95%, or 100% sequence identity to TSQSIPTFYFPRGRP (SEQ ID NO: 35), TSQSIPXTFYFPRGRP (SEQ ID NO: 36), or TSQSIPTXFYFPRGRP (SEQ ID NO: 37). In some embodiments, the agent is not a human C15 peptide having the formula AGEDPHSFYFPGQFA (SEQ ID NO: 1). In other embodiments, the agent is a human C15 peptide having the formula AGEDPHSFYFPGQFA (SEQ ID NO: 1).

The present disclosure provides an agent wherein the agent has an amino acid sequence with at least 80%, 85%, 90%, 95%, or 100% sequence identity to human PR70. In some embodiments, the agent is not a human C15 peptide having the formula AGEDPHSFYFPGQFA (SEQ ID NO: 1). In other embodiments, the agent is a human C15 peptide having the formula AGEDPHSFYFPGQFA (SEQ ID NO: 1).

In some embodiments, the agent is selected from the group consisting of: AGEDPHGYFLPGQFA (SEQ ID NO: 38), AGEDPHSFYFPGQFA (SEQ ID NO: 1), AGEDPHSFYFPGQFAF, AGEDPHSFYFPGQFAFS, PHSFYFPGQFA (SEQ ID NO: 41), XGEDPHSFYFPGQFA (SEQ ID NO: 42), AXEDPHSFYFPGQFA (SEQ ID NO: 43), AGXDPHSFYFPGQFA (SEQ ID NO: 44), AGEXPHSFYFPGQFA (SEQ ID NO: 45), AGEDXHSFYFPGQFA (SEQ ID NO: 46), AGEDPXSFYFPGQFA (SEQ ID NO: 47), AGEDPHXFYFPGQFA (SEQ ID NO: 48), AGEDPHSXYFPGQFA (SEQ ID NO: 49), AGEDPHSFXFPGQFA (SEQ ID NO: 50), AGEDPHSFYXPGQFA (SEQ ID NO: 51), AGEDPHSFYFXGQFA (SEQ ID NO: 52), AGEDPHSFYFPXQFA (SEQ ID NO: 53), AGEDPHSFYFPGXFA (SEQ ID NO: 54), AGEDPHSFYFPGQXA (SEQ ID NO: 55), AGEDPHSFYFPGQFX (SEQ ID NO: 56), AGEDPHSXYX′PGQFA (SEQ ID NO: 57), AGEDPHSXX′X″PGQFA (SEQ ID NO: 58), UGEDPHSFYFPGQFA (SEQ ID NO: 59), AUEDPHSFYFPGQFA (SEQ ID NO: 60), AGUDPHSFYFPGQFA (SEQ ID NO: 61), AGEUPHSFYFPGQFA (SEQ ID NO: 62), AGEDUHSFYFPGQFA (SEQ ID NO: 63), AGEDPUSFYFPGQFA (SEQ ID NO: 64), AGEDPHUFYFPGQFA (SEQ ID NO: 65), AGEDPHSUYFPGQFA (SEQ ID NO: 66), AGEDPHSFUFPGQFA (SEQ ID NO: 67), AGEDPHSFYUPGQFA (SEQ ID NO: 68), AGEDPHSFYFUGQFA (SEQ ID NO: 69), AGEDPHSFYFPUQFA (SEQ ID NO: 70), AGEDPHSFYFPGUFA (SEQ ID NO: 71), AGEDPHSFYFPGQUA (SEQ ID NO: 72), AGEDPHSFYFPGQFU (SEQ ID NO: 73), AGEDPHSUYU′PGQFA (SEQ ID NO: 74), AGEDPHSUU′U″PGQFA (SEQ ID NO: 75), and any combination thereof, wherein X is an unnatural amino acid or a natural amino acid or is absent, wherein X, X′ and X″ are same or different, wherein U is an unnatural amino acid, and wherein U, U′ and U″ are same or different. In some embodiments, a U is homo-serine. In some embodiments, a U is p-chlorophenylalanine.

In some embodiments, the agent does not consist of any one of the peptides selected from the group consisting of PHGYFLPGQPA (SEQ ID NO: 76); PHGYFLPGQFAF (SEQ ID NO: 77); PHGYFLPGQFAFS (SEQ ID NO: 78); AGEDPHGYFLPGQFA (SEQ ID NO: 38); AGEDPHGYFLPGQFAF (SEQ ID NO: 39); AGEDPHGYFLPGQFAFS (SEQ ID NO: 40); DPHGYFLPGQFA (SEQ ID NO: 81); EDPHGYFLPGQFA (SEQ ID NO: 82); GEDPHGYPLPGQFA (SEQ ID NO: 83); DPHGYFLPGQFAF (SEQ ID NO: 84); EDPHGYFLPGQFAF (SEQ ID NO: 85); GEDPHGYFLPGQFAF (SEQ ID NO: 86); DPHGYFLPGQFAFS (SEQ ID NO: 87); EDPHGYFLPGQFAFS (SEQ ID NO: 88); GEDPHGYFLPGQFAFS (SEQ ID NO: 89); PHSEYFPGQFA (SEQ ID NO: 90); PHSFYFPGQFAF (SEQ ID NO: 91); PHSFYFPGQFAFS (SEQ ID NO: 92); AGEDPHSFYFPGQFA (SEQ ID NO: 1); AGEDPHSFYPPGQFAF (SEQ ID NO: 93); AGEDPHSFYFPGQFAFS; DPHSFYFPGQFA (SEQ ID NO: 94); EDPHSFYFPGQFA (SEQ ID NO: 95); GEDPHSPYFPGQFA (SEQ ID NO: 96); DPHSFYPPGQFAF (SEQ ID NO: 97); EDPHSFYFPGQFAF (SEQ ID NO: 98); GEDPHSFYFPGQFAF (SEQ ID NO: 99); DPHSFYFPGQFAPS (SEQ ID NO: 100); EDPHSFYFPGQFAFS (SEQ ID NO: 101); GEDPHSFYFPGQFAFS (SEQ ID NO: 102); AQAGEDPHGYFLPGQFAFS (SEQ ID NO: 103); and QRAGEDPHSFYFPGQFAFS (SEQ ID NO: 104). In some embodiments, the agent has less than about 30%, 40%, 50%, 60%, 70%, 80%, 90% 95%, or 99% sequence identity with one or more of the peptides selected from the group consisting of PHGYFLPGQPA (SEQ ID NO: 76); PHGYFLPGQFAF (SEQ ID NO: 77); PHGYFLPGQFAFS (SEQ ID NO: 78); AGEDPHGYFLPGQFA (SEQ ID NO: 38); AGEDPHGYFLPGQFAF (SEQ ID NO: 39); AGEDPHGYFLPGQFAFS (SEQ ID NO: 40); DPHGYFLPGQFA (SEQ ID NO: 81); EDPHGYFLPGQFA (SEQ ID NO: 82); GEDPHGYPLPGQFA (SEQ ID NO: 83); DPHGYFLPGQFAF (SEQ ID NO: 84); EDPHGYFLPGQFAF (SEQ ID NO: 85); GEDPHGYFLPGQFAF (SEQ ID NO: 86); DPHGYFLPGQFAFS (SEQ ID NO: 87); EDPHGYFLPGQFAFS (SEQ ID NO: 88); GEDPHGYFLPGQFAFS (SEQ ID NO: 89); PHSEYFPGQFA (SEQ ID NO: 90); PHSFYFPGQFAF (SEQ ID NO: 91); PHSFYFPGQFAFS (SEQ ID NO: 92); AGEDPHSFYFPGQFA (SEQ ID NO: 1); AGEDPHSFYPPGQFAF (SEQ ID NO: 93); AGEDPHSFYFPGQFAFS; DPHSFYFPGQFA (SEQ ID NO: 94); EDPHSFYFPGQFA (SEQ ID NO: 95); GEDPHSPYFPGQFA (SEQ ID NO: 96); DPHSFYPPGQFAF (SEQ ID NO: 97); EDPHSFYFPGQFAF (SEQ ID NO: 98); GEDPHSFYFPGQFAF (SEQ ID NO: 99); DPHSFYFPGQFAPS (SEQ ID NO: 100); EDPHSFYFPGQFAFS (SEQ ID NO: 101); GEDPHSFYFPGQFAFS (SEQ ID NO: 102); AQAGEDPHGYFLPGQFAFS (SEQ ID NO: 103); and QRAGEDPHSFYFPGQFAFS (SEQ ID NO: 104). In some embodiments, the agent has less than about 80%, 85%, 90%, 91%, 92%, 93%, 94%, 95%, 96%, 97%, 98%, 99%, 99.5%, 99.6%, 99.7%, 99.8%, or 99.9% sequence identity to a naturally occurring chemerin C15 peptide. In some embodiments, the agent has less than about 80%, 85%, 90%, 91%, 92%, 93%, 94%, 95%, 96%, 97%, 98%, 99%, 99.5%, 99.6%, 99.7%, 99.8%, or 99.9% sequence identity to a human C15 peptide having the formula AGEDPHSFYFPGQFA (SEQ ID NO: 1).

In other embodiments, the agent has at least about 80%, 85%, 90%, 91%, 92%, 93%, 94%, 95%, 96%, 97%, 98%, 99%, 99.5%, 99.6%, 99.7%, 99.8%, or 99.9% sequence identity to a human C15 peptide having the formula AGEDPHSFYFPGQFA (SEQ ID NO: 1).

In some embodiments, the agent is a chimeric sequence comprising a human chemerin sequence and a sequence from a non-human organism.

In some embodiments, the agent binds to PR70.

In some embodiments, the agent binds to PR70 with an affinity of less than 10 nM, 1 nM, 100 pM, 10 pM, or 1 pM or less. In some embodiments, the agent binds to an amino acid of an FYF (SEQ ID NO: 2) sequence of PR70. In some embodiments, the agent binds to amino acid Phe 128 of PR70.

In some embodiments, the agent binds to a same region of PP2A as PR70. In some embodiments, the same region of PP2A comprises a PP2A region that binds to an amino acid of an FYF (SEQ ID NO: 2) sequence of PR70.

In some embodiments, the agent is a small molecule.

In some embodiments, the agent is an antibody.

In some embodiments, the agent is a nucleic acid. In some embodiments, the nucleic acid is RNA. In some embodiments, the nucleic acid is DNA.

In some embodiments, the agent is not a peptide.

In some embodiments, the agent is a peptide. In some embodiments, the peptide comprises the amino acid sequence FYF (SEQ ID NO: 2). In some embodiments, the peptide comprises the amino acid sequence FYFP (SEQ ID NO: 3). In some embodiments, the peptide comprises the amino acid sequence PFYFP (SEQ ID NO: 4). In some embodiments, the peptide comprises the amino acid sequence PXFYFP (SEQ ID NO: 5), wherein X is any amino acid or analog thereof. In some embodiments, the peptide comprises the amino acid sequence PSFYFP (SEQ ID NO: 7), wherein X is any amino acid or analog thereof. In some embodiments, the peptide comprises the amino acid sequence PTFYFP (SEQ ID NO: 8), wherein X is any amino acid or analog thereof. In some embodiments, the peptide comprises the amino acid sequence PXXFYFP (SEQ ID NO: 9), wherein X is any amino acid or analog thereof. In some embodiments, the peptide comprises the amino acid sequence PXSFYFP (SEQ ID NO: 9), wherein X is any amino acid or analog thereof. In some embodiments, the peptide comprises the amino acid sequence PXTFYFP (SEQ ID NO: 9), wherein X is any amino acid or analog thereof. In some embodiments, the peptide comprises the amino acid sequence PHSFYFP (SEQ ID NO: 10), wherein X is any amino acid or analog thereof. In some embodiments, the peptide comprises the amino acid sequence PHTFYFP (SEQ ID NO: 11), wherein X is any amino acid or analog thereof.

In some embodiments, the peptide comprises a nuclear translocation signal sequence. In some embodiments, the nuclear translocation signal sequence comprises one or more gapped dipeptides linked to nuclear localization. For example, in some embodiments, the nuclear translocation signal sequence comprises 1, 2, 3, 4, 5, 6, or 7 or more gapped dipeptides. In some embodiments, the nuclear translocation signal sequence comprises one or more nested and gapped dipeptides linked to nucelar localization. In some embodiments, a gap bewteen a gapped dipeptide is 0, 1, 2, 3, 4, or 5 amino acids in length. In some embodiments, a gapped dipeptide is GP (SEQ ID NO: 12), DS (SEQ ID NO: 13), PS (SEQ ID NO: 14), PP (SEQ ID NO: 15), or PG (SEQ ID NO: 16). For example, in some embodiments, the nuclear translocation signal sequence comprises a gapped dipeptide GP (SEQ ID NO: 12) with a gap bewteen the gapped dipeptide of 2 amino acids (i.e. GXXP (SEQ ID NO: 17)). For example, in some embodiments, the nuclear translocation signal sequence comprises a gapped dipeptide DS with a gap bewteen the gapped dipeptide of 2 amino acids (i.e. DXXS (SEQ ID NO: 18)). For example, in some embodiments, the nuclear translocation signal sequence comprises a gapped dipeptide PS with a gap bewteen the gapped dipeptide of 1 amino acid (i.e. PXS (SEQ ID NO: 19)). For example, in some embodiments, the nuclear translocation signal sequence comprises a gapped dipeptide PP with a gap bewteen the gapped dipeptide of 5 amino acids (i.e. PXXXXXP (SEQ ID NO: 20)). For example, in some embodiments, the nuclear translocation signal sequence comprises a gapped dipeptide PG with a gap bewteen the gapped dipeptide of 0 amino acids (i.e. PG (SEQ ID NO: 16)). In some embodiments, any one gapped dipeptide may be sufficient for nuclear localization. In some embodiments, 2, 3, 4, 5, 6, 7 or more gapped dipeptides are sufficient for nuclear localization.

In some embodiments, the nuclear translocation signal sequence comprises the sequence: GXXPXS (SEQ ID NO: 21). In some embodiments, the nuclear translocation signal sequence comprises the sequence: GXDPXS (SEQ ID NO: 22). In some embodiments, the nuclear translocation signal sequence comprises the sequence: GXXPXXXXXPG (SEQ ID NO: 23). In some embodiments, the nuclear translocation signal sequence comprises the sequence: GXXPXXXXXP (SEQ ID NO: 24).

In some embodiments, the nuclear translocation signal sequence comprises the sequence: DPXS (SEQ ID NO: 25). In some embodiments, the nuclear translocation signal sequence comprises the sequence: DPXSXXXP (SEQ ID NO: 26). In some embodiments, the nuclear translocation signal sequence comprises the sequence: DXXSXXXPG (SEQ ID NO: 27). In some embodiments, the nuclear translocation signal sequence comprises the sequence: PXSXXXP (SEQ ID NO: 28). In some embodiments, the nuclear translocation signal sequence comprises the sequence: PXSXXXPG (SEQ ID NO: 29). In some embodiments, the nuclear translocation signal sequence comprises the sequence: GXDPXSXXXPG (SEQ ID NO: 30). In some embodiments, the nuclear translocation signal sequence comprises the sequence: XGXDPXSXXXPGXXX (SEQ ID NO: 31).

In some embodiments, the agent consists of 6, 7, 8, 9, 10, 11, 12, 13, 14, 15, 16, 17, 18, 19, 20, 21, 22, 23, 24, 25, 26, 27, 28, 29, or 30 amino acids.

In some embodiments, the agent suppresses NF-κB p65 to a level similar to the suppression by a corticosteroid, wherein the agent is at least about 2, 3, 4, 5, 6, 7, 8, 9, 10, 20, 40, 50, 60, 80, 90, 100, 150, 200, 300, 400, or 500, or more times potent than the corticosteroid.

In some embodiments, the agent further comprises a detectable label.

### INCORPORATION BY REFERENCE

All publications, patents, and patent applications mentioned in this specification are herein incorporated by reference in their entirety, to the same extent as if each individual publication, patent, or patent application was specifically and individually indicated to be incorporated by reference.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 exemplifies PP2A holoenzymes. The catalytic subunit is bound to the scaffolding subunit to form the core dimer of PP2A. The core dimer can interact with a variety of regulatory subunits (R) to generate a diversity of forms. The regulatory subunits target the enzyme to specific substrates and subcellular regions.

FIG. 2A exemplifies a space filling model of the PP2A holoenzyme.

FIG. 2B exemplifies a space filling representation of the crystal structure of the PP2A holoenzyme complexed to a PR70 (B subunit) peptide including an expanded view of the PR70 FYF (SEQ ID NO: 2) motif.

FIG. 3A exemplifies an amino acid sequence alignment of PP2A regulatory subunit By to seven WD repeats and component β-strands from Pfam (SEQ ID NOs: 232 to 238).

FIG. 3B exemplifies a schematic of β-strand arrangement of the β-propeller fold of PP2A regulatory subunit By.

FIG. 3C exemplifies a schematic of the of PP2A regulatory subunit By model based on the Gβ1 crystal structure.

FIG. 4 exemplifies an amino acid sequence alignment of PP2A regulatory subunit families. (SEQ ID NOs: 239 to 249).

FIG. 5A exemplifies an amino acid sequence alignment of PP2A regulatory subunits (SEQ ID NOs: 250 to 251).

FIG. 5B exemplifies an amino acid sequence alignment of the human PP2A regulatory subunit PR70 and mutants in the FYF (SEQ ID NO: 2) motif that abolish binding to PP2A core enzyme (SEQ ID NOs: 252 to 257).

FIG. 5C exemplifies a Western-blot of immunoprecipitations using a flag antibody from cells expressing the indicated flag-PR70 constructs (from FIG. 5B). An SDS-PAGE gel was transferred to a PVDF membrane and probed with antibodies that recognize the flag tag, PP2A subunit A, and PP2A subunit C.

FIG. 6A exemplifies a Western-blot of immunoprecipitations using an IKKβ antibody from cells expressing the indicated flag-tagged constructs. An SDS-PAGE gel was transferred to a PVDF membrane and probed with antibodies that recognize IKKβ and IKKβ Ser181 phosphorylation.

FIG. 6B exemplifies a Western-blot of immunoprecipitations using an NF-κB p65 antibody from cells expressing the indicated flag-tagged constructs. An SDS-PAGE gel was transferred to a PVDF membrane and probed with antibodies that recognize NF-κB p65, NF-κB p65 Ser536 phosphorylation, and NF-κB p65 Ser276 phosphorylation.

FIG. 7 exemplifies a schematic of the G-protein- and arrestin-mediated signaling by the dopamine D₂ receptor.

FIG. 8 exemplifies a schematic of NF-κB mediated signaling.

FIG. 9 exemplifies a schematic of NF-κB mediated IL-23 expression.

FIG. 10A is a graph of inhibition of IL-1β secretion by the indicated agents. Agent #7 is human C15 peptide having the formula AGEDPHSFYFPGQFA (SEQ ID NO: 1) and was used at a concentration of 1 pM.

FIG. 10B is a graph of inhibition of RANTES secretion by the indicated agents. Agent #7 is human C15 peptide having the formula AGEDPHSFYFPGQFA (SEQ ID NO: 1).

FIG. 11 is graphs that exemplify that the depicted cytokine message and protein levels are increased upon LPS stimulation of human DCs matured from monocytes.

FIG. 12A exemplifies a Western-blot of lysates from cells transfected with and without siRNA to PP2A subunit C. An SDS-PAGE gel was transferred to a PVDF membrane and probed with antibodies that recognize PP2A subunit C and GAPDH.

FIG. 12B exemplifies graphs showing the effect of PP2A subunit C knockdown using siRNA on IL-23 and IL-12p70 expression levels.

FIG. 12C exemplifies graphs showing the effect of LPS and LPS with okadaic acid (OA) stimulation on IL-23, IL-12p70, and IL-12p40 expression levels.

FIG. 13A exemplifies a schematic of prochimerin processing and proteolytic cleavage events.

FIG. 13B exemplifies a schematic of prochimerin processing and proteolytic cleavage events.

FIG. 14A exemplifies a graph showing the number of NLS psoriasis and LS psoriasis cells that are positive for Chemerin and ChemR23.

FIG. 14B exemplifies a biopsy of a psoriatic lesion demonstrating Chemerin and ChemR23 expression is elevated in psoriatic plaques.

FIG. 15 exemplifies a graph and mice showing topically applied human C15 peptide having the formula AGEDPHSFYFPGQFA (SEQ ID NO: 1) reduces imiquimod-induced dermal inflammation in vivo in a dose dependent manner and has anti-inflammatory activity.

FIG. 16 exemplifies a schematic of NF-κB-p65 control by phosphatase PP2A, PR70, and an exemplary PR70 competitive inhibitor agent.

FIG. 17A exemplifies a graph showing that human C15 peptide having the formula AGEDPHSFYFPGQFA (SEQ ID NO: 1) modulates NF-κB activity more potently than steroid.

FIG. 17B exemplifies a graph showing human C15 peptide having the formula AGEDPHSFYFPGQFA (SEQ ID NO: 1) modulates NF-κB activity more potently than steroid.

FIG. 18A exemplifies a schematic of macrophage secretion of cytokines and inhibition thereof by an exemplary PR70 competitive inhibitor agent of the disclosure.

FIG. 18B exemplifies a graph showing that secretion of the shown NF-κB-regulated cytokines are reduced in mouse macrophages treated with human C15 peptide having the formula AGEDPHSFYFPGQFA (SEQ ID NO: 1).

FIG. 18C exemplifies a graph showing over a 90% reduction of newly synthesized RANTES in human macrophages treated with human C15 peptide having the formula AGEDPHSFYFPGQFA (SEQ ID NO: 1). The exemplary agent had a 10⁶ time greater potency than steroid (Dex).

FIG. 19A exemplifies anti IL-12p40 Ab staining of mature DC cells.

FIG. 19B exemplifies a graph of relative gene expression in non-plaque and plaque.

FIG. 19C exemplifies a graph showing plaque IL-23 levels in the indicated samples.

FIG. 20A exemplifies a schematic of PR70 binding to PP2A core enzyme.

FIG. 20B exemplifies a schematic of p65 phosphorylation at Ser 536.

FIG. 21 exemplifies a schematic of IL-23 secretion and regulation.

FIG. 22 exemplifies a schematic of a feedback loop that normally sustains inflammation without intervention.

FIG. 23A exemplifies a schematic of the effect an exemplary PR70 competitive inhibitor agent of the disclosure has on displacing or preventing PR70 binding to PP2A core enzyme.

FIG. 23B exemplifies a schematic of the effect an exemplary PR70 competitive inhibitor agent of the disclosure has on displacing or preventing PR70 binding to PP2A core enzyme and the effects on localization.

FIG. 24 exemplifies a schematic of the effect an exemplary PR70 competitive inhibitor agent of the disclosure has on stabilizing inactive NF-κB forms.

FIG. 25 exemplifies a schematic of inhibition of IL-23 secretion by an exemplary PR70 competitive inhibitor agent of the disclosure.

FIG. 26 exemplifies a schematic of TNFR and p75NTR signaling cascade.

FIG. 27 exemplifies the amino acid sequences of human PR70 (SEQ ID NO: 264) and human PR72 (SEQ ID NO: 265).

### DETAILED DESCRIPTION OF THE DISCLOSURE

Several aspects are described below with reference to example applications for illustration. It should be understood that numerous specific details, relationships, and methods are set forth to provide a full understanding of the features described herein. Those having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### I. Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 20, up to, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed. The term “about” has the meaning as commonly understood by one of ordinary skill in the art. In skill in the art. In some embodiments, the term “about” refers to ±10%. In some embodiments, the term “about” refers to ±5%.

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a human. In some embodiments, the mammal is a non-human. None of the terms require or are limited to situation characterized by the supervision (e.g. constant or intermittermittent) of a health care worker (e.g. a doctor, a registered nurse, a nurse practitioner, a physician's assistant, an orderly, or a hospice worker).

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or to an individual at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to an individual simultaneously in the form of a single entity or dosage. The term “non-fixed combination” means that at least one of the agents described herein, and at least one co-agent, are administered to an individual as separate entities either simultaneously, concurrently or sequentially with variable intervening time limits, wherein such administration provides effective levels of the two or more agents in the body of the individual. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single individual, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer stem cell. By way of non-limiting example, when the disease or condition is psoriasis, in certain instances, the result is a decrease in redness, scaliness and/or thickness of psoriatic plaques. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the agents and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “dex” as used herein, refers to the corticosteroid dexamathasone

### II. Agents for Use in Treating Human Diseases

Certain inflammatory conditions are stimulated, in part, by signals that activate the classical NF-κB pathway. Stimulation of NF-κB induces tissue damage and exacerbates inflammatory conditions.

The B″/PR72 family of protein phosphatase 2A (PP2A) is an important PP2A family involved in diverse cellular processes, and uniquely regulated by calcium binding to the regulatory subunit. The PR70 subunit in this family interacts with cell division control 6 (Cdc6), a cell cycle regulator important for control of DNA replication. Here, we report crystal structures of the isolated PR72 and the trimeric PR70 holoenzyme at a resolution of 2.1 and 2.4 Å, respectively, and in vitro characterization of Cdc6 dephosphorylation. The holoenzyme structure reveals that one of the PR70 calcium-binding motifs directly contacts the scaffold subunit, resulting in the most compact scaffold subunit conformation among all PP2A holoenzymes. PR70 also binds distinctively to the catalytic subunit near the active site, which is required for PR70 to enhance phosphatase activity toward Cdc6. Our studies provide a structural basis for unique regulation of B″/PR72 holoenzymes by calcium ions, and suggest the mechanisms for precise control of substrate specificity among PP2A holoenzymes.

Nuclear factor-κB (NF-κB) represents a group of five proteins, namely c-Rel, Rel A (p65), Rel B, NF-κB1 (p50 and p105), and NF-κB2 (p52). Ways to modulate NF-κB expression therapeutically have focused on its active-inactive state transition mechanisms. NF-κB is regulated by a family of inhibitors, called IB. In an inactive state, NF-κB is present in the cytoplasm as a heterotrimer consisting of p50, p65, and IκBα subunits. In response to an activation signal, the IκBα subunit is phosphorylated at serine residues 32 and 36, ubiquitinated at lysine residues 21 and 22, and degraded through the proteosomal pathway, thus exposing the nuclear localization signals on the p50-p65 heterodimer. The p65 is then phosphorylated, leading to the nuclear translocation and binding to a specific sequence in DNA, which in turn results in transcriptions of various genes including cytokines (e.g., Il-1β, IL-17, IL-23, TNFα, RANTES), cyclin D1, cyclooxygenase (COX)-2 and matrix metalloproteinase (MMP)-9.

NF-κB regulates the expression of a number of genes whose products are involved in inflammation, viral replication, carcinogenesis, antiapoptosis, invasion and metastasis. Specific adhesion molecules, chemokines, inflammatory cytokines, and cell cycle regulatory genes are affected. Thus agents that can suppress NF-κB activation have the potential to be treatments for diseases or disorders (e.g., inflammatory diseases and cancer).

The p65 subunit of NF-κB, which contains at least two strong transactivation domain (TAD) within the C terminus (TA1 30 amino acid; TA2 90 amino acid), when activated undergoes phosphorylation. The site of phosphorylation and kinase responsible for the phosphorylation has been controversial. For instance, phosphorylation can occur at Ser 276 by protein kinase A, at Ser 529 by casein kinase 11 (32-34), at Ser 536 by IKKβ, and at Ser 471 by PKCε. In addition, glycogen synthase kinase-3b and by Ca2+/calmodulin-dependent protein kinase IV can phosphorylate p65-TAD.

Some embodiments provided herein describe agents that are specific inhibitors of NF-κB activation. In some embodiments, the inhibitors of NF-κB activate PP2A. In some embodiments, the inhibitors of NF-κB stabilize PP2A. In some embodiments, the inhibitors of NF-κB stabilize a PP2A core enzyme. In some embodiments, the agent that is an inhibitor of NF-κB activates NF-κB/PP2A complex. In some embodiments, the agent that is an inhibitor of NF-κB stabilizes NF-κB/PP2A complex. In some embodiments, the activation of PP2A reverses kinase mediated activation of NF-κB. In some instances, the activations of PP2A reverses kinase mediated activation of NF-κB through a dephosphorylation of one or more sites present in the p65. Some embodiments provided herein describe a method of inhibiting NF-κB activation, the method comprising contacting a cell with a composition comprising any one of the active agents described herein. In some embodiments, the inhibitors of NF-κB control PP2A subcellular localization (i.e. nuclear vs cytosolic). In some embodiments, the inhibitors of NF-κB control PP2A subcellular localization (i.e. nuclear vs cytosolic) and increase PP2A core enzyme in the nucleus.

In some embodiments, the agent that is an inhibitor is linked with a protein transduction peptide or cell penetrating peptide. In some embodiments, the protein transduction peptide or cell penetrating peptide is a short peptide sequence that permits the inhibitor to cross the cell membrane. In some instances, the peptide-linked inhibitors enter cells without any receptors. In some embodiments, any one of the agents that are inhibitors described herein suppresses NF-κB activation induced by a variety of inflammatory stimuli. In other embodiments, any one of the agent inhibitors described herein suppresses NF-κB-mediated gene transcription apoptosis induced by TNF, INFγ, LPS, zymosan and other stimuli.

Provided herein in some embodiments is an agent that is an agent that is an inhibitor of the nuclear transcription factor NF-κB useful for the treatment of diseases associated with inflammation, viral replication, carcinogenesis, antiapoptosis, invasion, and metastasis. In some embodiments, the agent that is an inhibitor of NF-κB is not a peptide from the C-terminus of Chemerin. In some embodiments, the peptide that is not from the C-terminus of Chemerin is an agonist of PP2A. In some embodiments, the peptide that is not from the C-terminus of Chemerin is not C15. In some embodiments, the peptide from the C-terminus of Chemerin stabilizes the association of PP2A core enzyme with NF-κB. In some embodiments, the agents that are inhibitors described herein suppress INFγ/LPS induced NF-κB activation in vitro. In other embodiments, the agents that are inhibitors described herein suppress INFγ/LPS induced NF-κB activation in vivo. In certain embodiments, the agents described herein and other agonists or antagonists of PP2A are especially useful in the treatment of diseases which respond to steroid therapy (e.g., dexamethasone or other potent glucocortico steroids known to inhibit NF-κB activation).

Phosphorylation of p65 enhances its binding to the DNA. Some embodiments provided herein describe an agent that activates PP2A to dephosphorylate p65. In some embodiments, activation of PP2A to dephosphorylate p65 maintains NF-κB in a quiescent state outside the nucleus. In some embodiments, an active agent described herein prevents transcription of proteins related to inflammatory diseases, cancer, metastasis, viral infections and other diseases regulated by NF-dB.

In some instances, PP2A negatively regulates the expression and secretion of IL-23 in human dendritic cells. In some instances, PP2A negatively regulates the expression and secretion of IL-23 in human dendritic cells and reduces the phosphorylation of IKKβ (Ser 181) and NF-κB 536. In some instances, PP2A negatively regulates the expression and secretion of IL-23 in human dendritic cells and reduces the phosphorylation of IKKβ (Ser 181) and NF-κB Ser 536 but not Ser 276. In some embodiments, activation of PP2A controls the activity of NF-κB directly via dephosphorylation of Ser 536. In other embodiments, activation of PP2A controls the activity of NF-κB indirectly by regulation of IKKβ which controls the phosphorylation of IκB, a regulator of NF-κB. In some embodiments, a stimulator of PP2A described herein blocks the transcription and/or secretion of IL-23 in dendritic cells. In some embodiments, a stimulator of PP2A described herein is used to treat psoriatic plaques and/or control the disease.

The balance of protein kinase and phosphatase activities toward key proteins is central to many aspects of cellular physiology. Compared with kinases, protein phosphatases have received little attention, and appreciation that they may be just as precisely regulated as the enzymes whose action they oppose is relatively recent. Also the balance of subcellular localization of PP2A as regulated by PR70 has received little attention or appreciation.

PP2A is highly conserved in eukaryotes. It constitutes between 0.3% and 1% of total protein in mammalian cells and supplies the majority of soluble phosphatase activity toward phospho-serine and -threonine. PP2A is a holoenzyme of two or three subunits. A 36-kDa catalytic or C subunit complexes with a 65-kDa scaffolding A subunit to form the AC core enzyme; the core enzyme can bind a third, variable subunit to form the PP2A heterotrimer. In mammals, A and C subunits are each encoded by two highly similar genes, with A and C isoforms being more abundant. Regulatory, or B subunits, are encoded by three multigene families. Several PP2A regulatory subunits show restricted tissue and subcellular expression. Proteins encoded by DNA tumor viruses, SV40 small t and polyoma virus small and middle T antigen, are a fourth group of proteins that bind to the PP2A core enzyme and subvert its activity as a suppressor of cellular transformation. The AC dimer has also been shown to interact with other proteins, including the WD repeat containing proteins striatin and SG2NA.

The interaction of regulatory B subunits with the core dimer is critical for PP2A function and combines with the core AC subunits to form the ABC holoenzyme. The regulatory subunits can specifically target PP2A to substrates, signaling complexes, and subcellular localizations. There are four classes of regulatory B subunits termed R2, R3, R4, and R5, using nomenclature derived from their official human gene symbols. The diversity of regulatory subunits gives rise to multiple PP2A holoenzymes and accounts for the ability of PP2A to regulate diverse cellular processes. The presence of regulatory subunits affects the kinetics of dephosphorylation. In some instances, the regulatory subunits recruit substrates to PP2A. Genetic analysis in Saccharomyces cerevisiae, insect-cells, and mammalian cells also indicate that specific biological functions of PP2A are mediated by distinct regulatory subunits. In other instances, the regulatory subunits direct the AC core of PP2A away from substrates.

The R2 subunits are the best characterized family of B regulatory subunits of PP2A and consists of 4 members (α, β, γ, and δ). The R2α subunit targets PP2A to the Ras-Raf-Mek-Erk pathway, and regulates signaling at multiple steps of this pathway. Knockdown of the R2 subunit in Drosophila S2 cells by RNAi or displacement of R2α from the core dimer by the SV40 small tumor antigen (small-t) activates the mitogen activated protein kinase (MAPK), Erk. Once thought to primarily be a negative regulator of the Ras-Raf-Mek-Erk pathway, it is now known that PP2A also positively regulates the pathway. The R2α associates with Kinase Suppressor of Ras (KSR) and Raf-1 and promotes dephosphorylation of inhibitory phosphorylation sites on these substrates. The R2α and R2β subunits also target PP2A to microtubules via a heat labile anchoring activity present in microtubule-associated and microtubule-interacting proteins. The R2α and R2β subunits also target PP2A to the microtubule associated protein, tau. Over-expression of tau with SV40 small-t results in hyperphosphorylation of tau on multiple sites, dissociation of tau from microtubules, and destabilization of microtubules.

The R3 subunit family consists of 3 members (PR72, PR59, and PR70). The gene encoding PR72 produces two alternatively spliced transcripts encoding proteins of 72 and 130 kDa. PR72 can target PP2A to the Wnt signaling cascade by interacting with the human Naked cuticle protein. Over-expression of PR72 results in repression of the classical Wnt signaling cascade and the presence of PR72 is required for the inhibitory effect of Naked cuticle on Wnt signaling. The members of this family also have been implicated in targeting PP2A to proteins involved in cell cycle regulation. The splice variant PR130 targets PP2A to the scaffolding protein centrosome and Golgi localized PKN-associated protein (CG-NAP). CG-NAP anchors a signaling complex to the centrosome and golgi apparatus in a cell cycle dependent manner. PR59 was discovered in a yeast two hybrid screen with the retinoblastoma-related protein p107 as bait. Overexpression of PR59 results in dephosphorylation of p107 and cell cycle arrest in G1 phase. PR70 was identified in a yeast two hybrid screen with the DNA replication protein Cdc6 as bait. The interaction of PR70 with Cdc6 is discussed in more detail in the next section.

Regulatory subunits may impart specific functions to PP2A holoenzymes. For example, B-family regulatory subunits may regulate cytoskeletal protein assembly, B subunits may participate in the developmental Wnt/b-catenin signal transduction cascade, and B subunits may control the G1-S cell cycle transition. Adenovirus type 5 can induce apoptosis by interaction of its E4orf4 protein with the B subunit of PP2A. In vitro, regulatory subunits can affect enzymatic activity and substrate specificity of PP2A. Regulatory subunits can target PP2A holoenzymes to distinct subcellular compartments.

In, the crystal structure of the scaffolding A subunit of PP2A, the A subunit is a hook-shaped protein made up almost entirely of 15 imperfect repeats, each about 40 amino acids long. Each of these HEAT repeats (named after proteins that contain them: huntingtin, elongation factor, A subunit, and TOR kinase) consists of two antiparallel, amphipathic a-helices.

The B subunit R2, R3 and R5 families contain a highly conserved FXF/Y sequence motif. In the Human PR70 R3 family member, this is a PTFYFP (SEQ ID NO: 8) sequence. Mutation of any one of the FYF (SEQ ID NO: 2) residues to alanine decreases the binding of PR70 to the PP2A core enzyme. Overexpression of the PR70 subunit inhibits NF-κB activation and transcription of reporter genes.

In some embodiments, the agent that is not the anti-inflammatory peptide C15 (AGEDPHSFYFPGQFA (SEQ ID NO: 1)) derived from the C-terminus of Chemerin by proteolysis, is a peptide that contains an FYF (SEQ ID NO: 2) sequence and is broadly anti-inflammatory against TNFα, INFγ, LPS and zymosan induced inflammation. In some embodiments, the agent that is not C15 exerts its broad anti-inflammatory activity by acting as a decoy of the B subunit of PP2A.

In some instances, an active agent described herein (e.g., an agent that is not C15) binds to a PP2A core enzyme. In some embodiments, an active agent described herein (e.g., a C15 analog) binds to PP2A core enzyme as a dominant positive B subunit surrogate and stabilizes the PP2A core enzyme relative to the holoenzyme. In some embodiments, following the binding of an active agent described herein (e.g., an agent that is not C15) to a PP2A core enzyme, the PP2A core enzyme associates with NF-κB, IKKβ, β-arrestin, Akt, and/or other proteins in the Classical NF-κB pathway and holds them in an inactive, non-phosphorylated state. In some embodiments, an active agent described herein (e.g., a C15 analog) modulates NF-κB. In certain embodiments, the active agent inhibits or suppresses NF-κB.

In other embodiments, an active agent described herein (e.g., an agent that is not C15) modulates IKK (e.g., IKKβ, IKKα, NEMO). In certain embodiments, the active agent inhibits or suppresses IKK. In certain embodiments, the active agent inhibits or suppresses the phosphorylation state of IKK substrates. In certain embodiments, the active agent inhibits or suppresses IKKβ activity. In some embodiments, an active agent described herein (e.g., a C15 analog) modulates β-arrestin. In certain embodiments, the active agent inhibits or suppresses β-arrestin. In some embodiments, an active agent described herein (e.g., an agent that is not C15) modulates Akt. In certain embodiments, the active agent inhibits or suppresses Akt. In some instances, PP2A negatively regulates the expression and secretion of IL-23 by human dendritic cells. In some instances, PP2A negatively regulates the expression and secretion of IL-23 by myeloid dendritic cells in a psoriatic plaque.

In some embodiments, an active agent described herein (e.g., a C15 analog) suppresses a cytokine associated with a Th-1 response in a patient. In some embodiments, the cytokine is IL-12, IL-17, IFNγ, TNFα, or IL-23.

In some embodiments, an active agent described herein (e.g., an agent that is not C15) modulates the PP2A/IKKα/IκBα/p65 NF-κB pathway. In some embodiments, an active agent described herein (e.g., a C15 analog) inhibits or suppresses the PP2A/IKKα/IκBα/p65 NF-κB pathway. In some embodiments, the active agent (e.g., an agent that is not C15) modulates the expression of pro-apoptotic genes, TNFα, TRAILR1, and/or TRAILR2. In some embodiments, the active agent inhibits or suppresses the expression of pro-apoptotic genes, TNFα, TRAILR1, and/or TRAILR2. In some embodiments, the active agent (e.g., a C15 analog) modulates the Akt/IKKβ/NF-κB pathway. In some embodiments, the active agent (e.g., an agent that is not C15) inhibits or suppresses the Akt/IKKβ/NF-κB pathway. In some embodiments, an active agent described herein (e.g., a C15 analog) modulates or suppresses the Ras-Raf-Mek-Erk pathway.

In some embodiments, an active agent described herein (e.g., an agent that is not C15) modulates the mammalian target of rapamycin (mTOR) pathway. In certain embodiments, an active agent described herein (e.g., a C15 analog) inhibits or suppresses the mammalian target of rapamycin (mTOR) pathway. In some embodiments, the active agent modulates TNF receptor associated factors (TRAF). In some embodiments, the active agent inhibits or suppresses a TRAF. In other embodiments, the active agent modulates Toll-like receptors (e.g., TLR3, TLR4, etc.). In certain embodiments, the active agent inhibits or suppresses Toll-like receptors (e.g., TLR3, TLR4, etc.). In some embodiments, the active agent modulates interleukin 1, TRADD, MyD88, IRAK, RIP (receptor interacting proteins), PI3K, MEKK, RANKL, IGF, or PtdIns(3,4,5)P3. In certain embodiments, the active agent inhibits or suppresses interleukin 1, TRADD, MyD88, IRAK, RIP (receptor interacting proteins), PI3K, MEKK, RANKL, IGF, or PtdIns(3,4,5)P3. In some embodiments, the active agent modulates or suppresses Bcl-XL, Bcl-2, vascular endothelial growth factor (VEGF), or interleukin-8.

In some embodiments, an agent has an amino acid sequence with at least 80%, 85%, 90%, 95%, or 100% sequence identity to LSINIPRFYFPEGLP (SEQ ID NO: 32), LSINIPRXFYFPEGLP (SEQ ID NO: 33), or LSINIPXRFYFPEGLP (SEQ ID NO: 34); wherein the agent is not a human C15 peptide having the formula AGEDPHSFYFPGQFA (SEQ ID NO: 1). In some embodiments, the agent has an amino acid sequence with at least 80%, 85%, 90%, 95%, or 100% sequence identity to TSQSIPTFYFPRGRP (SEQ ID NO: 35), TSQSIPXTFYFPRGRP (SEQ ID NO: 36), or TSQSIPTXFYFPRGRP (SEQ ID NO: 37); wherein the agent is not a human C15 peptide having the formula AGEDPHSFYFPGQFA (SEQ ID NO: 1). In some embodiments, the agent has an amino acid sequence with at least 80%, 85%, 90%, 95%, or 100% sequence identity to human PR70 or a portion thereof; wherein the agent is not a human C15 peptide having the formula AGEDPHSFYFPGQFA (SEQ ID NO: 1). In some embodiments, the agent is selected from the group consisting of: AGEDPHGYFLPGQFA (SEQ ID NO: 38), AGEDPHSFYFPGQFA (SEQ ID NO: 1), AGEDPHSFYFPGQFAF, AGEDPHSFYFPGQFAFS, PHSFYFPGQFA (SEQ ID NO: 41), XGEDPHSFYFPGQFA (SEQ ID NO: 42), AXEDPHSFYFPGQFA (SEQ ID NO: 43), AGXDPHSFYFPGQFA (SEQ ID NO: 44), AGEXPHSFYFPGQFA (SEQ ID NO: 45), AGEDXHSFYFPGQFA (SEQ ID NO: 46), AGEDPXSFYFPGQFA (SEQ ID NO: 47), AGEDPHXFYFPGQFA (SEQ ID NO: 48), AGEDPHSXYFPGQFA (SEQ ID NO: 49), AGEDPHSFXFPGQFA (SEQ ID NO: 50), AGEDPHSFYXPGQFA (SEQ ID NO: 51), AGEDPHSFYFXGQFA (SEQ ID NO: 52), AGEDPHSFYFPXQFA (SEQ ID NO: 53), AGEDPHSFYFPGXFA (SEQ ID NO: 54), AGEDPHSFYFPGQXA (SEQ ID NO: 55), AGEDPHSFYFPGQFX (SEQ ID NO: 56), AGEDPHSXYX′PGQFA (SEQ ID NO: 57), AGEDPHSXX′X″PGQFA (SEQ ID NO: 58), UGEDPHSFYFPGQFA (SEQ ID NO: 59), AUEDPHSFYFPGQFA (SEQ ID NO: 60), AGUDPHSFYFPGQFA (SEQ ID NO: 61), AGEUPHSFYFPGQFA (SEQ ID NO: 62), AGEDUHSFYFPGQFA (SEQ ID NO: 63), AGEDPUSFYFPGQFA (SEQ ID NO: 64), AGEDPHUFYFPGQFA (SEQ ID NO: 65), AGEDPHSUYFPGQFA (SEQ ID NO: 66), AGEDPHSFUFPGQFA (SEQ ID NO: 67), AGEDPHSFYUPGQFA (SEQ ID NO: 68), AGEDPHSFYFUGQFA (SEQ ID NO: 69), AGEDPHSFYFPUQFA (SEQ ID NO: 70), AGEDPHSFYFPGUFA (SEQ ID NO: 71), AGEDPHSFYFPGQUA (SEQ ID NO: 72), AGEDPHSFYFPGQFU (SEQ ID NO: 73), AGEDPHSUYU′PGQFA (SEQ ID NO: 74), AGEDPHSUU′U″PGQFA (SEQ ID NO: 75), and any combination thereof, wherein X is an unnatural amino acid or a natural amino acid or absent, wherein X, X′ and X″ are the same or different, wherein U is an unnatural amino acid, and wherein U, U′ and U″ are the same or different. In some embodiments, a U is homo-serine. In some embodiments, a U is p-chlorophenylalanine.

In some embodiments, the agent does not consist of any one of the peptides selected from the group consisting of PHGYFLPGQPA (SEQ ID NO: 76); PHGYFLPGQFAF; PHGYFLPGQFAFS; AGEDPHGYFLPGQFA (SEQ ID NO: 38); AGEDPHGYFLPGQFAF (SEQ ID NO: 39); AGEDPHGYFLPGQFAFS (SEQ ID NO: 40); DPHGYFLPGQFA (SEQ ID NO: 81); EDPHGYFLPGQFA (SEQ ID NO: 82); GEDPHGYPLPGQFA (SEQ ID NO: 83); DPHGYFLPGQFAF (SEQ ID NO: 84); EDPHGYFLPGQFAF (SEQ ID NO: 85); GEDPHGYFLPGQFAF (SEQ ID NO: 86); DPHGYFLPGQFAFS (SEQ ID NO: 87); EDPHGYFLPGQFAFS (SEQ ID NO: 88); GEDPHGYFLPGQFAFS (SEQ ID NO: 89); PHSEYFPGQFA (SEQ ID NO: 90); PHSFYFPGQFAF (SEQ ID NO: 91); PHSFYFPGQFAFS (SEQ ID NO: 92); AGEDPHSFYFPGQFA (SEQ ID NO: 1); AGEDPHSFYPPGQFAF (SEQ ID NO: 93); AGEDPHSFYFPGQFAFS; DPHSFYFPGQFA (SEQ ID NO: 94); EDPHSFYFPGQFA (SEQ ID NO: 95); GEDPHSPYFPGQFA (SEQ ID NO: 96); DPHSFYPPGQFAF (SEQ ID NO: 97); EDPHSFYFPGQFAF (SEQ ID NO: 98); GEDPHSFYFPGQFAF (SEQ ID NO: 99); DPHSFYFPGQFAPS (SEQ ID NO: 100); EDPHSFYFPGQFAFS (SEQ ID NO: 101); GEDPHSFYFPGQFAFS (SEQ ID NO: 102); AQAGEDPHGYFLPGQFAFS (SEQ ID NO: 103); and QRAGEDPHSFYFPGQFAFS (SEQ ID NO: 104). In some embodiments, the agent does not comprise an amino acid sequence selected from the group consisting of PHGYFLPGQPA (SEQ ID NO: 76); PHGYFLPGQFAF; PHGYFLPGQFAFS; AGEDPHGYFLPGQFA (SEQ ID NO: 38); AGEDPHGYFLPGQFAF (SEQ ID NO: 39); AGEDPHGYFLPGQFAFS (SEQ ID NO: 40); DPHGYFLPGQFA (SEQ ID NO: 81); EDPHGYFLPGQFA (SEQ ID NO: 82); GEDPHGYPLPGQFA (SEQ ID NO: 83); DPHGYFLPGQFAF (SEQ ID NO: 84); EDPHGYFLPGQFAF (SEQ ID NO: 85); GEDPHGYFLPGQFAF (SEQ ID NO: 86); DPHGYFLPGQFAFS (SEQ ID NO: 87); EDPHGYFLPGQFAFS (SEQ ID NO: 88); GEDPHGYFLPGQFAFS (SEQ ID NO: 89); PHSEYFPGQFA (SEQ ID NO: 90); PHSFYFPGQFAF (SEQ ID NO: 91); PHSFYFPGQFAFS (SEQ ID NO: 92); AGEDPHSFYFPGQFA (SEQ ID NO: 1); AGEDPHSFYPPGQFAF (SEQ ID NO: 93); AGEDPHSFYFPGQFAFS; DPHSFYFPGQFA (SEQ ID NO: 94); EDPHSFYFPGQFA (SEQ ID NO: 95); GEDPHSPYFPGQFA (SEQ ID NO: 96); DPHSFYPPGQFAF (SEQ ID NO: 97); EDPHSFYFPGQFAF (SEQ ID NO: 98); GEDPHSFYFPGQFAF (SEQ ID NO: 99); DPHSFYFPGQFAPS (SEQ ID NO: 100); EDPHSFYFPGQFAFS (SEQ ID NO: 101); GEDPHSFYFPGQFAFS (SEQ ID NO: 102); AQAGEDPHGYFLPGQFAFS (SEQ ID NO: 103); and QRAGEDPHSFYFPGQFAFS (SEQ ID NO: 104). In some embodiments, the agent has less than about 30%, 40%, 50%, 60%, 70%, 80%, 90% 95%, or 99% sequence identity with one or more of the peptides selected from the group consisting of PHGYFLPGQPA (SEQ ID NO: 76); PHGYFLPGQFAF (SEQ ID NO: 77); PHGYFLPGQFAFS; AGEDPHGYFLPGQFA (SEQ ID NO: 38); AGEDPHGYFLPGQFAF (SEQ ID NO: 39); AGEDPHGYFLPGQFAFS (SEQ ID NO: 40); DPHGYFLPGQFA (SEQ ID NO: 81); EDPHGYFLPGQFA (SEQ ID NO: 82); GEDPHGYPLPGQFA (SEQ ID NO: 83); DPHGYFLPGQFAF (SEQ ID NO: 84); EDPHGYFLPGQFAF (SEQ ID NO: 85); GEDPHGYFLPGQFAF (SEQ ID NO: 86); DPHGYFLPGQFAFS (SEQ ID NO: 87); EDPHGYFLPGQFAFS (SEQ ID NO: 88); GEDPHGYFLPGQFAFS (SEQ ID NO: 89); PHSEYFPGQFA (SEQ ID NO: 90); PHSFYFPGQFAF (SEQ ID NO: 91); PHSFYFPGQFAFS (SEQ ID NO: 92); AGEDPHSFYFPGQFA (SEQ ID NO: 1); AGEDPHSFYPPGQFAF (SEQ ID NO: 93); AGEDPHSFYFPGQFAFS; DPHSFYFPGQFA (SEQ ID NO: 94); EDPHSFYFPGQFA (SEQ ID NO: 95); GEDPHSPYFPGQFA (SEQ ID NO: 96); DPHSFYPPGQFAF (SEQ ID NO: 97); EDPHSFYFPGQFAF (SEQ ID NO: 98); GEDPHSFYFPGQFAF (SEQ ID NO: 99); DPHSFYFPGQFAPS (SEQ ID NO: 100); EDPHSFYFPGQFAFS (SEQ ID NO: 101); GEDPHSFYFPGQFAFS (SEQ ID NO: 102); AQAGEDPHGYFLPGQFAFS (SEQ ID NO: 103); and QRAGEDPHSFYFPGQFAFS (SEQ ID NO: 104). In some embodiments, the agent has less than about 80%, 85%, 90%, 91%, 92%, 93%, 94%, 95%, 96%, 97%, 98%, 99%, 99.5%, 99.6%, 99.7%, 99.8%, or 99.9% sequence identity to a naturally occurring chemerin C15 peptide. In some embodiments, the agent has less than about 80%, 85%, 90%, 91%, 92%, 93%, 94%, 95%, 96%, 97%, 98%, 99%, 99.5%, 99.6%, 99.7%, 99.8%, or 99.9% sequence identity to a human C15 peptide having the formula AGEDPHSFYFPGQFA (SEQ ID NO: 1).

In some embodiments, the agent is a chimeric sequence comprising a human chemerin sequence and a sequence from a non-human organism. In some embodiments, the agent binds to PR70. In some embodiments, the agent binds to PR70 with an affinity of less than 10 nM, 1 nM, 100 pM, 10 pM, or 1 pM or less. In some embodiments, the agent binds to an amino acid of an FYF (SEQ ID NO: 2) sequence of PR70. In some embodiments, the agent binds to amino acid Phe 128 of PR70. In some embodiments, the agent binds to a same region of PP2A as PR70. In some embodiments, the same region of PP2A comprises a PP2A region that binds to an amino acid of an FYF (SEQ ID NO: 2) sequence of PR70.

In some embodiments, the agent is a small molecule. In some embodiments, the agent is an antibody. In some embodiments, the agent is a nucleic acid. In some embodiments, the nucleic acid is RNA. In some embodiments, the nucleic acid is DNA. In some embodiments, the agent is not a peptide.

In some embodiments, the agent is a peptide. In some embodiments, the peptide comprises the amino acid sequence FYF (SEQ ID NO: 2). In some embodiments, the peptide comprises the amino acid sequence FYFP (SEQ ID NO: 3). In some embodiments, the peptide comprises the amino acid sequence PFYFP (SEQ ID NO: 4). In some embodiments, the peptide comprises the amino acid sequence PXFYFP (SEQ ID NO: 5), wherein X is any amino acid or analog thereof. In some embodiments, the peptide comprises the amino acid sequence PSFYFP (SEQ ID NO: 7), wherein X is any amino acid or analog thereof. In some embodiments, the peptide comprises the amino acid sequence PTFYFP (SEQ ID NO: 8), wherein X is any amino acid or analog thereof. In some embodiments, the peptide comprises the amino acid sequence XFYF (SEQ ID NO: 110), FYFX (SEQ ID NO: 111), FYFP (SEQ ID NO: 3), XFYFX (SEQ ID NO: 112), XFYFP (SEQ ID NO: 113), XXFYF (SEQ ID NO: 114), PXFYF (SEQ ID NO: 115), FYFXX (SEQ ID NO: 116), FYFPX (SEQ ID NO: 117), XXFYFX (SEQ ID NO: 118), PXFYFX (SEQ ID NO: 119), XXFYFP (SEQ ID NO: 120), PXFYFP (SEQ ID NO: 5), XXXFYF (SEQ ID NO: 121), XPXFYF (SEQ ID NO: 122), PXXFYF (SEQ ID NO: 123), XXXFYFX (SEQ ID NO: 124), XXXFYFP (SEQ ID NO: 125), PXXFYFX (SEQ ID NO: 126), XPXFYFX (SEQ ID NO: 127), PXXFYFP (SEQ ID NO: 9), XPXFYFP (SEQ ID NO: 128), XXXFYFXX (SEQ ID NO: 129), PXXFYFXX (SEQ ID NO: 130), XPXFYFXX (SEQ ID NO: 131), XXXFYFPX (SEQ ID NO: 132), PXXFYFPX (SEQ ID NO: 133), or XPXFYFPX (SEQ ID NO: 134), wherein X is any amino acid or analog thereof. In some embodiments, the peptide comprises the amino acid sequence (S/T)FYF (SEQ ID NO: 135), FYFX (SEQ ID NO: 111), FYFP (SEQ ID NO: 3), (S/T)FYFX (SEQ ID NO: 136), (S/T)FYFP (SEQ ID NO: 137), X(S/T)FYF (SEQ ID NO: 138), P(S/T)FYF (SEQ ID NO: 139), FYFXX, FYFPX, X(S/T)FYFX (SEQ ID NO: 140), P(S/T)FYFX (SEQ ID NO: 141), X(S/T)FYFP (SEQ ID NO: 142), P(S/T)FYFP (SEQ ID NO: 6), XX(S/T)FYF (SEQ ID NO: 143), XP(S/T)FYF (SEQ ID NO: 144), PX(S/T)FYF (SEQ ID NO: 145), XX(S/T)FYFX (SEQ ID NO: 146), XX(S/T)FYFP (SEQ ID NO: 147), PX(S/T)FYFX (SEQ ID NO: 148), XP(S/T)FYFX (SEQ ID NO: 149), PX(S/T)FYFP (SEQ ID NO: 9), XP(S/T)FYFP (SEQ ID NO: 150), XX(S/T)FYFXX (SEQ ID NO: 151), PX(S/T)FYFXX (SEQ ID NO: 152), XP(S/T)FYFXX (SEQ ID NO: 153), XX(S/T)FYFPX (SEQ ID NO: 154), PX(S/T)FYFPX (SEQ ID NO: 155), or XP(S/T)FYFPX (SEQ ID NO: 156), wherein X is any amino acid or analog thereof. In some embodiments, the peptide comprises the amino acid sequence H(S/T)FYF (SEQ ID NO: 157), H(S/T)FYFX (SEQ ID NO: 158), H(S/T)FYFP (SEQ ID NO: 159), XH(S/T)FYF (SEQ ID NO: 160), PH(S/T)FYF (SEQ ID NO: 161), XH(S/T)FYFX (SEQ ID NO: 162), XH(S/T)FYFP (SEQ ID NO: 163), PH(S/T)FYFX (SEQ ID NO: 164), PH(S/T)FYFP (SEQ ID NO: 165), XH(S/T)FYFXX (SEQ ID NO: 166), PH(S/T)FYFXX (SEQ ID NO: 167), XH(S/T)FYFPX (SEQ ID NO: 168), or PH(S/T)FYFPX (SEQ ID NO: 169), wherein X is any amino acid or analog thereof. In some embodiments, the peptide comprises a nuclear translocation signal sequence.

In some embodiments, the peptide comprises the amino acid sequence FYY (SEQ ID NO: 170). In some embodiments, the peptide comprises the amino acid sequence FYYP (SEQ ID NO: 171). In some embodiments, the peptide comprises the amino acid sequence PFYYP (SEQ ID NO: 172). In some embodiments, the peptide comprises the amino acid sequence PXFYYP (SEQ ID NO: 173), wherein X is any amino acid or analog thereof. In some embodiments, the peptide comprises the amino acid sequence PSFYYP (SEQ ID NO: 174), wherein X is any amino acid or analog thereof. In some embodiments, the peptide comprises the amino acid sequence PTFYYP (SEQ ID NO: 175), wherein X is any amino acid or analog thereof.

Peptide agents are also disclosed herein. Therapeutic peptide agents can modulate, e.g., the activity of NF-κB, such as by competitively inhibiting binding of a B subunit of PP2A (e.g., PR70) to a PP2A core enzyme. In some embodiments a peptide agent comprises a structure that mimics the 3-dimensional structure of a XFYF (SEQ ID NO: 110), FYFX (SEQ ID NO: 111), FYFP (SEQ ID NO: 3), XFYFX (SEQ ID NO: 112), XFYFP (SEQ ID NO: 113), XXFYF (SEQ ID NO: 114), PXFYF (SEQ ID NO: 115), FYFXX (SEQ ID NO: 116), FYFPX (SEQ ID NO: 117), XXFYFX (SEQ ID NO: 118), PXFYFX (SEQ ID NO: 119), XXFYFP (SEQ ID NO: 120), PXFYFP (SEQ ID NO: 5), XXXFYF (SEQ ID NO: 121), XPXFYF (SEQ ID NO: 122), PXXFYF (SEQ ID NO: 123), XXXFYFX (SEQ ID NO: 124), XXXFYFP (SEQ ID NO: 125), PXXFYFX (SEQ ID NO: 126), XPXFYFX (SEQ ID NO: 127), PXXFYFP (SEQ ID NO: 9), XPXFYFP (SEQ ID NO: 128), XXXFYFXX (SEQ ID NO: 129), PXXFYFXX (SEQ ID NO: 130), XPXFYFXX (SEQ ID NO: 131), XXXFYFPX (SEQ ID NO: 132), PXXFYFPX (SEQ ID NO: 133), or XPXFYFPX (SEQ ID NO: 134) sequence of a PP2A B subunit (e.g., a FYFP (SEQ ID NO: 3) sequence of PR70 and PR72) when bound to a PP2A core enzyme (i.e., PP2A holoenzyme). In some embodiments a peptide agent comprises a structure that mimics the 3-dimensional structure of a (S/T)FYF (SEQ ID NO: 135), FYFX (SEQ ID NO: 111), FYFP (SEQ ID NO: 3), (S/T)FYFX (SEQ ID NO: 136), (S/T)FYFP (SEQ ID NO: 137), X(S/T)FYF (SEQ ID NO: 138), P(S/T)FYF (SEQ ID NO: 139), FYFXX, FYFPX, X(S/T)FYFX (SEQ ID NO: 140), P(S/T)FYFX (SEQ ID NO: 141), X(S/T)FYFP (SEQ ID NO: 142), P(S/T)FYFP (SEQ ID NO: 6), XX(S/T)FYF (SEQ ID NO: 143), XP(S/T)FYF (SEQ ID NO: 144), PX(S/T)FYF (SEQ ID NO: 145), XX(S/T)FYFX (SEQ ID NO: 146), XX(S/T)FYFP (SEQ ID NO: 147), PX(S/T)FYFX (SEQ ID NO: 148), XP(S/T)FYFX (SEQ ID NO: 149), PX(S/T)FYFP (SEQ ID NO: 9), XP(S/T)FYFP (SEQ ID NO: 150), XX(S/T)FYFXX (SEQ ID NO: 151), PX(S/T)FYFXX (SEQ ID NO: 152), XP(S/T)FYFXX (SEQ ID NO: 153), XX(S/T)FYFPX (SEQ ID NO: 154), PX(S/T)FYFPX (SEQ ID NO: 155), or XP(S/T)FYFPX (SEQ ID NO: 156) sequence of a PP2A B subunit (e.g., a sequence of PR70 and PR72) when bound to a PP2A core enzyme (i.e., PP2A holoenzyme). In some embodiments a peptide agent comprises a structure that mimics the 3-dimensional structure of a H(S/T)FYF (SEQ ID NO: 157), H(S/T)FYFX (SEQ ID NO: 158), H(S/T)FYFP (SEQ ID NO: 159), XH(S/T)FYF (SEQ ID NO: 160), PH(S/T)FYF (SEQ ID NO: 161), XH(S/T)FYFX (SEQ ID NO: 162), XH(S/T)FYFP (SEQ ID NO: 163), PH(S/T)FYFX (SEQ ID NO: 164), PH(S/T)FYFP (SEQ ID NO: 165), XH(S/T)FYFXX (SEQ ID NO: 166), PH(S/T)FYFXX (SEQ ID NO: 167), XH(S/T)FYFPX (SEQ ID NO: 168), or PH(S/T)FYFPX (SEQ ID NO: 169) sequence of a PP2A B subunit (e.g., a sequence of PR70 and PR72) when bound to a PP2A core enzyme (i.e., PP2A holoenzyme).

In some embodiments a peptide agent comprises a structure that mimics the 3-dimensional structure of a FYF (SEQ ID NO: 2) sequence of a PP2A B subunit (e.g., a FYF (SEQ ID NO: 2) sequence of PR70 and PR72) when bound to a PP2A core enzyme (i.e., PP2A holoenzyme). In some embodiments a peptide agent comprises a structure that mimics the 3-dimensional structure of a FYFP sequence of a PP2A B subunit (e.g., a FYFP (SEQ ID NO: 3) sequence of PR70 and PR72) when bound to a PP2A core enzyme (i.e., PP2A holoenzyme). In some embodiments a peptide agent comprises a structure that mimics the 3-dimensional structure of a TFYFP (SEQ ID NO: 176) sequence of a PP2A B subunit (e.g., a TFYFP (SEQ ID NO: 176) sequence of PR70 and PR72) when bound to a PP2A core enzyme (i.e., PP2A holoenzyme). In some embodiments a peptide agent comprises a structure that mimics the 3-dimensional structure of a SFYFP (SEQ ID NO: 177) sequence of a PP2A B subunit (e.g., a SFYFP (SEQ ID NO: 177) sequence of PR70 and PR72) when bound to a PP2A core enzyme (i.e., PP2A holoenzyme). In some embodiments a peptide agent comprises a structure that mimics the 3-dimensional structure of a PSFYFP (SEQ ID NO: 7) sequence of a PP2A B subunit (e.g., a PSFYFP (SEQ ID NO: 7) sequence of PR70 and PR72) when bound to a PP2A core enzyme (i.e., PP2A holoenzyme). In some embodiments a peptide agent comprises a structure that mimics the 3-dimensional structure of a PTFYFP (SEQ ID NO: 8) sequence of a PP2A B subunit (e.g., a PTFYFP (SEQ ID NO: 8) sequence of PR70 and PR72) when bound to a PP2A core enzyme (i.e., PP2A holoenzyme).

In some embodiments, the peptide comprises a nuclear translocation signal sequence. In some embodiments, the nuclear translocation signal sequence comprises one or more gapped dipeptides linked to nucelar localization. For example, in some embodiments, the nuclear translocation signal sequence comprises 1, 2, 3, 4, 5, 6, or 7 or more gapped dipeptides. In some embodiments, the nuclear translocation signal sequence comprises one or more nested and gapped dipeptides linked to nucelar localization. In some embodiments, a gap bewteen a gapped dipeptide is 0, 1, 2, 3, 4, or 5 amino acids in length. In some embodiments, a gapped dipeptide is GP (SEQ ID NO: 12), DS (SEQ ID NO: 13), PS (SEQ ID NO: 14), PP (SEQ ID NO: 15), or PG (SEQ ID NO: 16). For example, in some embodiments, the nuclear translocation signal sequence comprises a gapped dipeptide GP (SEQ ID NO: 12) with a gap bewteen the gapped dipeptide of 2 amino acids (i.e. GXXP (SEQ ID NO: 17)). For example, in some embodiments, the nuclear translocation signal sequence comprises a gapped dipeptide DS with a gap bewteen the gapped dipeptide of 2 amino acids (i.e. DXXS (SEQ ID NO: 18)). For example, in some embodiments, the nuclear translocation signal sequence comprises a gapped dipeptide PS with a gap bewteen the gapped dipeptide of lamino acid (i.e. PXS (SEQ ID NO: 19)). For example, in some embodiments, the nuclear translocation signal sequence comprises a gapped dipeptide PP with a gap bewteen the gapped dipeptide of 5 amino acids (i.e. PXXXXXP (SEQ ID NO: 20)). For example, in some embodiments, the nuclear translocation signal sequence comprises a gapped dipeptide PG with a gap bewteen the gapped dipeptide of 0 amino acids (i.e. PG (SEQ ID NO: 16)). In some embodiments, any one gapped dipeptide may be sufficient for nuclear localization. In some embodiments, 2, 3, 4, 5, 6, 7 or more gapped dipeptides are sufficient for nuclear localization.

In some embodiments, the nuclear translocation signal sequence comprises the sequence: GXXPXS (SEQ ID NO: 21). In some embodiments, the nuclear translocation signal sequence comprises the sequence: GXDPXS (SEQ ID NO: 22). In some embodiments, the nuclear translocation signal sequence comprises the sequence: GXXPXXXXXPG (SEQ ID NO: 23). In some embodiments, the nuclear translocation signal sequence comprises the sequence: GXXPXXXXXP (SEQ ID NO: 24). In some embodiments, the nuclear translocation signal sequence comprises the sequence: DPXS (SEQ ID NO: 25). In some embodiments, the nuclear translocation signal sequence comprises the sequence: DPXSXXXP (SEQ ID NO: 26). In some embodiments, the nuclear translocation signal sequence comprises the sequence: DXXSXXXPG (SEQ ID NO: 27). In some embodiments, the nuclear translocation signal sequence comprises the sequence: PXSXXXP (SEQ ID NO: 28). In some embodiments, the nuclear translocation signal sequence comprises the sequence: PXSXXXPG (SEQ ID NO: 29). In some embodiments, the nuclear translocation signal sequence comprises the sequence: GxDPxSXXXPG (SEQ ID NO: 30). In some embodiments, the nuclear translocation signal sequence comprises the sequence: XGXDPXSXXXPGXXX (SEQ ID NO: 31).

In some embodiments, the agent consists of 6, 7, 8, 9, 10, 11, 12, 13, 14, 15, 16, 17, 18, 19, 20, 21, 22, 23, 24, 25, 26, 27, 28, 29, or 30 amino acids. In some embodiments, the agent suppresses NF-κB p65 to a level similar to the suppression by a corticosteroid, wherein the agent is at least about 2, 3, 4, 5, 6, 7, 8, 9, 10, 20, 40, 50, 60, 80, 90, 100, 150, 200, 300, 400, or 500, or more times potent than the corticosteroid.

In some embodiments, the agent further comprises a detectable label.

The therapeutic agents described herein can comprise one or more of, for example, small non-protein and non-nucleic acids, proteins, peptides, protein fragments, nucleic acids (DNA, RNAJ, PNA (peptide nucleic acids), or their derivatives or mimetics which can modulate, e.g., the activity of NF-κB, such as by competitively inhibiting binding of a B subunit of PP2A (e.g., PR70) to a PP2A core enzyme. In some embodiments, the therapeutic agents described herein comprise one or more small non-proteins. In some embodiments, the therapeutic agents described herein comprise one or more non-nucleic acids. In some embodiments, the therapeutic agents described herein comprise one or more proteins. In some embodiments, the therapeutic agents described herein comprise one or more peptides.

In some embodiments, the therapeutic agents described herein comprise one or more protein fragments.

In some embodiments, the therapeutic agents described herein comprise one or more nucleic acids (DNA, RNAJ, PNA (peptide nucleic acids), or their derivatives or mimetics.

Also disclosed herein are methods for determining affinities of one or more agents for a B subunit of PP2A (e.g., PR70) or to a PP2A core enzyme. In some embodiments, an agent described herein has a binding affinity of at least 10⁻⁷M (K_{D}), such as at least 10⁻⁸M, 10⁻⁹M, 10⁻¹⁰M, 10⁻¹¹M, 10⁻¹²M, 10⁻¹³M, 10⁻¹⁴M, 10⁻¹⁵M, or 10⁻¹⁶M, for a B subunit of PP2A (e.g., PR70) or to a PP2A core enzyme.

### Small Molecule Agents

Small molecule agents comprise one or more small molecules. Therapeutic small molecule agents can modulate, e.g., the activity of NF-κB, such as by competitively inhibiting binding of a B subunit of PP2A (e.g., PR70) to a PP2A core enzyme. In some embodiments a small molecule agent comprises a structure that mimics the 3-dimensional structure of a XFYF (SEQ ID NO: 110), FYFX (SEQ ID NO: 111), FYFP (SEQ ID NO: 3), XFYFX (SEQ ID NO: 112), XFYFP (SEQ ID NO: 113), XXFYF (SEQ ID NO: 114), PXFYF (SEQ ID NO: 115), FYFXX (SEQ ID NO: 116), FYFPX (SEQ ID NO: 117), XXFYFX (SEQ ID NO: 118), PXFYFX (SEQ ID NO: 119), XXFYFP (SEQ ID NO: 120), PXFYFP (SEQ ID NO: 5), XXXFYF (SEQ ID NO: 121), XPXFYF (SEQ ID NO: 122), PXXFYF (SEQ ID NO: 123), XXXFYFX (SEQ ID NO: 124), XXXFYFP (SEQ ID NO: 125), PXXFYFX (SEQ ID NO: 126), XPXFYFX (SEQ ID NO: 127), PXXFYFP (SEQ ID NO: 9), XPXFYFP (SEQ ID NO: 128), XXXFYFXX (SEQ ID NO: 129), PXXFYFXX (SEQ ID NO: 130), XPXFYFXX (SEQ ID NO: 131), XXXFYFPX (SEQ ID NO: 132), PXXFYFPX (SEQ ID NO: 133), or XPXFYFPX (SEQ ID NO: 134) sequence of a PP2A B subunit (e.g., a FYFP (SEQ ID NO: 3) sequence of PR70 and PR72) when bound to a PP2A core enzyme (i.e., PP2A holoenzyme). In some embodiments a small molecule agent comprises a structure that mimics the 3-dimensional structure of a (S/T)FYF (SEQ ID NO: 135), FYFX (SEQ ID NO: 111), FYFP (SEQ ID NO: 3), (S/T)FYFX (SEQ ID NO: 136), (S/T)FYFP (SEQ ID NO: 137), X(S/T)FYF (SEQ ID NO: 138), P(S/T)FYF (SEQ ID NO: 139), FYFXX, FYFPX, X(S/T)FYFX (SEQ ID NO: 140), P(S/T)FYFX (SEQ ID NO: 141), X(S/T)FYFP (SEQ ID NO: 142), P(S/T)FYFP (SEQ ID NO: 6), XX(S/T)FYF (SEQ ID NO: 143), XP(S/T)FYF (SEQ ID NO: 144), PX(S/T)FYF (SEQ ID NO: 145), XX(S/T)FYFX (SEQ ID NO: 146), XX(S/T)FYFP (SEQ ID NO: 147), PX(S/T)FYFX (SEQ ID NO: 148), XP(S/T)FYFX (SEQ ID NO: 149), PX(S/T)FYFP (SEQ ID NO: 9), XP(S/T)FYFP (SEQ ID NO: 150), XX(S/T)FYFXX (SEQ ID NO: 151), PX(S/T)FYFXX (SEQ ID NO: 152), XP(S/T)FYFXX (SEQ ID NO: 153), XX(S/T)FYFPX (SEQ ID NO: 154), PX(S/T)FYFPX (SEQ ID NO: 155), or XP(S/T)FYFPX (SEQ ID NO: 156) sequence of a PP2A B subunit (e.g., a sequence of PR70 and PR72) when bound to a PP2A core enzyme (i.e., PP2A holoenzyme). In some embodiments a small molecule agent comprises a structure that mimics the 3-dimensional structure of a H(S/T)FYF (SEQ ID NO: 157), H(S/T)FYFX (SEQ ID NO: 158), H(S/T)FYFP (SEQ ID NO: 159), XH(S/T)FYF (SEQ ID NO: 160), PH(S/T)FYF (SEQ ID NO: 161), XH(S/T)FYFX (SEQ ID NO: 162), XH(S/T)FYFP (SEQ ID NO: 163), PH(S/T)FYFX (SEQ ID NO: 164), PH(S/T)FYFP (SEQ ID NO: 165), XH(S/T)FYFXX (SEQ ID NO: 166), PH(S/T)FYFXX (SEQ ID NO: 167), XH(S/T)FYFPX (SEQ ID NO: 168), or PH(S/T)FYFPX (SEQ ID NO: 169) sequence of a PP2A B subunit (e.g., a sequence of PR70 and PR72) when bound to a PP2A core enzyme (i.e., PP2A holoenzyme).

In some embodiments a small molecule agent comprises a structure that mimics the 3-dimensional structure of a FYF (SEQ ID NO: 2) sequence of a PP2A B subunit (e.g., a FYF (SEQ ID NO: 2) sequence of PR70 and PR72) when bound to a PP2A core enzyme (i.e., PP2A holoenzyme). In some embodiments a small molecule agent comprises a structure that mimics the 3-dimensional structure of a FYFP (SEQ ID NO: 3) sequence of a PP2A B subunit (e.g., a FYFP (SEQ ID NO: 3) sequence of PR70 and PR72) when bound to a PP2A core enzyme (i.e., PP2A holoenzyme). In some embodiments a small molecule agent comprises a structure that mimics the 3-dimensional structure of a TFYFP (SEQ ID NO: 176) sequence of a PP2A B subunit (e.g., a TFYFP (SEQ ID NO: 176) sequence of PR70 and PR72) when bound to a PP2A core enzyme (i.e., PP2A holoenzyme). In some embodiments a small molecule agent comprises a structure that mimics the 3-dimensional structure of a SFYFP (SEQ ID NO: 177) sequence of a PP2A B subunit (e.g., a SFYFP (SEQ ID NO: 177) sequence of PR70 and PR72) when bound to a PP2A core enzyme (i.e., PP2A holoenzyme). In some embodiments a small molecule agent comprises a structure that mimics the 3-dimensional structure of a PSFYFP (SEQ ID NO: 7) sequence of a PP2A B subunit (e.g., a PSFYFP (SEQ ID NO: 7) sequence of PR70 and PR72) when bound to a PP2A core enzyme (i.e., PP2A holoenzyme). In some embodiments a small molecule agent comprises a structure that mimics the 3-dimensional structure of a PTFYFP (SEQ ID NO: 8) sequence of a PP2A B subunit (e.g., a PTFYFP (SEQ ID NO: 8) sequence of PR70 and PR72) when bound to a PP2A core enzyme (i.e., PP2A holoenzyme).

### Nucleic Acid Agents

Nucleic acid agents comprise one or more nucleic acids. Therapeutic nucleic acid agents can modulate, e.g., the activity of NF-κB, such as by competitively inhibiting binding of a B subunit of PP2A (e.g., PR70) to a PP2A core enzyme. In some embodiments a nucleic acid agent comprises a structure that mimics the 3-dimensional structure of a XFYF (SEQ ID NO: 110), FYFX (SEQ ID NO: 111), FYFP (SEQ ID NO: 3), XFYFX (SEQ ID NO: 112), XFYFP (SEQ ID NO: 113), XXFYF (SEQ ID NO: 114), PXFYF (SEQ ID NO: 115), FYFXX (SEQ ID NO: 116), FYFPX (SEQ ID NO: 117), XXFYFX (SEQ ID NO: 118), PXFYFX (SEQ ID NO: 119), XXFYFP (SEQ ID NO: 120), PXFYFP (SEQ ID NO: 5), XXXFYF (SEQ ID NO: 121), XPXFYF (SEQ ID NO: 122), PXXFYF (SEQ ID NO: 123), XXXFYFX (SEQ ID NO: 124), XXXFYFP (SEQ ID NO: 125), PXXFYFX (SEQ ID NO: 126), XPXFYFX (SEQ ID NO: 127), PXXFYFP (SEQ ID NO: 9), XPXFYFP (SEQ ID NO: 128), XXXFYFXX (SEQ ID NO: 129), PXXFYFXX (SEQ ID NO: 130), XPXFYFXX (SEQ ID NO: 131), XXXFYFPX (SEQ ID NO: 132), PXXFYFPX (SEQ ID NO: 133), or XPXFYFPX (SEQ ID NO: 134) sequence of a PP2A B subunit (e.g., a FYFP (SEQ ID NO: 3) sequence of PR70 and PR72) when bound to a PP2A core enzyme (i.e., PP2A holoenzyme). In some embodiments a nucleic acid agent comprises a structure that mimics the 3-dimensional structure of a (S/T)FYF (SEQ ID NO: 135), FYFX (SEQ ID NO: 111), FYFP (SEQ ID NO: 3), (S/T)FYFX (SEQ ID NO: 136), (S/T)FYFP (SEQ ID NO: 137), X(S/T)FYF (SEQ ID NO: 138), P(S/T)FYF (SEQ ID NO: 139), FYFXX, FYFPX, X(S/T)FYFX (SEQ ID NO: 140), P(S/T)FYFX (SEQ ID NO: 141), X(S/T)FYFP (SEQ ID NO: 142), P(S/T)FYFP (SEQ ID NO: 6), XX(S/T)FYF (SEQ ID NO: 143), XP(S/T)FYF (SEQ ID NO: 144), PX(S/T)FYF (SEQ ID NO: 145), XX(S/T)FYFX (SEQ ID NO: 146), XX(S/T)FYFP (SEQ ID NO: 147), PX(S/T)FYFX (SEQ ID NO: 148), XP(S/T)FYFX (SEQ ID NO: 149), PX(S/T)FYFP (SEQ ID NO: 9), XP(S/T)FYFP (SEQ ID NO: 150), XX(S/T)FYFXX (SEQ ID NO: 151), PX(S/T)FYFXX (SEQ ID NO: 152), XP(S/T)FYFXX (SEQ ID NO: 153), XX(S/T)FYFPX (SEQ ID NO: 154), PX(S/T)FYFPX (SEQ ID NO: 155), or XP(S/T)FYFPX (SEQ ID NO: 156) sequence of a PP2A B subunit (e.g., a sequence of PR70 and PR72) when bound to a PP2A core enzyme (i.e., PP2A holoenzyme). In some embodiments a nucleic acid agent comprises a structure that mimics the 3-dimensional structure of a H(S/T)FYF (SEQ ID NO: 157), H(S/T)FYFX (SEQ ID NO: 158), H(S/T)FYFP (SEQ ID NO: 159), XH(S/T)FYF (SEQ ID NO: 160), PH(S/T)FYF (SEQ ID NO: 161), XH(S/T)FYFX (SEQ ID NO: 162), XH(S/T)FYFP (SEQ ID NO: 163), PH(S/T)FYFX (SEQ ID NO: 164), PH(S/T)FYFP (SEQ ID NO: 165), XH(S/T)FYFXX (SEQ ID NO: 166), PH(S/T)FYFXX (SEQ ID NO: 167), XH(S/T)FYFPX (SEQ ID NO: 168), or PH(S/T)FYFPX (SEQ ID NO: 169) sequence of a PP2A B subunit (e.g., a sequence of PR70 and PR72) when bound to a PP2A core enzyme (i.e., PP2A holoenzyme).

In some embodiments a nucleic acid agent comprises a structure that mimics the 3-dimensional structure of a FYF (SEQ ID NO: 2) sequence of a PP2A B subunit (e.g., a FYF (SEQ ID NO: 2) sequence of PR70 and PR72) when bound to a PP2A core enzyme (i.e., PP2A holoenzyme). In some embodiments a nucleic acid agent comprises a structure that mimics the 3-dimensional structure of a FYFP (SEQ ID NO: 3) sequence of a PP2A B subunit (e.g., a FYFP (SEQ ID NO: 3) sequence of PR70 and PR72) when bound to a PP2A core enzyme (i.e., PP2A holoenzyme). In some embodiments a nucleic acid agent comprises a structure that mimics the 3-dimensional structure of a TFYFP (SEQ ID NO: 176) sequence of a PP2A B subunit (e.g., a TFYFP (SEQ ID NO: 176) sequence of PR70 and PR72) when bound to a PP2A core enzyme (i.e., PP2A holoenzyme). In some embodiments a nucleic acid agent comprises a structure that mimics the 3-dimensional structure of a SFYFP (SEQ ID NO: 177) sequence of a PP2A B subunit (e.g., a SFYFP (SEQ ID NO: 177) sequence of PR70 and PR72) when bound to a PP2A core enzyme (i.e., PP2A holoenzyme). In some embodiments a nucleic acid agent comprises a structure that mimics the 3-dimensional structure of a PSFYFP (SEQ ID NO: 7) sequence of a PP2A B subunit (e.g., a PSFYFP (SEQ ID NO: 7) sequence of PR70 and PR72) when bound to a PP2A core enzyme (i.e., PP2A holoenzyme). In some embodiments a nucleic acid agent comprises a structure that mimics the 3-dimensional structure of a PTFYFP (SEQ ID NO: 8) sequence of a PP2A B subunit (e.g., a PTFYFP (SEQ ID NO: 8) sequence of PR70 and PR72) when bound to a PP2A core enzyme (i.e., PP2A holoenzyme).

In some embodiments nucleic acid agents can be used as antisense constructs to control gene expression in cells, tissues or organs. The methodology associated with antisense techniques is well known to the skilled artisan, and is described and reviewed in Antisense Drug Technology: Principles, Strategies, and Applications, Crooke, Marcel Dekker Inc., New York (2001) In general, antisense nucleic acids are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RNAse H or RNAse L) that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example, by gene knock-out or gene knock-down experiments. Antisense technology is further described in Lavery et al., Curr. Opin. Drug Discov Devel 6 561-569 (2003), Stephens et al., Curr. Opin. Mol Ther. 5.118-122 (2003), Kurreck, Eur. J. Biochem. 270.1628-44 (2003), Dias et al, Mol Cancer Ter. 1-347-55 (2002), Chen, Methods Mol Med. 75:621-636 (2003), Wang et al., Curr Cancer Drug Targets 1.177-96 (2001), and Bennett, Antisense Nucleic Acid Drug. Dev. 12 215-24 (2002)

As antisense molecules can be used to inactivate mRNA so as to inhibit gene expression, and thus protein expression, the molecules can be used to treat a disease or disorder, such as inflammation. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein. The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev, Genet. 8: 173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the disclosure relates to isolated nucleic acid sequences, and the use of those molecules for RNA interference, for example as small interfering RNA molecules (siRNA). In one embodiment, the isolated nucleic acid sequences can be 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pn-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8: 173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siola et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23.559-565 (2006), Brummelkamp et al., Science 296. 550-553 (2002)).

Since RNAi molecules, including siRNA, miRNA and shRNA, act in a sequence-dependent manner, RNAi reagents that recognize specific nucleic acids, while not recognizing other nucleic acid sequences, can be designed. These RNAi reagents can thus recognize and destroy the target nucleic acid sequences. As with antisense reagents, RNAi reagents can be useful as therapeutic agents (i.e., for turning off disease-associated genes or disease-associated gene variants).

Delivery of RNAi can be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to RNase activity. Other chemical modifications are possible and known to those skilled in the art.

The following references provide a further summary of RNAi, and possibilities for targeting specific genes using RNAi: Kim & Rossi, Nat. Rev. Genet. 8: 173-184 (2007), Chen & Rajewsky, Nat. Rev. Genet. 8: 93-103 (2007), Reynolds, et al., Nat. Biotechnol 22 326-330 (2004), Chi et al., Proc. Natl. Acad. Sa. USA 100-6343-6346 (2003), Vickers et al., J Biol Chem. 278:7108-7118 (2003), Agami, Curr Opin. Chem. Biol. 6:829-834 (2002), Lavery, et al., Curr. Opin. Drug Discov. Devel. 6:561-569 (2003), Shi, Trends Genet. 19:9-12 (2003), Shuey et al., Drug Discov. Today 7 1040-46 (2002), McManus et al., Nat. Rev. Genet. 3.737-747 (2002), Xia et al., Nat. Biotechnol. 20.1006-10 (2002), Plasterk et al., Curr. Opin Genet. Dev. 10 562-7 (2000), Bosher et al., Nat. Cell Biol. 2:E31-6 (2000), and Hunter, Curr. Biol. 9:R440-442 (1999).

A genetic defect leading to increased predisposition or risk for development of a disease, including inflammation, or a defect causing the disease, can be corrected permanently by administering to a subject carrying the defect a nucleic acid fragment that incorporates a repair sequence that supplies the normal/wild-type nucleotide(s) at the site of the genetic defect. Such site-specific repair sequence can encompass an RNA/DNA oligonucleotide that operates to promote endogenous repair of a subject's genomic DNA. The administration of the repair sequence can be performed by an appropriate vehicle, such as a complex with polyethelamine, encapsulated in anionic liposomes, a viral vector such as an adenovirus vector, or other pharmaceutical compositions suitable for promoting intracellular uptake of the administered nucleic acid The genetic defect can then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

Double stranded RNA induced gene silencing can occur on at least three different levels: (i) transcription inactivation, which refers to RNA guided DNA or histone methylation; (ii) siRNA induced mRNA degradation; and (iii) mRNA induced transcriptional attenuation. It is generally considered that the major mechanism of RNA induced silencing (RNA interference, or RNAi) in mammalian cells is mRNA degradation. RNA interference (RNAi) is a mechanism that inhibits gene expression at the stage of translation or by hindering the transcription of specific genes. Specific RNAi pathway proteins are guided by the dsRNA to the targeted messenger RNA (mRNA), where they “cleave” the target, breaking it down into smaller portions that can no longer be translated into protein. Initial attempts to use RNAi in mammalian cells focused on the use of long strands of dsRNA. However, these attempts to induce RNAi met with limited success, due in part to the induction of the interferon response, which results in a general, as opposed to a target-specific, inhibition of protein synthesis. Thus, long dsRNA is not a viable option for RNAi in mammalian systems. Another outcome is epigenetic changes to a gene—histone modification and DNA methylation—affecting the degree the gene is transcribed.

More recently it has been shown that when short (18-30 bp) RNA duplexes are introduced into mammalian cells in culture, sequence-specific inhibition of target mRNA can be realized without inducing an interferon response. Certain of these short dsRNAs, referred to as small inhibitory RNAs (“siRNAs”), can act catalytically at sub-molar concentrations to cleave greater than 95% of the target mRNA in the cell. A description of the mechanisms for siRNA activity, as well as some of its applications are described in Provost et al., Ribonuclease Activity and RNA Binding of Recombinant Human Dicer, E.M.B.O. J., 2002 Nov. 1; 21(21): 5864-5874; Tabara et al., The dsRNA Binding Protein RDE-4 Interacts with RDE-1, DCR-1 and a DexH-box Helicase to Direct RNAi in C. elegans, Cell 2002, Jun. 28; 109(7):861-71; Ketting et al., Dicer Functions in RNA Interference and in Synthesis of Small RNA Involved in Developmental Timing in C. elegans; Martinez et al., Single-Stranded Antisense siRNAs Guide Target RNA Cleavage in RNAi, Cell 2002, Sep. 6; 110(5):563; Hutvagner & Zamore, A microRNA in a multiple-turnover RNAi enzyme complex, Science 2002, 297:2056.

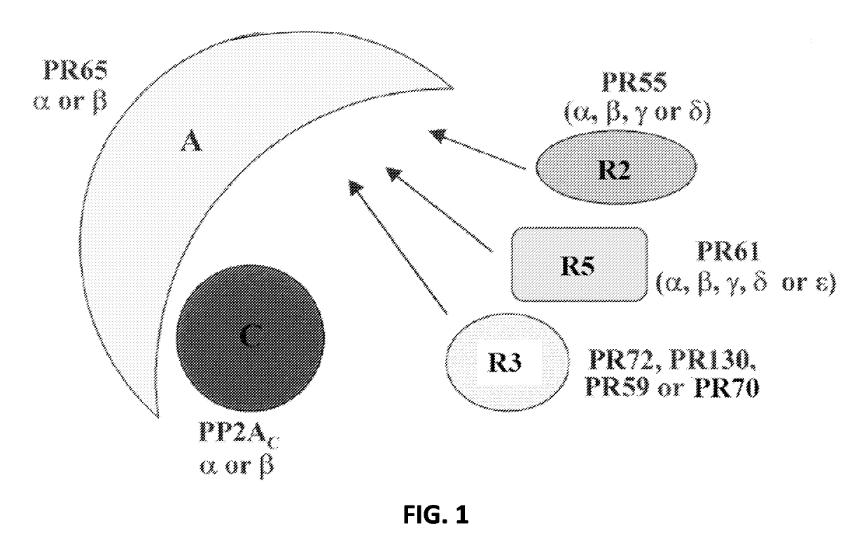

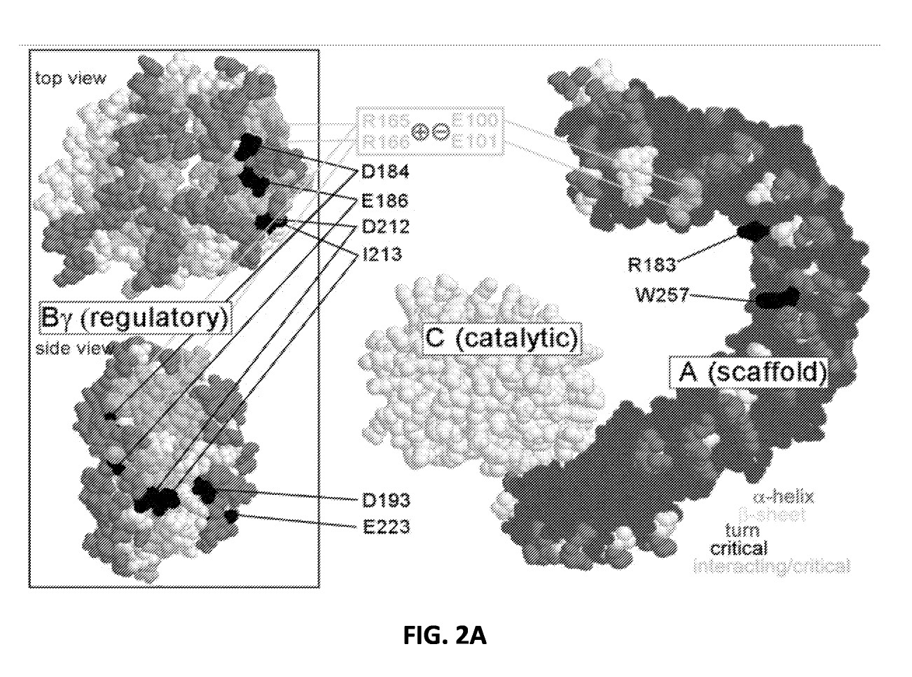

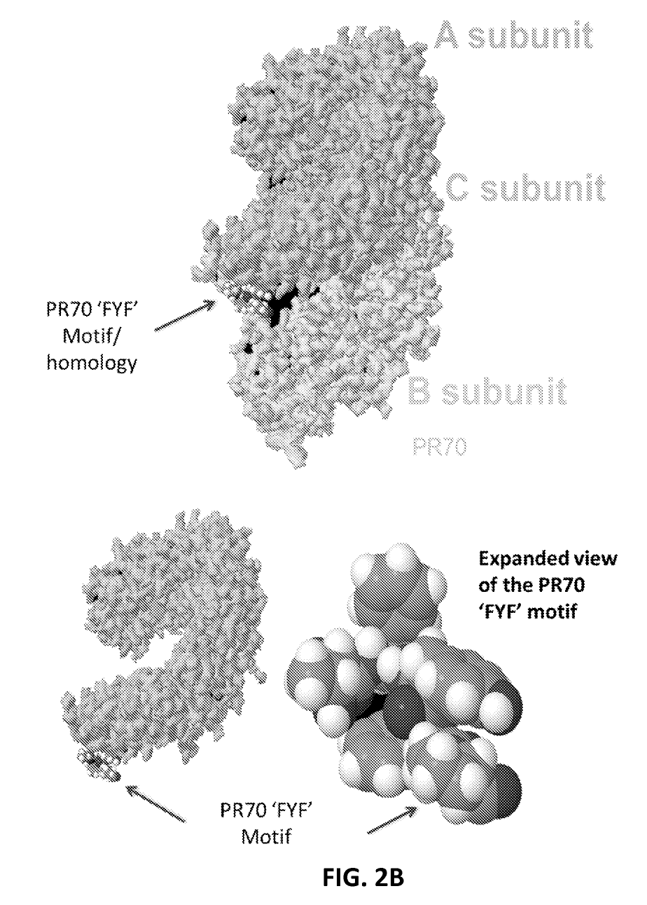

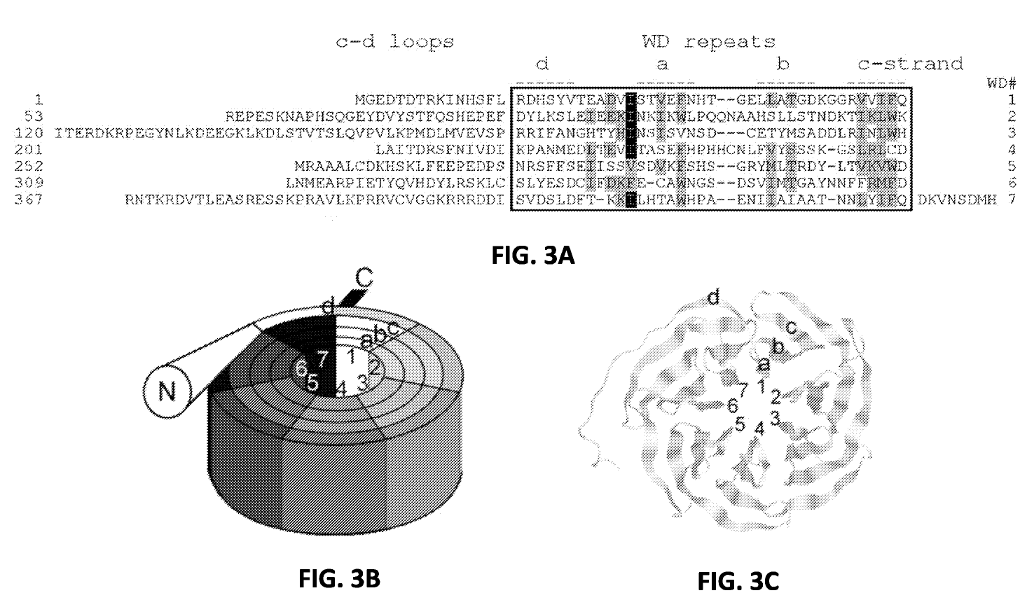

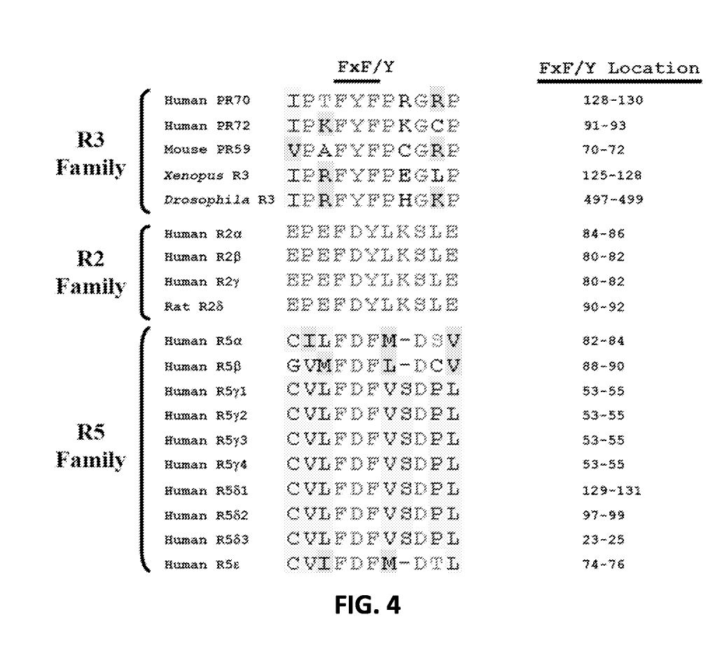

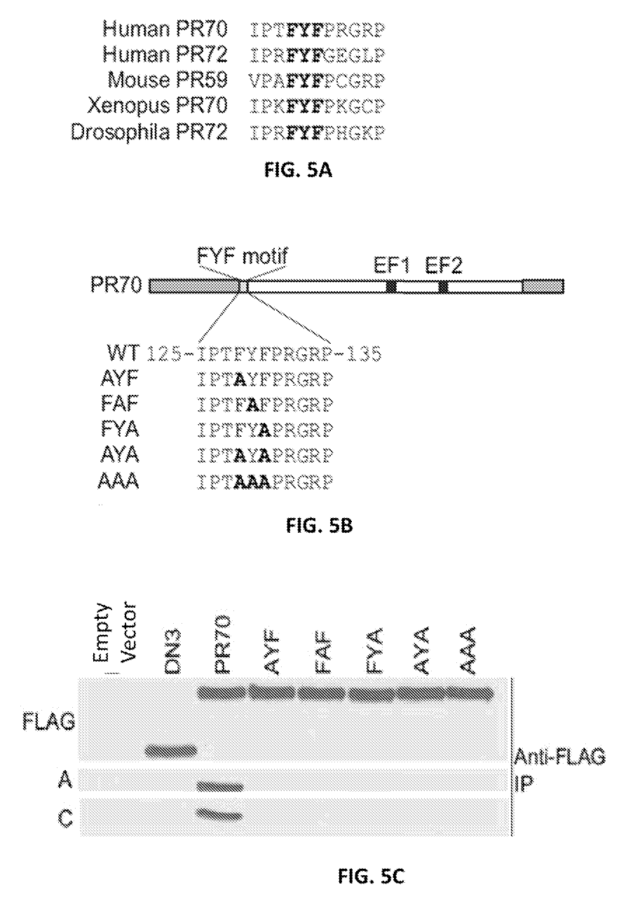

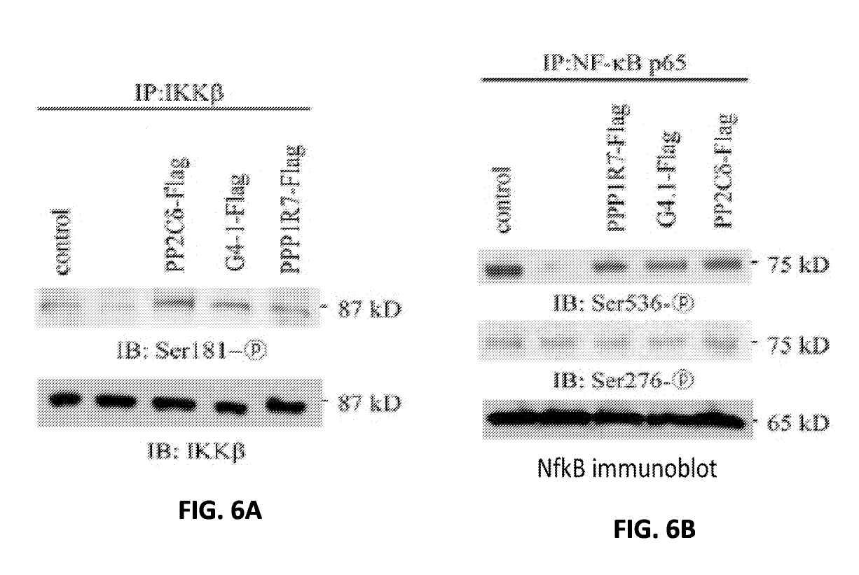

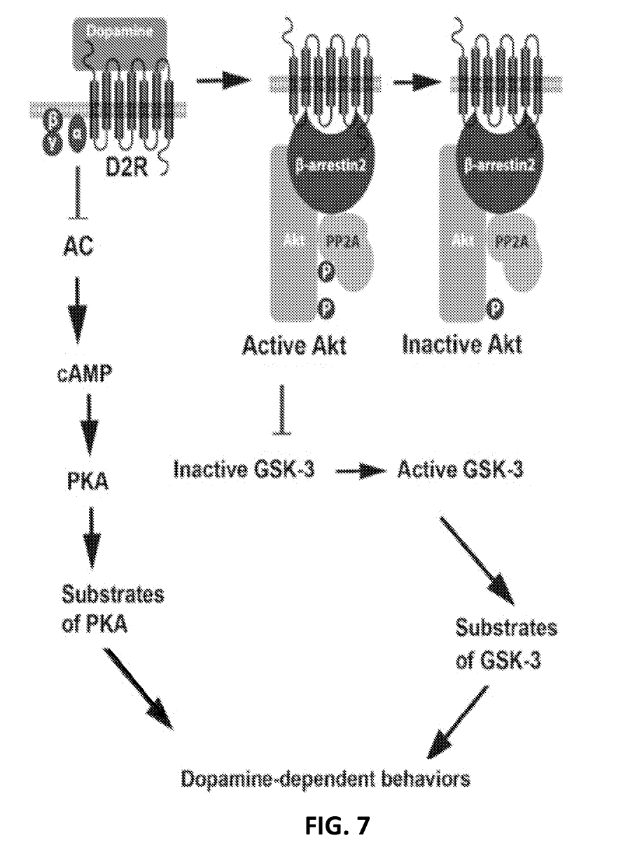

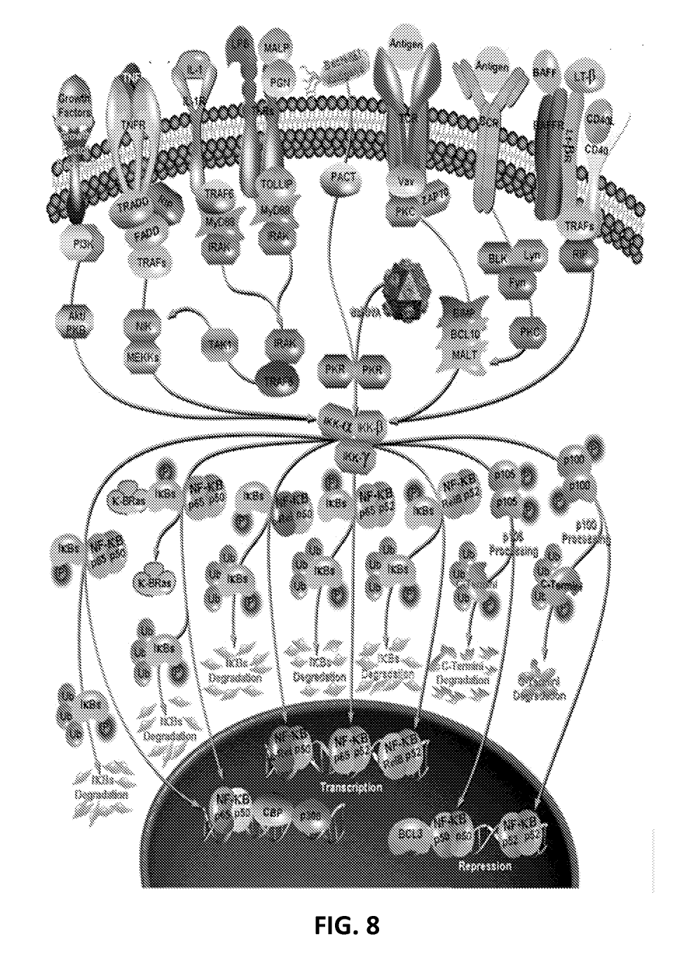

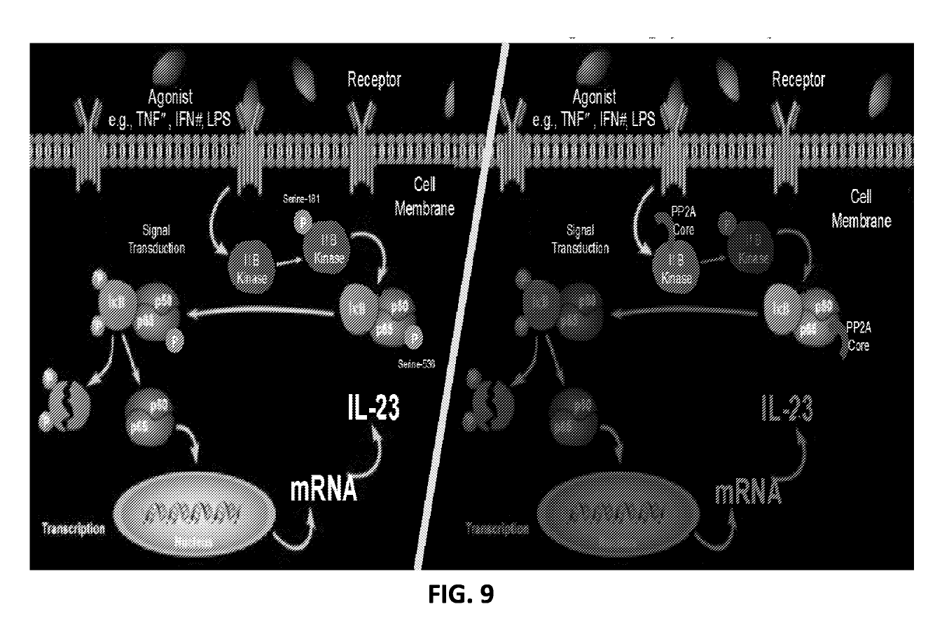

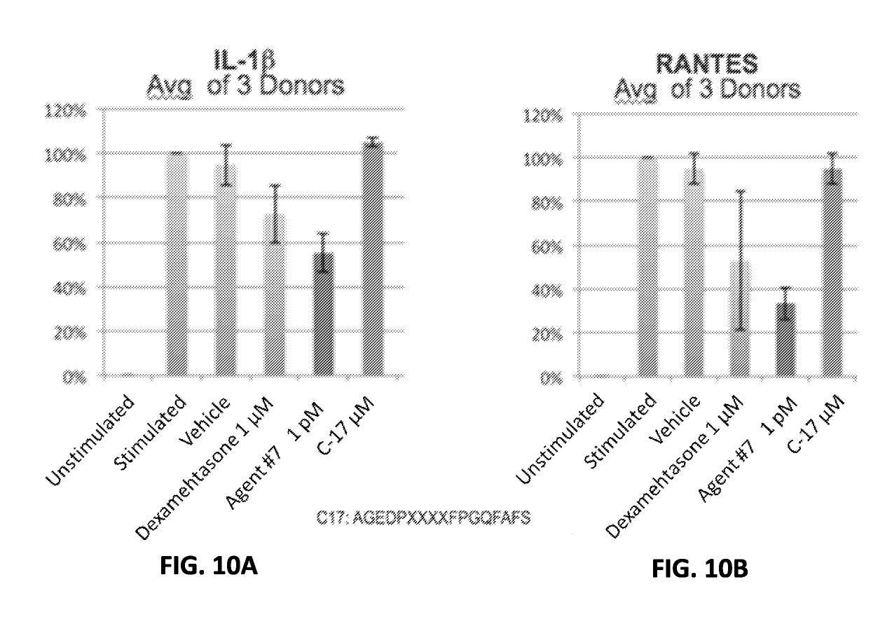

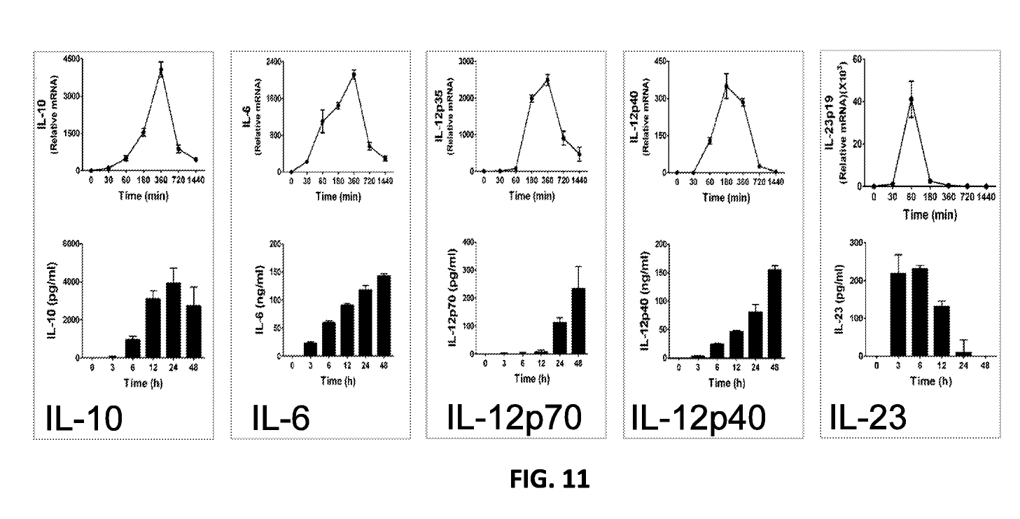

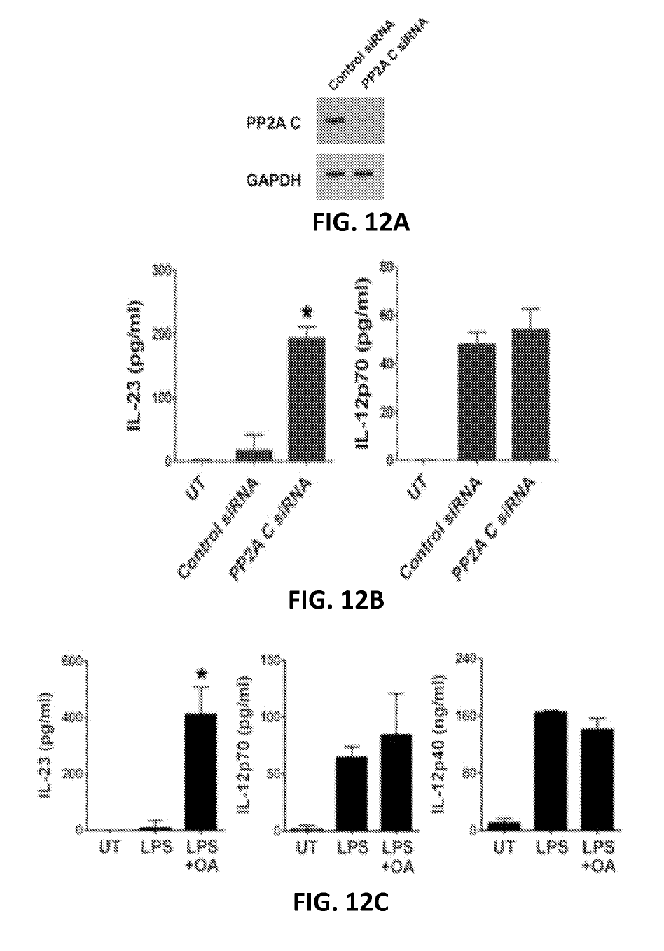

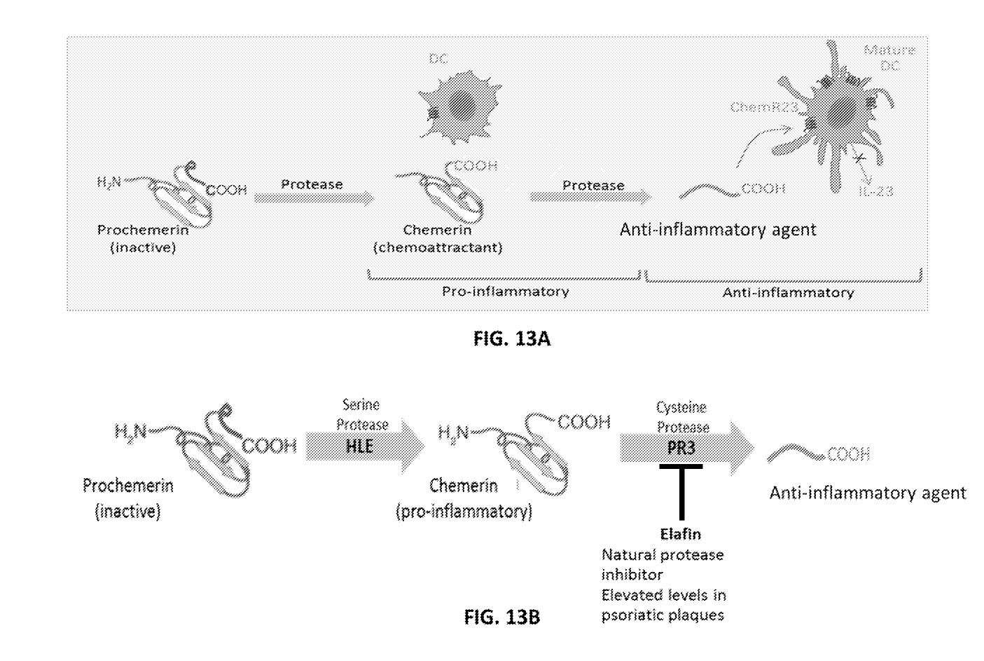

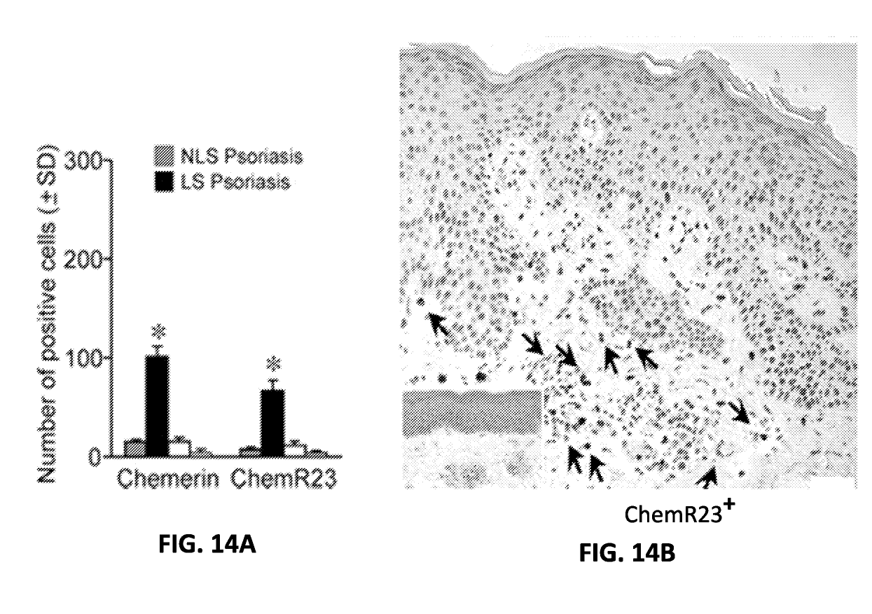

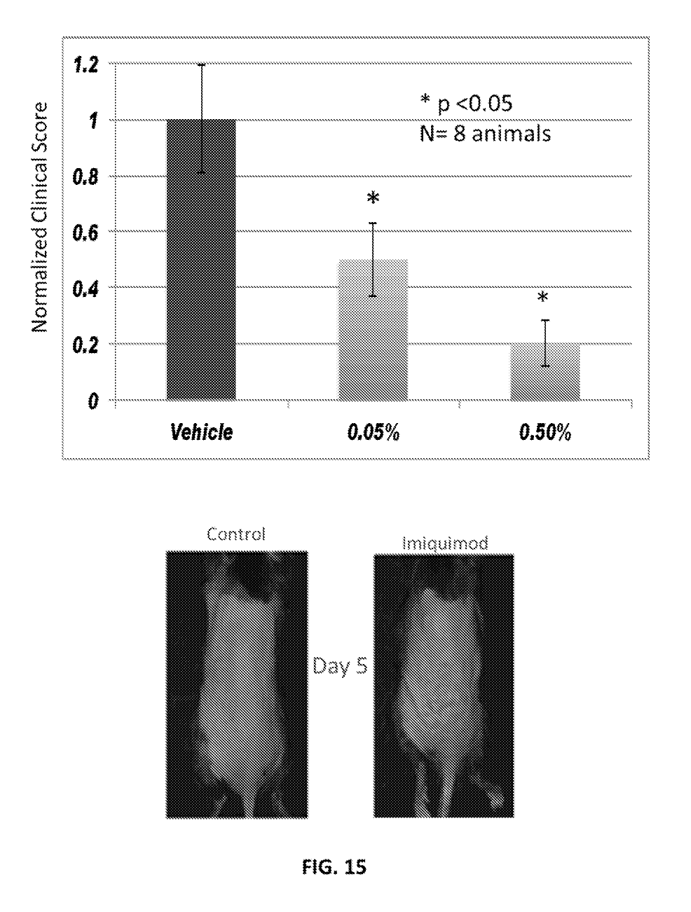

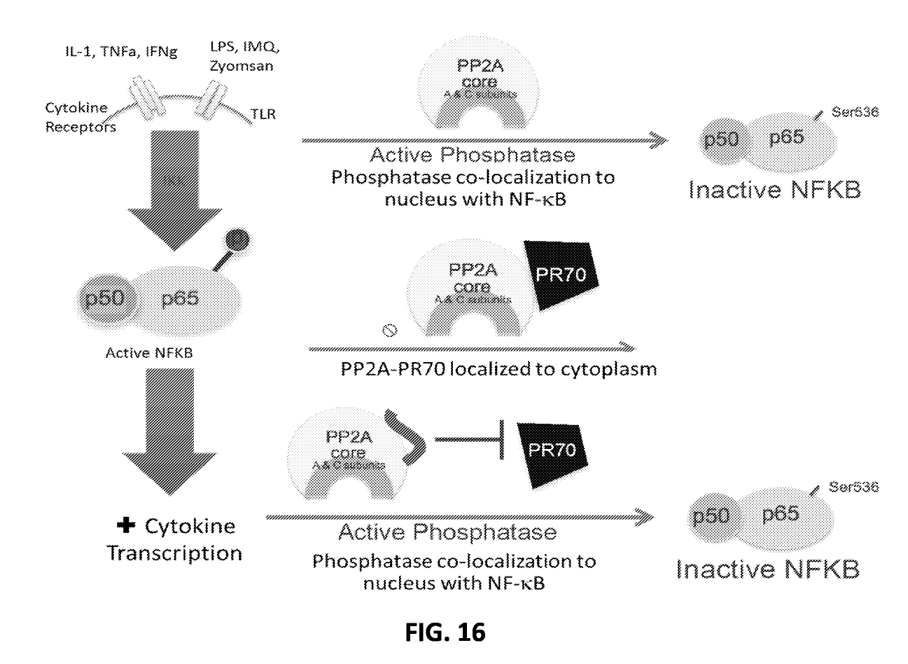

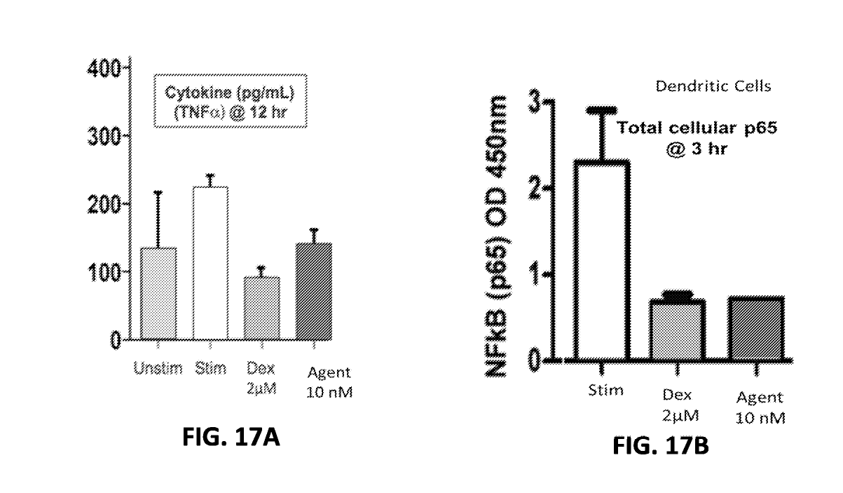

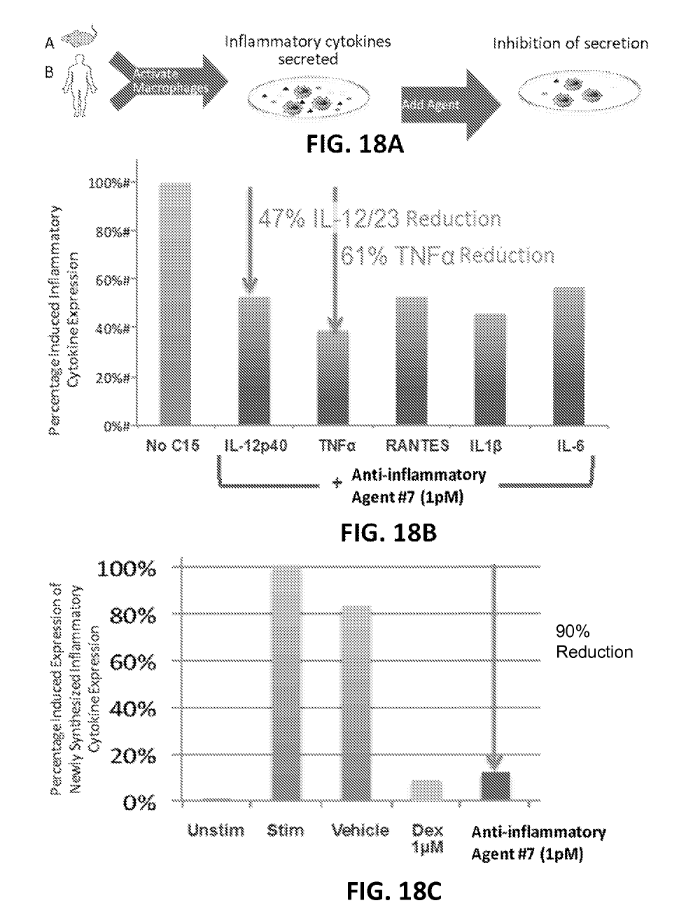

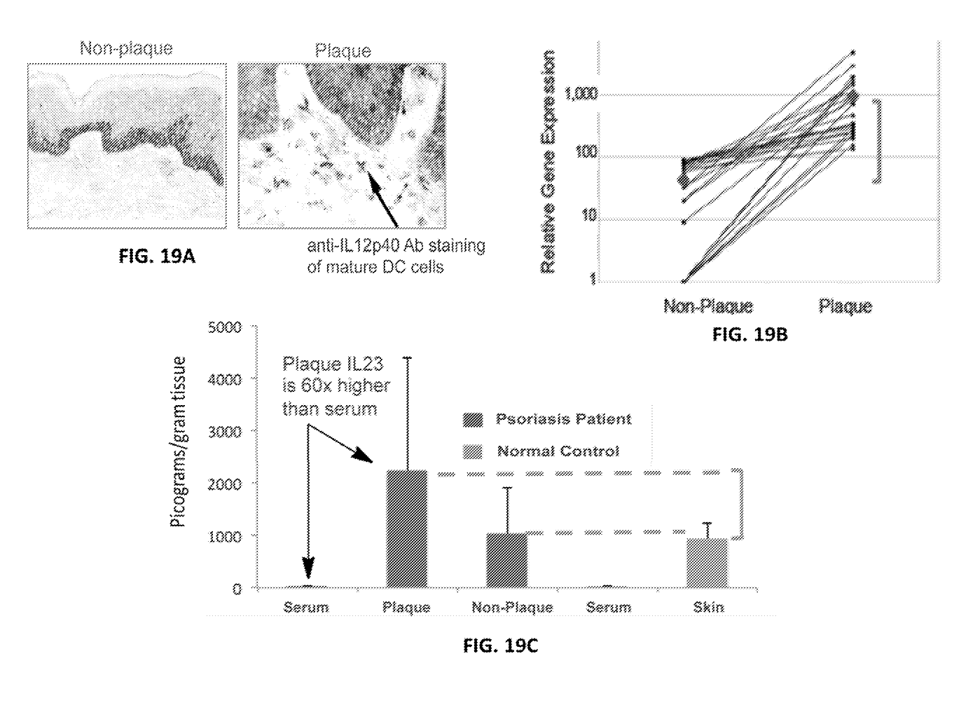

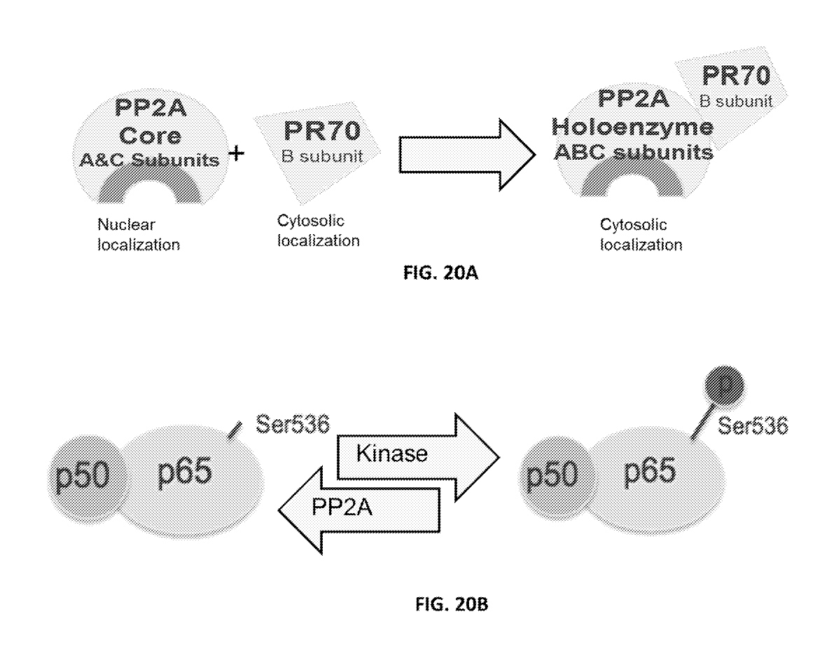

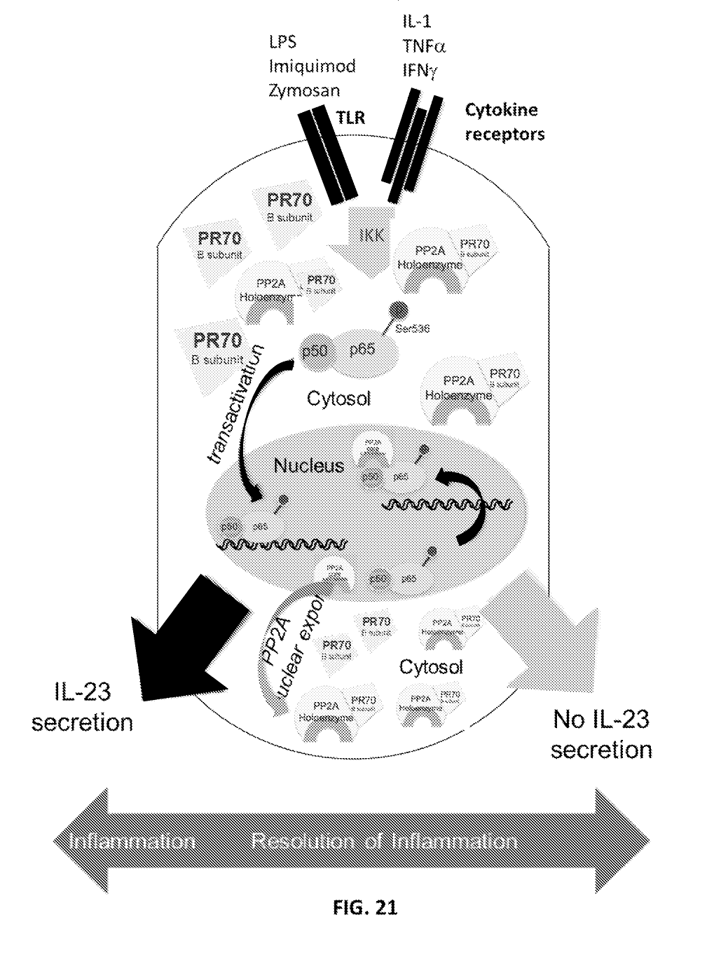

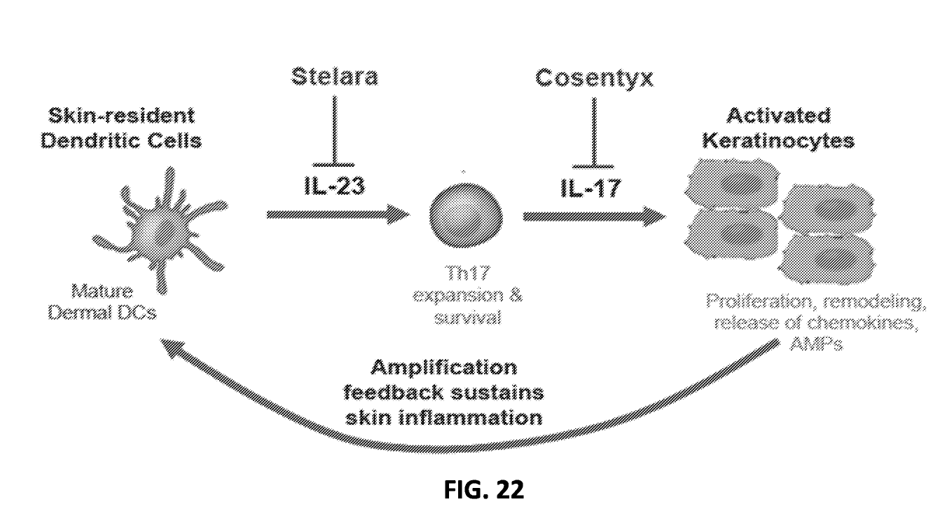

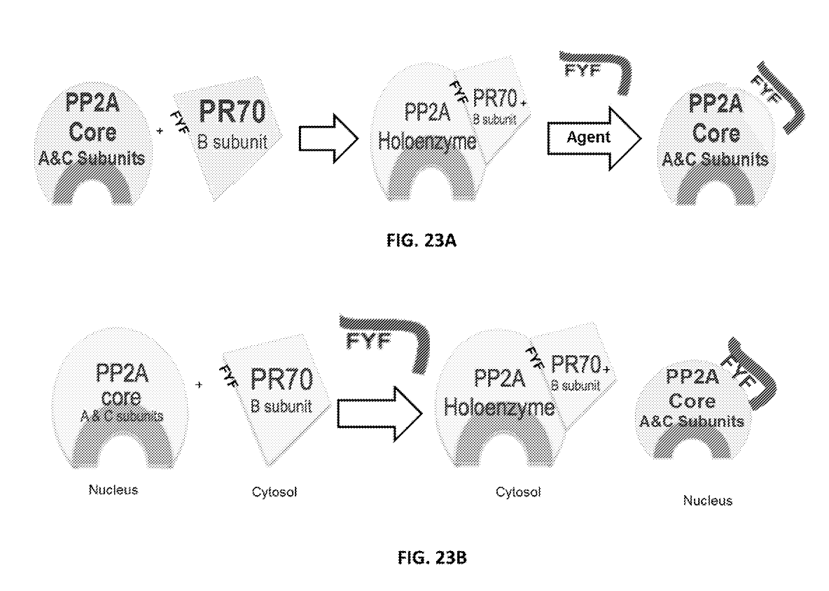

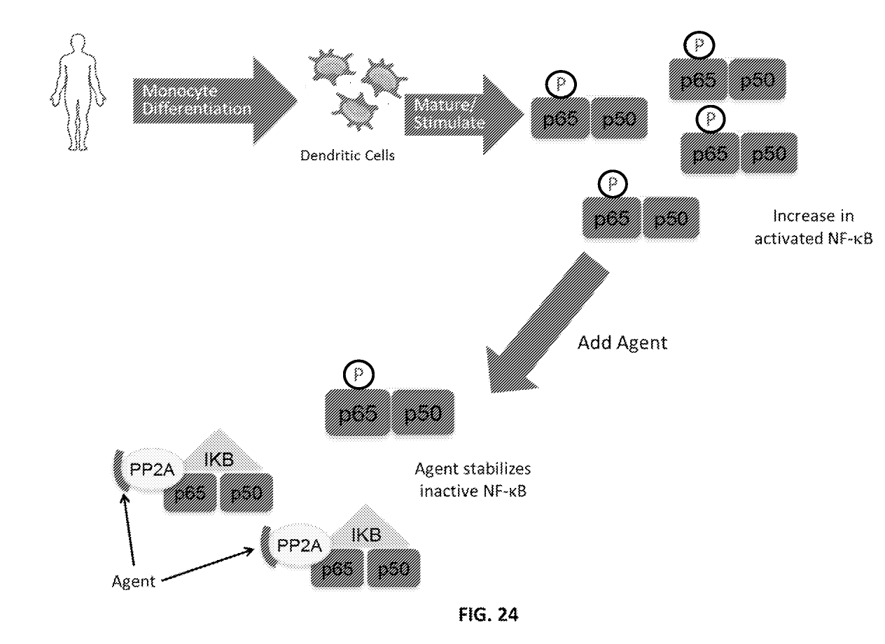

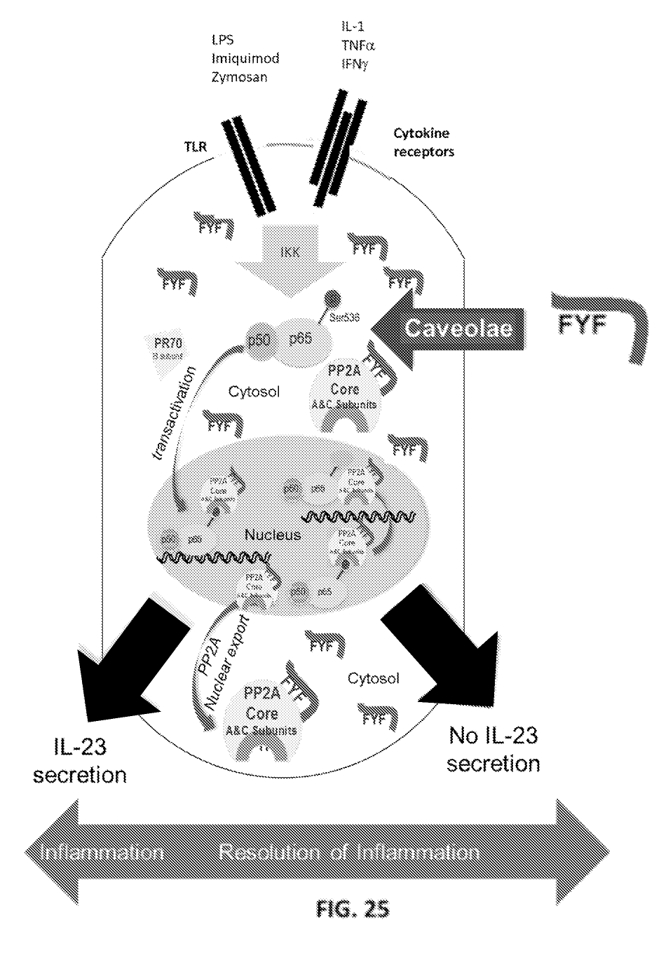

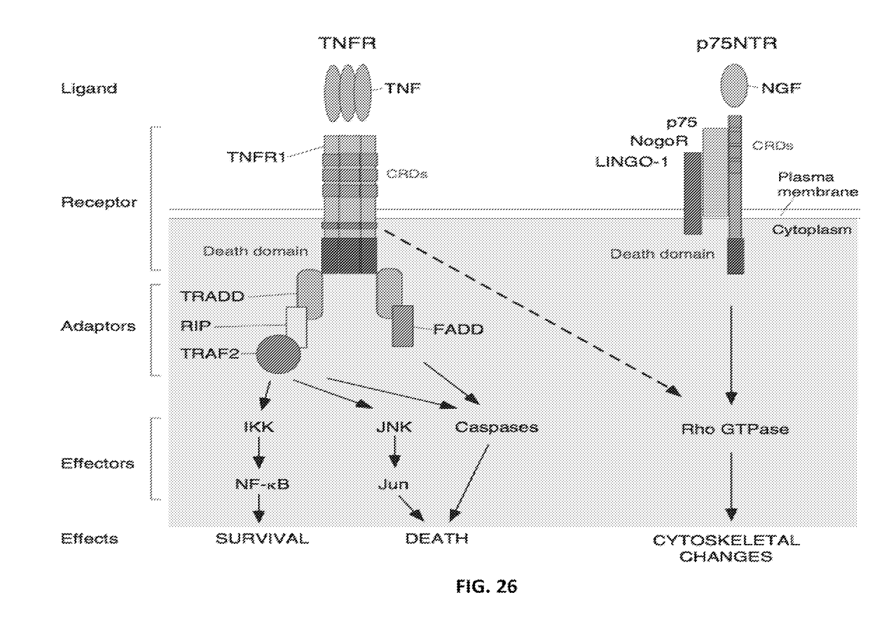

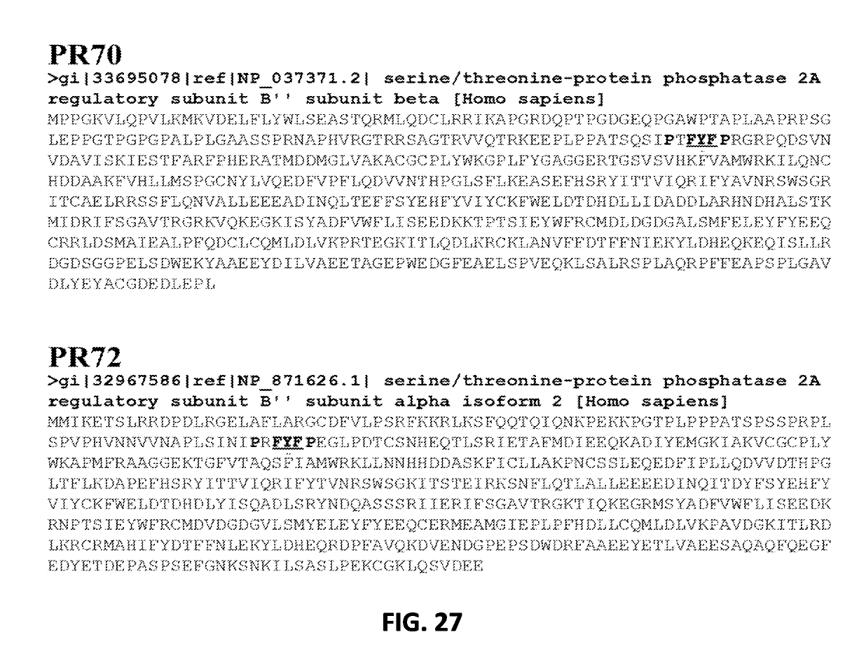

From a mechanistic perspective, introduction of long double stranded RNA into plants and invertebrate cells is broken down into siRNA by a Type III endonuclease known as Dicer. Sharp, RNA interference—2001, Genes Dev. 2001, 15:485. Dicer, a ribonuclease-III-like enzyme, processes the dsRNA into 19-23 base pair short interfering RNAs with characteristic two base 3′ overhangs. Bernstein, Caudy, Hammond, & Hannon, Role for a bidentate ribonuclease in the initiation step of RNA interference, Nature 2001, 409:363. The siRNAs are then incorporated into an RNA-induced silencing complex (RISC) where one or more helicases unwind the siRNA duplex, enabling the complementary antisense strand to guide target recognition (Nykanen, Haley, & Zamore, ATP requirements and small interfering RNA structure in the RNA interference pathway, Cell 2001, 107:309). Upon binding to the appropriate target mRNA, one or more endonucleases within the RISC cleaves the target to induce silencing. Elbashir, Lendeckel, & Tuschl, RNA interference is mediated by 21- and 22-nucleotide RNAs, Genes Dev 2001, 15:188, FIG. 1.

Generally, the antisense sequence is retained in the active RISC complex and guides the RISC to the target nucleotide sequence by means of complementary base-pairing of the antisense sequence with the target sequence for mediating sequence-specific RNA interference. It is known in the art that in some cell culture systems, certain types of unmodified siRNAs can exhibit “off target” effects. It is hypothesized that this off-target effect involves the participation of the sense sequence instead of the antisense sequence of the siRNA in the RISC complex (see for example, Schwarz et al., 2003, Cell, 115, 199-208). In this instance the sense sequence is believed to direct the RISC complex to a sequence (off-target sequence) that is distinct from the intended target sequence, resulting in the inhibition of the off-target sequence. In these double stranded nucleic acid sequences, each strand is complementary to a distinct target nucleic acid sequence. However, the off-targets that are affected by these dsRNAs are not entirely predictable and are non-specific.

The term “siRNA” refers to small inhibitory RNA duplexes that induce the RNA interference (RNAi) pathway. These molecules can vary in length (generally between 18-30 basepairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region. Small interfering RNA (siRNA), sometimes known as short interfering RNA or silencing RNA, are a class of 20-25 nucleotide-long double-stranded RNA molecules that play a variety of roles in biology.

While the two RNA strands do not need to be completely complementary, the strands should be sufficiently complementary to hybridize to form a duplex structure. In some instances, the complementary RNA strand can be less than 30 nucleotides, preferably less than 25 nucleotides in length, more preferably 19 to 24 nucleotides in length, more preferably 20-23 nucleotides in length, and even more preferably 22 nucleotides in length. The dsRNA of the present disclosure can further comprise at least one single-stranded nucleotide overhang. The dsRNA of the present disclosure can further comprise a substituted or chemically modified nucleotide. As discussed in detail below, the dsRNA can be synthesized by standard methods known in the art.

siRNA can be divided into five (5) groups including non-functional, semi-functional, functional, highly functional, and hyper-functional based on the level or degree of silencing that they induce in cultured cell lines. As used herein, these definitions are based on a set of conditions where the siRNA is transfected into said cell line at a concentration of 100 nM and the level of silencing is tested at a time of roughly 24 hours after transfection, and not exceeding 72 hours after transfection. In this context, “non-functional siRNA” are defined as those siRNA that induce less than 50% (<50%) target silencing. “Semi-functional siRNA” induce 50-79% target silencing. “Functional siRNA” are molecules that induce 80-95% gene silencing. “Highly-functional siRNA” are molecules that induce greater than 95% gene silencing. “Hyperfunctional siRNA” are a special class of molecules. For purposes of this document, hyperfunctional siRNA are defined as those molecules that: (1) induce greater than 95% silencing of a specific target when they are transfected at subnanomolar concentrations (i.e., less than one nanomolar); and/or (2) induce functional (or better) levels of silencing for greater than 96 hours. These relative functionalities (though not intended to be absolutes) can be used to compare siRNAs to a particular target for applications such as functional genomics, target identification and therapeutics.

microRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

### Antibody-Based Therapeutics

Antibody agents are also disclosed herein. Therapeutic antibody agents can modulate, e.g., the activity of NF-κB, such as by competitively inhibiting binding of a B subunit of PP2A (e.g., PR70) to a PP2A core enzyme. In some embodiments an antibody agent binds to a structure that mimics the 3-dimensional structure of subunit A of PP2A that interacts with a XFYF (SEQ ID NO: 110), FYFX (SEQ ID NO: 111), FYFP (SEQ ID NO: 3), XFYFX (SEQ ID NO: 112), XFYFP (SEQ ID NO: 113), XXFYF (SEQ ID NO: 114), PXFYF (SEQ ID NO: 115), FYFXX (SEQ ID NO: 116), FYFPX (SEQ ID NO: 117), XXFYFX (SEQ ID NO: 118), PXFYFX (SEQ ID NO: 119), XXFYFP (SEQ ID NO: 120), PXFYFP (SEQ ID NO: 5), XXXFYFXX (SEQ ID NO: 129), XPXFYFXX (SEQ ID NO: 131), XXXFYFPX (SEQ ID NO: 132), or XPXFYFPX (SEQ ID NO: 134) sequence of a PP2A B subunit (e.g., a FYFP (SEQ ID NO: 3) sequence of PR70 and PR72) when bound to a PP2A core enzyme (i.e., PP2A holoenzyme).

In some embodiments an antibody agent binds to a structure that mimics the 3-dimensional structure of subunit A of PP2A that interacts with a XFYF (SEQ ID NO: 110), FYFX (SEQ ID NO: 111), FYFP (SEQ ID NO: 3), XFYFX (SEQ ID NO: 112), XFYFP (SEQ ID NO: 113), XXFYF (SEQ ID NO: 114), PXFYF (SEQ ID NO: 115), FYFXX (SEQ ID NO: 116), FYFPX (SEQ ID NO: 117), XXFYFX (SEQ ID NO: 118), PXFYFX (SEQ ID NO: 119), XXFYFP (SEQ ID NO: 120), PXFYFP (SEQ ID NO: 5), XXXFYF (SEQ ID NO: 121), XPXFYF (SEQ ID NO: 122), PXXFYF (SEQ ID NO: 123), XXXFYFX (SEQ ID NO: 124), XXXFYFP (SEQ ID NO: 125), PXXFYFX (SEQ ID NO: 126), XPXFYFX (SEQ ID NO: 127), PXXFYFP (SEQ ID NO: 9), XPXFYFP (SEQ ID NO: 128), XXXFYFXX (SEQ ID NO: 129), PXXFYFXX (SEQ ID NO: 130), XPXFYFXX (SEQ ID NO: 131), XXXFYFPX (SEQ ID NO: 132), PXXFYFPX (SEQ ID NO: 133), or XPXFYFPX (SEQ ID NO: 134) sequence of a PP2A B subunit (e.g., a FYFP (SEQ ID NO: 3) sequence of PR70 and PR72) when bound to a PP2A core enzyme (i.e., PP2A holoenzyme). In some embodiments an antibody agent binds to a structure that mimics the 3-dimensional structure of subunit A of PP2A that interacts with a (S/T)FYF (SEQ ID NO: 135), FYFX (SEQ ID NO: 111), FYFP (SEQ ID NO: 3), (S/T)FYFX (SEQ ID NO: 136), (S/T)FYFP (SEQ ID NO: 137), X(S/T)FYF (SEQ ID NO: 138), P(S/T)FYF (SEQ ID NO: 139), FYFXX, FYFPX, X(S/T)FYFX (SEQ ID NO: 140), P(S/T)FYFX (SEQ ID NO: 141), X(S/T)FYFP (SEQ ID NO: 142), P(S/T)FYFP (SEQ ID NO: 6), XX(S/T)FYF (SEQ ID NO: 143), XP(S/T)FYF (SEQ ID NO: 144), PX(S/T)FYF (SEQ ID NO: 145), XX(S/T)FYFX (SEQ ID NO: 146), XX(S/T)FYFP (SEQ ID NO: 147), PX(S/T)FYFX (SEQ ID NO: 148), XP(S/T)FYFX (SEQ ID NO: 149), PX(S/T)FYFP (SEQ ID NO: 9), XP(S/T)FYFP (SEQ ID NO: 150), XX(S/T)FYFXX (SEQ ID NO: 151), PX(S/T)FYFXX (SEQ ID NO: 152), XP(S/T)FYFXX (SEQ ID NO: 153), XX(S/T)FYFPX (SEQ ID NO: 154), PX(S/T)FYFPX (SEQ ID NO: 155), or XP(S/T)FYFPX (SEQ ID NO: 156) sequence of a PP2A B subunit (e.g., a sequence of PR70 and PR72) when bound to a PP2A core enzyme (i.e., PP2A holoenzyme). In some embodiments an antibody agent binds to a structure that mimics the 3-dimensional structure of subunit A of PP2A that interacts with a H(S/T)FYF (SEQ ID NO: 157), H(S/T)FYFX (SEQ ID NO: 158), H(S/T)FYFP (SEQ ID NO: 159), XH(S/T)FYF (SEQ ID NO: 160), PH(S/T)FYF (SEQ ID NO: 161), XH(S/T)FYFX (SEQ ID NO: 162), XH(S/T)FYFP (SEQ ID NO: 163), PH(S/T)FYFX (SEQ ID NO: 164), PH(S/T)FYFP (SEQ ID NO: 165), XH(S/T)FYFXX (SEQ ID NO: 166), PH(S/T)FYFXX (SEQ ID NO: 167), XH(S/T)FYFPX (SEQ ID NO: 168), or PH(S/T)FYFPX (SEQ ID NO: 169) sequence of a PP2A B subunit (e.g., a sequence of PR70 and PR72) when bound to a PP2A core enzyme (i.e., PP2A holoenzyme).

In some embodiments an antibody agent binds to a structure that mimics the 3-dimensional structure of subunit A of PP2A that interacts with a FYF (SEQ ID NO: 2) sequence of a PP2A B subunit (e.g., a FYF (SEQ ID NO: 2) sequence of PR70 and PR72) when bound to a PP2A core enzyme (i.e., PP2A holoenzyme). In some embodiments an antibody agent binds to a structure that mimics the 3-dimensional structure of subunit A of PP2A that interacts with a FYFP (SEQ ID NO: 3) sequence of a PP2A B subunit (e.g., a FYFP (SEQ ID NO: 3) sequence of PR70 and PR72) when bound to a PP2A core enzyme (i.e., PP2A holoenzyme). In some embodiments an antibody agent binds to a structure that mimics the 3-dimensional structure of subunit A of PP2A that interacts with a TFYFP (SEQ ID NO: 176) sequence of a PP2A B subunit (e.g., a TFYFP (SEQ ID NO: 176) sequence of PR70 and PR72) when bound to a PP2A core enzyme (i.e., PP2A holoenzyme). In some embodiments an antibody agent binds to a structure that mimics the 3-dimensional structure of subunit A of PP2A that interacts with a SFYFP (SEQ ID NO: 177) sequence of a PP2A B subunit (e.g., a SFYFP (SEQ ID NO: 177) sequence of PR70 and PR72) when bound to a PP2A core enzyme (i.e., PP2A holoenzyme). In some embodiments an antibody agent binds to a structure that mimics the 3-dimensional structure of subunit A of PP2A that interacts with a PSFYFP (SEQ ID NO: 7) sequence of a PP2A B subunit (e.g., a PSFYFP (SEQ ID NO: 7) sequence of PR70 and PR72) when bound to a PP2A core enzyme (i.e., PP2A holoenzyme). In some embodiments an antibody agent binds to a structure that mimics the 3-dimensional structure of subunit A of PP2A that interacts with a PTFYFP sequence of a PP2A B subunit (e.g., a PTFYFP (SEQ ID NO: 8) sequence of PR70 and PR72) when bound to a PP2A core enzyme (i.e., PP2A holoenzyme).

The present disclosure embodies agents that modulate a peptide sequence or RNA expressed from a gene associated with inflamation. In some embodiments, the agents of the disclosure are antibody-based agents. The antibody-based agents in any suitable form of an antibody e.g., monoclonal, polyclonal, or synthetic, can be utilized in the therapeutic methods disclosed herein. The antibody-based agents include any target-binding fragment of an antibody and also peptibodies, which are engineered therapeutic molecules that can bind to human drug targets and contain peptides linked to the constant domains of antibodies. In one embodiment, the antibody agents are humanized antibodies. Methods for humanizing antibodies are well known in the art. In another embodiment, the therapeutic antibodies comprise antibodies conjugated to another agent or agents, for example, a cytotoxic compound.

The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the disclosure is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)2 fragments which can be generated by treating the antibody with an enzyme such as pepsin. The disclosure provides polyclonal and monoclonal antibodies that bind to a polypeptide of the disclosure. The term “monoclonal antibody” refers to a population of antibody molecules that contain only one species of an antigen binding site capable of immunoreacting with a particular epitope of a polypeptide of the disclosure (e.g., PP2A). A monoclonal antibody composition thus typically displays a single binding affinity for a particular polypeptide of the disclosure with which it immunoreacts.

Polyclonal antibodies can be prepared as described above by immunizing a suitable subject with a desired immunogen, e.g., polypeptide of the disclosure or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybndoma technique (Cole et al., Monoclonal Antibodies and Cancer Therapy, Alan R. Liss (1985) Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the disclosure.

Any of the many well-known protocols used for fusing lymphocytes and immortalized cell lines can be applied for the purpose of generating a monoclonal antibody to a polypeptide of the disclosure (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful. Alternative to preparing monoclonal antibody-secreting hybridomas, a monoclonal antibody to a polypeptide (e.g. PP2A) of the disclosure can be identified and isolated by screening a recombinant combinatorial immunoglobulin library (e.g., an antibody phage display library) with the polypeptide (e.g., PP2A) to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP^{a} Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619, WO 91/17271, WO 92/20791, WO 92/15679; WO 93/01288, WO 92/01047, WO 92/09690, and WO 90/02809; Fuchs et al., Bio/Technology 9: 1370-1372 (1991); Hay et al., Hum. Antibod. Hybndomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the disclosure. Such chimeric and humanized monoclonal antibodies can be produced by recombinant DNA techniques known in the art.

In some embodiments, antibody agents are useful for inhibiting NF-κB function, for example by blocking the binding of PP2A to a binding molecule or partner (e.g. PR70 or PR72). Such uses can also be applied in a therapeutic context in which treatment involves inhibiting a protein's function. An antibody can, for example, be used to block or competitively inhibit binding, thereby modulating (i.e., agonizing or antagonizing) the activity of the protein. Antibodies can be prepared against specific protein fragments or epitopes containing sites, such as amino acids or sequences of amino acids required for a specific function (e.g. inhibition of binding of an FYF (SEQ ID NO: 2) motif of a B subunit of PP2A to an A subunit of PP2A).

The present disclosure also embodies the use of any pharmacologic agent that can be conjugated to an antibody or an antibody binding fragment, and delivered in active form. Examples of such agents include cytotoxins, radioisotopes, hormones such as a steroid, anti-metabolites such as cytosines, and chemotherapeutic agents. Other embodiments can include agents such as a coagulant, a cytokine, growth factor, bacterial endotoxin or a moiety of bacterial endotoxin. The targeting antibody-based agent directs the toxin to, and thereby selectively modulates the cell expressing the targeted surface receptor. In some embodiments, therapeutic antibodies employ cross-linkers that provide high in vivo stability (Thorpe et al., Cancer Res., 48:6396, 1988). In any event, it is proposed that agents such as these can, if desired, be successfully conjugated to antibodies or antibody binding fragments, in a manner that will allow their targeting, internalization, release or presentation as required using known conjugation technology. For administration in vivo, for example, an antibody can be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof can be increased by pegylation through conjugation to polyethylene glycol.

Antibodies can be used in non-therapeutic methods as well. In some embodiments, antibody agents of the disclosure (e.g., a monoclonal antibody) can be used to isolate a polypeptide by standard techniques, such as affinity chromatography or immunoprecipitation. A polypeptide-specific antibody can facilitate the purification of natural polypeptide from cells and of recombinantly produced polypeptides expressed in host cells. Moreover, an antibody specific for a polypeptide can be used to detect the polypeptide (e.g., in a cellular lysate, cell supernatant, or tissue sample) in order to evaluate the abundance and pattern of expression of the polypeptide. Antibodies can be used diagnostically, prognostically, or theranostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotnazinylamine fluorescein, dansyl chloride or phycoerythnn; an example of a luminescent material includes luminol; examples of bioluminescent materials include luciferase, luciferin, and aequorin, and examples of suitable radioactive material include ¹²⁵I, ¹³¹I, ³⁵S or ³H. Antibodies can also be useful in pharmacogenomic analysis. In such embodiments, antibodies can be used to identify individuals that require modified treatment modalities.

In some embodiments, antibodies are useful as screening tools for evaluating proteins in conjunction with analysis by electrophoretic mobility, isoelectric point, tryptic or other protease digest, or for use in other physical assays known to those skilled in the art. Antibodies can also be used in tissue typing.

Subcellular localization of proteins can also be determined using antibodies, and can be applied to assess aberrant or agent induced modulations to subcellular localization of a protein in cells in various tissues (e.g. NF-κB). Such use can be applied in genetic testing, but also in monitoring a particular treatment modality. For example, antibodies can be used to monitor therapeutic efficacy.

### III. Identification of Agents

Provided herein in some embodiments are agents that bind to PP2A core enzyme. In some embodiments, the binding of the agent to PP2A core enzyme prevents or displaces the binding of the B regulatory subunit. In some embodiments, the active agents are identified through competitive binding experiments. For example, a labeled agent, such as a peptide derived from a PP2A B regulatory subunit (e.g., the B regulatory subunits PR70 or PR72) labeled by means common in the pharmaceutical industry (e.g., radio label, fluorescent label, stable isotopic label, etc.) is allowed to bind to a PP2A core enzyme in the presence of test agents. In some embodiments, active agents are competitive antagonists of the binding of labeled C15 or B regulatory protein to a PP2A core enzyme. In some embodiments, active agents are competitive agonists of the binding of labeled C15 or B regulatory protein to the core PP2A. In some embodiments, active agents are competitive antagonists of the binding of labeled human PR70 to the core PP2A. In some embodiments, active agents are competitive agonists of the binding of labeled or human PR70 to the core PP2A. In some embodiments, active agents are competitive antagonists of the binding of labeled human PR72 to the core PP2A. In some embodiments, active agents are competitive agonists of the binding of labeled or human PR72 to the core PP2A.

In some embodiments, agents that bind to the PP2A core enzyme are assessed in vitro for their ability to control or reduce the phosphorylation of NF-κB, IKKβ, β-arrestin, Akt or other proteins in the Classical NF-κB pathway in cells stimulated with Chemerin, TNFα, IFNγ, LPS, Zymosan, IL1 or other stimulants. In some embodiments, agents that bind to the PP2A core enzyme are assessed in vitro for their ability to control or reduce the subcellular localization of the PP2A core enzyme. In some embodiments, agents that bind to the PP2A core enzyme are assessed in vitro for their ability to control or reduce the subcellular localization of the PP2A holoenzyme.

In some embodiments, compounds that bind to PP2A are assessed for the ability to stabilize complexes of PP2A with proteins in the NF-κB pathway or proteins that regulate the NF-κB pathway including but not limited to: NF-κB, IKKβ, β-arrestin. In some embodiments, the active agents stabilize complexes of PP2A with these proteins and prevent and/or reverse activation of these proteins via phosphorylation by kinases.

In some embodiments, in vitro analysis selects for agents that reduce or inhibit expression of reporter proteins, chemokines and/or cytokines linked to disease, for example, the inflammatory cytokines, IL1, IL-2, IL-4, IL-6, IL-8, IL-12, 11-17, IL-23, and TNFα. In certain embodiments, active agents are useful in reduction of message and protein levels for IL-1, IL-2, IL-3, IL-4, IL-5, IL-6, IL-7, IL-8, IL-9, IL-11, IL-12, IL-13, IL-14, IL-15, IL-16, IL-17, IL-18, IL-19, IL-20, IL-21, IL-22, IL-23, IL-24, 11-25, IL-26, IL-27, IL-28, IL-29, IL-30, as well as TNF family member, IFN family member, MCP-1, and MIP-1.

In some embodiments, an agent, such as a PR70 peptide, reduces the levels of IL-1β and RANTES in human macrophages stimulated with IFNγ and LPS. In some embodiments, active agents are competitive antagonists of human C15 peptide binding to PP2A core enzyme, PP2A holoenzyme. In some embodiments, active agents are competitive antagonists of human PR70 binding to PP2A core enzyme, PP2A holoenzyme. In some embodiments, active agents are competitive antagonists of human PR72 binding to PP2A core enzyme. These agents may be peptides, nonpeptides, peptide mimics and peptide/nonpeptide hybrids. In some instances, the agent is a nucleic acid, such as an RNA or DNA. In some embodiments, the agent is an antibody. In some embodiments, the agent is a small molecule. In some embodiments, the agent does not reduce levels of IL-10. In some embodiments, human C15 peptide reduces IL-10 to a lesser extent than it reduces IL-23 or other inflammatory cytokines. In some embodiments, the agent increases levels of IL-10.

Analogs of C15 are particularly useful and are listed below. In some embodiments, the peptides are analogs of C15. In some embodiments, the peptides are 10-30 amino acids in length. In certain embodiments, active agents described herein are amino acid sequences, nucleic acids, proteins, or small molecules that bind to PP2A and/or inhibit activation of classical NF-κB pathway. In certain embodiments, active agents described herein are amino acid sequences, nucleic acids, proteins, or small molecules that bind to PP2A and negatively regulate NF-κB gene expression through a p65 trans activation-dependent, IκB-independent pathway, resulting in the differential regulation of IL-23 p19 and p40 expression.

In some instances, the active agents also contain a cell localization signal, which are known to facilitate peptide or compound penetration into the cell. In some instances, the active agents also contain a nuclear localization signal, which are known to facilitate peptide or compound penetration into the nucleus. For example, in some instances, the active agents also contain amino acid sequences which are known to facilitate peptide or compound penetration into the cell. For example, in some instances, the active agents also contain amino acid sequences which are known to facilitate peptide or compound penetration into a targeted subset of cells expressing a particular cell surface antigen. For example, in some instances, the active agents also contain amino acid sequences which are known to facilitate peptide or compound localization to the nucleus of the cell. Agents are administered as pharmaceutical formulations by different routes of administration including: topically as gels, lotions, creams, ointments, drops, spray, mist, microparticles, or nanoparticles; by injection, iv, sc, ip or other routes of injection; orally as a pill, solution, powder, capsule, gel; inhaled as a powder, mist, aerosol, microparticle, nanoparticle.

A variety of different agents may be screened by the above methods. Candidate agents encompass numerous chemical classes including, but not limited to, peptides, polynucleotides, and organic molecules (e.g., small organic compounds having a molecular weight of more than 50 and less than about 2,500 Daltons). Candidate agents can comprise functional groups for structural interaction with target analytes, such as hydrogen bonding, and can include at least one or at least two of an amine, carbonyl, hydroxyl or carboxyl group. The candidate agents can comprise cyclical carbon or heterocyclic structures and/or aromatic or polyaromatic structures substituted with one or more functional groups. Candidate agents can be biomolecules including peptides, saccharides, fatty acids, steroids, purines, pyrimidines, derivatives, structural analogs or combinations thereof. Candidate agents can be obtained from a wide variety of sources including libraries of synthetic or natural compounds. For example, numerous means are available for random and directed synthesis of a wide variety of organic compounds and biomolecules, including expression of randomized polynucleotides and polypeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, acidification, etc. to produce structural analogs.

Furthermore, arrays may also be used in a method for screening agents. In some embodiments, a candidate agent is screened directly for its ability to bind or otherwise interact with PP2A core enzyme on the array. In some embodiments, a candidate agent is screened directly for its ability to bind or otherwise interact with PR70 on the array. In some embodiments, a candidate agent is screened directly for its ability to bind or otherwise interact with PR72 on the array. Alternatively, a plurality of potential agents may be screened in parallel for their ability to bind or otherwise interact with PP2A core enzyme, or a B subunit of PP2A. The screening process may involve assaying for the interaction, such as binding, of at least one agent with PP2A core enzyme and/or a B subunit of PP2A on the array.

In some embodiments, an array comprising a plurality of candidate agents is screened directly for their ability to bind or otherwise interact with PP2A core enzyme. In some embodiments, an array comprising a plurality of candidate agents is screened directly for their ability to bind or otherwise interact with PR70 on the array. In some embodiments, an array comprising a plurality of candidate agents is screened directly for their ability to bind or otherwise interact with PR72 on the array. Alternatively, a plurality of potential PP2A A or C subunits may be screened in parallel for their ability to bind or otherwise interact with one or more test agents. The screening process may involve assaying for the interaction, such as binding, of at least one agent with a PP2A A subunit and/or a PP2A C subunit on the array.

An array can be a high-density array. A high-density array can comprise tens, hundreds, thousands, tens-of-thousands or hundreds-of-thousands of target analytes and/or address polynucleotides. The density of microspots of an array may be at least about 1/cm² or at least about 10/cm², up to about 500/cm² or up to about 1,000/cm². In certain embodiments, the density of all the microspots on the surface of the substrate may be up to about 400/cm², up to about 300/cm², up to about 200/cm², up to about 100/cm², up to about 90/cm², up to about 80/cm², up to about 70/cm², up to about 60/cm², or up to about 50/cm². For example, an array can comprise at least 50, 60, 70, 80, 90, 100, 150, 200, 250, 300, 350, 400, 450, 500, 600, 700, 800, 900, or 1,000 distinct antibodies per a surface area of less than about 1 cm². For example, an array can comprise 50, 60, 70, 80, 90, 100, 150, 200, 250, 300, 350 or 400 discrete regions in an area of about 16 mm², or 2,500 discrete regions/cm².

### IV. Peptide Agents

In some embodiments, the active agents are not AGEDPHGYFLPGQFA (SEQ ID NO: 38), AGEDPHSFYFPGQFA (SEQ ID NO: 1), AGEDPHSFYFPGQFAF, or AGEDPHSFYFPGQFAFS. In some embodiments, the active agents are analogs of C15 with sequences from 10-30 amino acids in length and not including the region from the human sequence PHSFYFPGQFA (SEQ ID NO: 41) analogous or homologous sequences.

In some embodiments, the active agents are analogs of C15, comprise chimeric sequences from 10-30 amino acids in length, and do not include the sequence AGEDPHGFYFPGQFA.

In some embodiments, the active agents are analogs of C15 with chimeric sequences including combinations of human chemerin sequences combined with sequences from other species. In certain embodiments, the middle amino acids of the sequence show the most diversity across species and are good positions to use to make the chimeric sequence.

In some embodiments, the active agents are not analogs of C15, comprise sequences from 10-30 amino acids in length, and do not include the region from the human sequence PHSFYFPGQFA (SEQ ID NO: 41), analogous or homologous sequences.

In some embodiments, the active agents are not analogs of C15, comprise chimeric sequences of 10-30 amino acids in length, and do not include the sequence AGEDPHGFYFPGQFA.

In some embodiments, the active agents are sequences of 10-30 amino acids in length containing natural amino acid substitutions (X) include but are not limited to:

XGEDPHSFYFPGQFA,
AXEDPHSFYFPGQFA,
AGXDPHSFYFPGQFA,
AGEXPHSFYFPGQFA,
AGEDPXSFYFPGQFA,
AGEDPXSFYFPGQFA,
AGEDPHXFYFPGQFA,
AGEDPHSXYFPGQFA,
AGEDPHSFXFPGQFA,
AGEDPHSFYXPGQFA,
AGEDPHSFYFXGQFA,
AGEDPHSFYFPXQFA,
AGEDPHSFYFPGXFA,
AGEDPHSFYFPGQXA,
AGEDPHSFYFPGQFX,
AGEDPHSXYX′PGQFA,
and
AGEDPHSXX′X″PGQFA.

In some embodiments, X, X′ and X″ are the same or different.

In some embodiments, the natural amino acids are selected from commercially available naturally occurring amino acids of the D- or L-configuration.

In some embodiments, the active agents are sequences of 10-30 amino acids in length containing unnatural amino acids (U) include but are not limited to:

UGEDPHSFYFPGQFA,
AUEDPHSFYFPGQFA,
AGUDPHSFYFPGQFA,
AGEUPHSFYFPGQFA,
AGEDUHSFYFPGQFA,
AGEDPUSFYFPGQFA,
AGEDPHUFYFPGQFA,
AGEDPHSUYFPGQFA,
AGEDPHSFUFPGQFA,
AGEDPHSFYUPGQFA,
AGEDPHSFYFUGQFA,
AGEDPHSFYFPUQFA,
AGEDPHSFYFPGUFA,
AGEDPHSFYFPGQUA,
AGEDPHSFYFPGQFU,
AGEDPHSUYU′PGQFA,
and
AGEDPHSUU″U′PGQFA.

In some embodiments a U is p-chloro phenyl anm. In some embodiments a U is homo-serine. In some embodiments, the unnatural amino acids are each independently commercially available amino acids of the D-configuration, L-configuration, or achiral amino acids which do not occur in nature (e.g. listed in the Accelrys Available Chemicals Directory (ACD)) and selected for improvements to the antagonist sequences solubility, stability, potency, mechanism of action, pharmaceutical properties.

In some embodiments, the active agents are natural and chimeric sequences containing up to 50% unnatural amino acids.

In some embodiments, the active agents are hybrid peptides and nonpeptides containing peptide sequences known to enhance the penetration of peptide or peptide/nonpeptide hybrids into the cell. This includes sequences such as but not limited to:

RRRRRRRAGEDPHSFYFPGQFA,
and
RRRRRRR-[NonPeptideSM],

where the -[NonPeptideSM] is a small molecule non-peptide antagonist of C15 binding to PP2A, small molecule non-peptide antagonist of PR70 or PR72 binding to PP2A, and/or antagonist of the NF-κB Classical Pathway.

In further or additional embodiments, the active agent has the sequence: a f q g p f y f s h p d e g a (where lower case amino acid single letter code denotes the D-configuration. This sequence would be known as the retro-inverso peptide sequence). In some embodiments, further retro-inverso sequences representing chemerin C-terminal fragments of non-human chemerin sequences, chimeric sequences and retro inverso sequences containing unnatural amino acids are selected from commercially available unnatural amino acids (e.g. listed in the Accelrys Available Chemicals Directory (ACD), http://accelrys.com) and selected for improvements to the antagonist sequences solubility, stability, potency, mechanism of action, pharmaceutical properties.

### V. Diseases and Disorders

Disclosed herein, in certain embodiments, are non-chemerin C15 peptides. Also disclosed herein are topical formulations comprising an agent that is not a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising an agent that is not a chemerin C15 peptide disclosed herein. Further disclosed herein are methods of inhibiting the activity of an inflammatory cytokine or chemokine in an individual in need thereof comprising administering an agent that is not a chemerin C15 peptide or a topical formulation comprising a chemerin C15 peptide. Also disclosed herein, in certain embodiments, are method of inhibiting inhibits nuclear translocation or NF-κB-mediated gene transcription of an inflammatory cytokine in an individual in need thereof comprising administering an agent that is not a chemerin C15 peptide or a topical formulation comprising a chemerin C15 peptide. In some embodiments, the agent that is not a chemerin C15 peptide is a salt of a non-chemerin C15 peptide. In some embodiments, the agent that is not a chemerin C15 peptide is carboxylated. In some embodiments, the agent that is not a chemerin C15 peptide is amidated. In some embodiments, the agent that is not a chemerin C15 peptide is cyclic.

Some embodiments provided herein describe methods of treating a disease or disorder in a subject, the method comprising administration to the subject a composition comprising an active agent described herein. In some embodiments, the agent is a peptide. In some embodiments, the agent is a small molecule. In some embodiments, the agent is a nucleic acid. In some embodiments, the agent is an antibody.

In some embodiments, the disease or disorder is an inflammatory disease. Non-limiting examples of inflammatory disorders include Psoriasis, Atopic Dermatitis, Contact Dermatitis, Lichen Planus, Acne, and Alopecia Areata, IBD, Crohn's Disease and/or Ulcerative Colitis, Uveitis, Dry Eye, Blepharitis, Allergic conjunctivitis, Iritis, retinal inflammatory diseases including AMD, and DME. Additional inflammatory diseases of interest include: Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barre syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behcet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease; abdominal aortic aneurysm (AAA); or combinations thereof.

In some embodiments, the methods and compositions described herein treat inflammation (e.g., acute or chronic). In certain instances, inflammation results from (either partially or fully) an infection. In certain instances, inflammation results from (either partially or fully) damage to a tissue (e.g., by a burn, by frostbite, by exposure to a cytotoxic agent, or by trauma). In certain instances, inflammation results from (either partially or fully) an autoimmune disorder. In certain instances, inflammation results from (either partially or fully) the presence of a foreign body (e.g., a splinter). In certain instances, inflammation results from exposure to a toxin and/or chemical irritant.

Multiple disorders are associated with inflammation (i.e., inflammatory disorders). Inflammatory disorders include, but are not limited to, Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barre syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behcet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rej ection, Chronic transplant rejection, Acute graft-versus-host disease, Chronic graft-versus-host disease, or combinations thereof.

In some embodiments, the disease or disorder is multiple sclerosis, sepsis, myasthenia gravis, autoimmune neuropathies, Guillain-Barre syndrome, autoimmune uveitis, autoimmune hemolytic anemia, pernicious anemia, autoimmune thrombocytopenia, temporal arteritis, anti-phospholipid syndrome, vasculitides, Wegener's granulomatosis, Behcet's disease, psoriasis, psoriatic arthritis, dermatitis herpetiformis, pemphigus vulgaris, vitiligo, Crohn's disease, ulcerative colitis, interstitial pulmonary fibrosis, myelofibrosis, hepatic fibrosis, myocarditis, thyroditis, primary biliary cirrhosis, autoimmune hepatitis, immune-mediated diabetes mellitus, Grave's disease, Hashimoto's thyroiditis, autoimmune oophoritis and orchitis, autoimmune disease of the adrenal gland, rheumatoid arthritis, juvenile rheumatoid arthritis, systemic lupus erythematosus, scleroderma, common variable immunodeficiency (CVID), polymyositis, dermatomyositis, spondyloarthropathies, ankylosing spondylitis, Sjögren's syndrome or graft-versus-host disease. In some embodiments, the disease or disorder is psorasis.

In some embodiments, the methods and compositions described herein treat an acute inflammatory disorder. As used herein, “acute inflammation” refers to inflammation characterized in that it develops over the course of a few minutes to a few hours, and ceases once the stimulus has been removed (e.g., an infectious agent has been killed by an immune response or administration of a therapeutic agent, a foreign body has been removed by an immune response or extraction, or damaged tissue has healed). The short duration of acute inflammation results from the short half-lives of most inflammatory mediators. In some embodiments, the acute inflammatory disorders is graft versus host disease, transplant rejection, septic shock, endotoxemia, Lyme arthritis, infectious meningitis {e.g., viral, bacterial, Lyme disease-associated), an acute episode of asthma or acute episodes of an autoimmune disease.

In certain instances, acute inflammation begins with the activation of leukocytes (e.g., antigen presenting cells, dendritic cells, neutrophils and mastocytes). In certain instances, the leukocytes release inflammatory mediators (e.g., histamines, proteoglycans, serine proteases, eicosanoids, and cytokines). In certain instances, inflammatory mediators result in (either partially or fully) the symptoms associated with inflammation. For example, in certain instances an inflammatory mediator dilates post capillary venules, and increases blood vessel permeability. In certain instances, the increased blood flow that follows vasodilation results in (either partially or fully) rubor and calor. In certain instances, increased permeability of the blood vessels results in an exudation of plasma into the tissue leading to edema. In certain instances, the latter allows leukocytes to migrate along a chemotactic gradient to the site of the inflammatory stimulant. Further, in certain instances, structural changes to blood vessels (e.g., capillaries and venules) occur. In certain instances, the structural changes are induced (either partially or fully) by monocytes and/or macrophages. In certain instances, the structural changes include, but are not limited to, remodeling of vessels, and angiogenesis. In certain instances, angiogenesis contributes to the maintenance of chronic inflammation by allowing for increased transport of leukocytes.

Additionally, in certain instances, histamines and bradykinin irritate nerve endings leading to itching and/or pain.

In some embodiments, the methods and compositions described herein treat a chronic inflammatory disorder. In certain instances, chronic inflammation results from the presence of a persistent stimulant (e.g., persistent acute inflammation, bacterial infection (e.g., by Mycobacterium tuberculosis), prolonged exposure to chemical agents (e.g., silica, or tobacco smoke) and autoimmune reactions (e.g., rheumatoid arthritis)). In certain instances, the persistent stimulant results in continuous inflammation (e.g., due to the continuous recruitment of monocytes, and the proliferation of macrophages). In certain instances, the continuous inflammation further damages tissues which results in the additional recruitment of mononuclear cells thus maintaining and exacerbating the inflammation. In certain instances, physiological responses to inflammation further include angiogenesis and fibrosis. In some embodiments, the chronic inflammatory disorder is asthma, rubella arthritis, and chronic autoimmune diseases, such as systemic lupus erythematosus, psoriasis, inflammatory bowel disease, including Crohn's disease and ulcerative colitis, multiple sclerosis or rheumatoid arthritis.

In some embodiments, the methods and compositions described herein treat an immune disease or disorder. Examples of immune-related diseases or disorders include, without limitation, rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/Wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, diabetes, nephrosis, atopic diseases, hypersensitity reactions, allergic rhinitis, hay fever, perennial rhinitis, conjunctivitis, endometriosis, asthma, urticaria, systemic anaphalaxis, dermatitis, pernicious anemia, hemolytic disesease, thrombocytopenia, graft rejection of any organ or tissue, kidney transplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, antibody-meditated cytotoxicity, type in hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetic retinopathy, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited toasthenia, anemia, cachexia, and the like), chronic salicylate intoxication, and the like.

In some embodiments, the methods and compositions described herein treat a T-cell mediated autoimmune disorder. In certain instances, a T-cell mediated autoimmune disorder is characterized by a T-cell mediated immune response against self (e.g., native cells and tissues).

Examples of T-cell mediated autoimmune disorders include, but are not limited to colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjogren's syndrome, systemic lupus erythematosus (SLE), vasculitis syndromes (e.g., giant-cell arteritis, Behcet's disease & Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes and Gullain-Barre disease.

In some embodiments, the methods and compositions described herein treat pain. Pain includes, but is not limited to acute pain, acute inflammatory pain, chronic inflammatory pain and neuropathic pain.

In some embodiments, the methods and compositions described herein treat hypersensitivity. As used herein, “hypersensitivity” refers to an undesirable immune system response. Hypersensitivity is divided into four categories. Type I hypersensitivity includes allergies (e.g., Atopy, Anaphylaxis, or Asthma). Type II hypersensitivity is cytotoxic/antibody mediated (e.g., Autoimmune hemolytic anemia, Thrombocytopenia, Erythroblastosis fetalis, or Goodpasture's syndrome). Type III is immune complex diseases (e.g., Serum sickness, Arthus reaction, or SLE). Type IV is delayed-type hypersensitivity (DTH), Cell-mediated immune memory response, and antibody-independent (e.g., Contact dermatitis, Tuberculin skin test, or Chronic transplant rejection).

In some embodiments, the methods and compositions described herein treat an allergy. As used herein, “allergy” means a disorder characterized by excessive activation of mast-cells and basophils by IgE. In certain instances, the excessive activation of mast-cells and basophils by IgE results (either partially or fully) in an inflammatory response. In certain instances, the inflammatory response is local. In certain instances, the inflammatory response results in the narrowing of airways (i.e., bronchoconstriction). In certain instances, the inflammatory response results in inflammation of the nose (i.e., rhinitis). In certain instances, the inflammatory response is systemic (i.e., anaphylaxis).

In some embodiments, the methods and compositions described herein treat angiogenesis. As used herein, “angiogenesis” refers to the formations of new blood vessels. In certain instances, angiogenesis occurs with chronic inflammation. In certain instances, angiogenesis is induced by monocytes and/or macrophages. In certain instances, angiogenesis drives the pathogenesis of psoriasis.

In some embodiments the present disclosure comprises a method of treating a neoplasia. In certain instances, a neoplastic cell induces an inflammatory response. In certain instances, part of the inflammatory response to a neoplastic cell is angiogenesis. In certain instances, angiogenesis facilitates the development of a neoplasia. In some embodiments, the neoplasia being treated by the methods described herein is: angiosarcoma, Ewing sarcoma, osteosarcoma, and other sarcomas, breast carcinoma, cecum carcinoma, colon carcinoma, lung carcinoma, ovarian carcinoma, pharyngeal carcinoma, rectosigmoid carcinoma, pancreatic carcinoma, renal carcinoma, endometrial carcinoma, gastric carcinoma, liver carcinoma, head and neck carcinoma, breast carcinoma and other carcinomas, Hodgkins lymphoma and other lymphomas, malignant and other melanomas, parotid tumor, chronic lymphocytic leukemia and other leukemias, astrocytomas, gliomas, hemangiomas, retinoblastoma, neuroblastoma, acoustic neuroma, neurofibroma, trachoma and pyogenic granulomas.

Some embodiments provided herein describe methods of treating cancer, wherein the method comprises treating a patient with any one of the active agents described herein. In some embodiments, the active agents suppress the expression of genes involved in carcinogenesis and tumor genesis. In some embodiments, the methods described herein treat cancers such as lung, breast, brain, prostate, spleen, pancreatic, cervical, ovarian, head and neck, esophageal, liver, skin, kidney, leukemia, bone, testicular, colon, or bladder cancer. In certain embodiments, the cancer is pancreatic cancer, colon cancer, breast cancer, T-cell leukemias, or lymphomas. In some embodiments, the cancer is leukemia, lymphoma, or multiple myeloma.

Examples of cancer include but are not limited to cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; gianT-cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; masT-cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; masT-cell leukemia; megakaryoblastic leukemia; myeloid sarcoma; and hairy cell leukemia.

In some embodiments, the methods and compositions described herein treat obesity. As used herein, “obesity” means an accumulation of adipose tissue with a BMI of greater than or equal to 30 kg/m². In certain instances, obesity is characterized a proinflammatory state, increasing the risk of thrombosis. In certain instances, obesity is associated with a low-grade inflammation of white adipose tissue (WAT). In certain instances, WAT associated with obesity is characterized by an increased production and secretion of a wide range of inflammatory molecules including TNF-alpha and interleukin-6 (IL-6). In certain instances, WAT is infiltrated by macrophages, which produce pro-inflammatory cytokines. In certain instances, TNFα is overproduced in adipose tissue. In certain instances, IL-6 production increases during obesity.

In some embodiments, the methods and compositions described herein treat a metabolic syndrome. In certain instances, the metabolic syndrome is associated with fasting hyperglycemia; high blood pressure; central obesity; decreased HDL levels; elevated triglyceride levels; systemic inflammation; or combinations thereof. In certain instances, metabolic syndrome is characterized by an increase in the levels of C-reactive protein, fibrinogen, (IL-6), and TNFα.

In some embodiments, the methods and compositions described herein treat an aneurysm. In certain instances, an atherosclerotic plaque results (partially or fully) in the development of an aneurysm. In some embodiments, the methods and compositions described herein treat an abdominal aortic aneurysm (“AAA”). As used herein, an “abdominal aortic aneurysm” is a localized dilatation of the abdominal aorta. In certain instances, the rupture of an AAA results in bleeding, leading to hypovolemic shock with hypotension, tachycardia, cyanosis, and altered mental status.

In some embodiments, the compositions and methods disclosed herein treat abdominal aortic aneurysms. In certain instances, abdominal aortic aneurysms result (partially or fully) from an extensive breakdown of structural proteins (e.g., elastin and collagen). In some embodiments, a method and/or composition disclosed herein partially or fully inhibits the breakdown of a structural protein (e.g., elastin and collagen). In certain instances, the breakdown of structural proteins is caused by activated MMPs. In some embodiments, a method and/or composition disclosed herein partially or fully inhibit the activation of an MMP. In some embodiments, a composition and/or method disclosed herein inhibit the upregulation of MMP-1, MMP-9 or MMP-12. In certain instances, MIF is co-expressed with MMP-1, MMP-9, and MMP-12 in abdominal aortic aneurysms. In certain instances, the MIF is upregulated in stable abdominal aortic aneurysm and is intensified further in ruptured aneurysms. In certain instances, MMPs are activated following infiltration of a section of the abdominal aorta by leukocytes (e.g., macrophages and neutrophils). In some embodiments, a method and/or composition disclosed herein partially or fully inhibits the activity of MIF. In some embodiments, a method and/or composition disclosed herein partially or fully inhibit the infiltration of a section of the abdominal aorta by leukocytes.

In some embodiments, the methods and compositions described herein treat a neurological disorder. In some embodiments, the neurological disease, disorder or condition is caused, exasperated, or mediated by NF-κB. In some embodiments, the neurological disease, disorder or condition is caused, exasperated, or mediated by IL-12, IL-23 and/or IL-27 production. Such neurological disorders include, without limitation, neurodegenerative diseases, multiple sclerosis, migraine headache, AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia telangiectasia, and mitochondrial multi-system disorder); demyelinating core disorders, such as multiple sclerosis, acute transverse myelitis; and disorders of the motor unit’ such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; and Dementia pugilistica, and the like.

In some embodiments, the methods and compositions described herein treat wounds. In some embodiments, the methods and compositions described herein enhance wound healing. In some embodiments, the methods and compositions described herein enhance would healing and reduce scarring.

### VI. Combinations

### Anti-Inflammatory Agents

The terms “anti-inflammatory agent” and “modulator of inflammation” are used interchangeably. As used herein, the terms refer to agents treat inflammation and/or an inflammatory disorder. In some embodiments, the anti-inflammatory agent is an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent (e.g., cyclosporine A), an alkylating agent, an anti-metabolite, a plant alkaloid, a terpenoids, a topoisomerase inhibitor, an antitumor antibiotic, an antibody, a hormonal therapy (e.g., aromatase inhibitors), a leukotriene inhibitor, or combinations thereof.

In some embodiments, an agent that binds to PP2A core enzyme in administered in combination with a second anti-inflammatory agent. In some embodiments, the second anti-inflammatory agent is: cyclosporine A, lifitegrast, alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, sulfasalazine, etanercept, adalimumab, infliximab, abatacept, rituximab, trastuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (NIAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (NIAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-lb, interferon beta-la, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-lbeta monoclonal antibody, Novartis), CNTO 1275 (Fully Human Anti-IL-12 Monoclonal Antibody, Centocor), (3 S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (anti IL-12 monoclonal antibody, Abbott Labs), MRA(Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol; Actos® (Pioglitazone), Avandia® (Rosiglitazone), Amaryl® (Glimepiride), Sulfonylurea-types, Diabeta® (Glyburide), Diabinese® (Chlorpropamide), Glucotrol® (Glipizide), Glynasec (glyburide), Micronase® (glyburide), Orinase® (Tolbutamide), Tolinase® (Tolazamide), Glucophage, Riomet® (Metformin), Glucovance® (glyburide+metformin), Avandamet® (Rosiglitazone+metformin), Avandaryl® (Rosiglitazone+glimepiride), Byetta® (Exenatide), Insulins, Januvia® (Sitagliptin), Metaglip® (glipizide and metformin), Prandin® (Repaglinide), Precose® (Acarbose), Starlix® (Nateglinide), Xenical® (Orlistat), cisplatin; carboplatin; oxaliplatin; mechlorethamine; cyclophosphamide; chlorambucil; vincristine; vinblastine; vinorelbine; vindesine; azathioprine; mercaptopurine; fludarabine; pentostatin; cladribine; 5-fluorouracil (5FU); floxuridine (FUDR); cytosine arabinoside; methotrexate; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; trastuzumab; cetuximab; rituximab; bevacizumab; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl) indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimehtylpropanoic acid); RP64966 ([4-[5-(3-Phenyl-propyl)thiophen-2-yl]butoxy]acetic acid); SA6541 ((R)—S-[[4-(dimethylamino)phenyl]methyl]-N-(3-mercapto-2methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(H)-quinlolinone); busulphan; alemtuzumab; belatacept (LEA29Y); posaconazole; fingolimod (FTY720); an anti-CD40 ligand antibody (e.g., BG 9588); CTLA4Ig (BMS 188667); abetimus (LJP 394); an anti-IL10 antibody; an anti-CD20 antibody (e.g. rituximab); an anti-C5 antibody (e.g., eculizumab); doxycycline; or combinations thereof.

In some embodiments, an agent that binds to PP2A core enzyme is in administered in combination with and a 5-aminosalicylic acid (5-ASA) to treat an inflammatory disorder. In some embodiments, a method comprises administering an therapeutically effective amount of agent that binds to PP2A core enzyme and a 5-aminosalicylic acid (5-ASA) to a subject with an inflammatory disorder, thereby providing a therapeutically effective decrease in leukocyte chemotaxis and reduction in eicosanoid and inflammatory cytokine synthesis.

In some embodiments, an agent that binds to PP2A core enzyme is in administered in combination with an anti-TNF agent to treat an inflammatory disorder. In some embodiments, a method comprises administering a therapeutically effective amount of agent that binds to PP2A core enzyme and an anti-TNF agent treat to a subject with an inflammatory disorder, thereby providing a therapeutically effective decrease in leukocyte chemotaxis and suppression in TNF-induced cytokine activity.

In some embodiments, an agent that binds to PP2A core enzyme is in administered in combination with a leukotriene inhibitor to treat an inflammatory disorder. In some embodiments, a method comprises administering a therapeutically effective amount of agent that binds to PP2A core enzyme and a leukotriene inhibitor to a subject with an inflammatory disorder, thereby providing a therapeutically effective (1) decrease in leukocyte chemotaxis; and (2) antagonization of LTA4, LTB4, LTC4, LTD4, LTE4, LTF4, LTA4R; LTB4R; LTB4R1, LTB4R2, LTC4R, LTD4R, LTE4R, CYSLTR1, or CYSLTR2; and/or inhibition of a leukotriene synthesis via 5-LO, FLAP, LTA4H, LTA4S, or LTC4S.

In some embodiments, the leukotriene inhibitor is montelukast, a methyl xanthine, zafirlukast, or zileuton.

In some embodiments, an agent that binds to PP2A core enzyme described herein is administered in combination with a beta2-agonist. In certain embodiments, the beta2-agonist is albuterol, biterol, fenoterol, isoetharie, metaproterenol, pirbuterol, salbutamol, terbutalin formoterol, salmeterol, or salbutamol terbutaline.

In some embodiments, an agent that binds to PP2A core enzyme described herein is administered in combination with a non-steroidal anti-inflammatory agent. In some embodiments, the anti-inflammatory agent is selected from the group consisting of aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone.

In some embodiments, an agent that binds to PP2A core enzyme is in administered in combination with a IL-1 receptor antagonist to treat an inflammatory disorder. In some embodiments, a method comprises administering a therapeutically effective amount of agent that binds to PP2A core enzyme and a IL-1 receptor antagonist to a subject with an inflammatory disorder, thereby providing a therapeutically effective (1) decrease in leukocyte chemotaxis; and (2) blocking of the stimulation of T-cell IL-1 receptor.

In some embodiments, an agent that binds to PP2A core enzyme is in administered in combination with a IL-2 receptor antagonist to treat an inflammatory disorder. In some embodiments, a method comprises administering a therapeutically effective amount of agent that binds to PP2A core enzyme and a IL-2 receptor antagonist to a subject with an inflammatory disorder, thereby providing a therapeutically effective (1) decrease in leukocyte chemotaxis; and (2) blocking of the stimulation of T-cell IL-2 receptor.

In some embodiments, an agent that binds to PP2A core enzyme is in administered in combination with a cytotoxic agent to treat an inflammatory disorder. In some embodiments, a method comprises administering a therapeutically effective amount of agent that binds to PP2A core enzyme and a cytotoxic agent to a subject with an inflammatory disorder, thereby providing a therapeutically effective (1) decrease in leukocyte chemotaxis; and (2) treatment of a neoplastic disease.

In some embodiments, an agent that binds to PP2A core enzyme is in administered in combination with an immunomodulatory agent to treat an inflammatory disorder. In some embodiments, a method comprises administering a therapeutically effective amount of agent that binds to PP2A core enzyme and an immunomodulatory agent to a subject with an inflammatory disorder, thereby providing a therapeutically effective (1) decrease in leukocyte chemotaxis; and (2) enhancement or suppression of the immune system.

In some embodiments, an agent that binds to PP2A core enzyme is in administered in combination with an antibiotic to treat an inflammatory disorder. In some embodiments, a method comprises administering a therapeutically effective amount of agent that binds to PP2A core enzyme and an antibiotic to a subject with an inflammatory disorder, thereby providing a therapeutically effective (1) decrease in leukocyte chemotaxis; and (2) inhibition of cell and/or microbial growth, such as by disrupting the cell cycle (e.g., by disrupting histone deacetylase). In some embodiments, the antibiotic is dactinomycin (formerly actinomycin), bleomycin, erythomycin, penicillin, mithramycin, or anthramycin (AMC).

In some embodiments, an agent that binds to PP2A core enzyme is in administered in combination with T-cell co-stimulatory blocker to treat an inflammatory disorder. In some embodiments, a method comprises administering a therapeutically effective amount of agent that binds to PP2A core enzyme and T-cell co-stimulatory blocker to a subject with an inflammatory disorder, thereby providing a therapeutically effective (1) decrease in leukocyte chemotaxis; and (2) modulation of a co-stimulatory signal which is required for full T-cell activation.

In some embodiments, an agent that binds to PP2A core enzyme is in administered in combination with a B cell depleting agent to treat an inflammatory disorder. In some embodiments, a method comprises administering a therapeutically effective amount of agent that binds to PP2A core enzyme and a B cell depleting agent to a subject with an inflammatory disorder, thereby providing a therapeutically effective (1) decrease in leukocyte chemotaxis; and (2) inhibition of B-cell activity.

In some embodiments, an agent that binds to PP2A core enzyme is in administered in combination with an immunosuppressive agent to treat an inflammatory disorder. In some embodiments, a method comprises administering a therapeutically effective amount of agent that binds to PP2A core enzyme and an immunosuppressive agent to a subject with an inflammatory disorder, thereby providing a therapeutically effective (1) decrease in leukocyte chemotaxis; and (2) selective or non-selective inhibition or prevention of activity of the immune system.

In some embodiments, an agent that binds to PP2A core enzyme is in administered in combination with an alkylating agent to treat an inflammatory disorder. In some embodiments, a method comprises administering a therapeutically effective amount of agent that binds to PP2A core enzyme and an alkylating agent to a subject with an inflammatory disorder, thereby providing a therapeutically effective (1) decrease in leukocyte chemotaxis; and (2) induction of covalent binding of alkyl groups to cellular molecules.

In some embodiments, an agent that binds to PP2A core enzyme is in administered in combination with an anti-metabolite to treat an inflammatory disorder. In some embodiments, a method comprises administering a therapeutically effective amount of agent that binds to PP2A core enzyme and an anti-metabolite to a subject with an inflammatory disorder, thereby providing a therapeutically effective (1) decrease in leukocyte chemotaxis; and (2) prevention of biosynthesis or use of normal cellular metabolites.

In some embodiments, an agent that binds to PP2A core enzyme is in administered in combination with plant alkaloid to treat an inflammatory disorder. In some embodiments, a method comprises administering a therapeutically effective amount of agent that binds to PP2A core enzyme and plant alkaloid to a subject with an inflammatory disorder, thereby providing a therapeutically effective (1) decrease in leukocyte chemotaxis; and (2) interference with normal microtubule breakdown during cell division.

In some embodiments, an agent that binds to PP2A core enzyme is in administered in combination with a terpenoid to treat an inflammatory disorder. In some embodiments, a method comprises administering a therapeutically effective amount of agent that binds to PP2A core enzyme and a terpenoid to a subject with an inflammatory disorder, thereby providing a therapeutically effective (1) decrease in leukocyte chemotaxis; and (2) treatment of neoplastic disease or microbial infections.

In some embodiments, an agent that binds to PP2A core enzyme is in administered in combination with a topoisomerase inhibitor to treat an inflammatory disorder. In some embodiments, a method comprises administering a therapeutically effective amount of agent that binds to PP2A core enzyme and a topoisomerase inhibitor to a subject with an inflammatory disorder, thereby providing a therapeutically effective (1) decrease in leukocyte chemotaxis; and (2) modulation of cellular topoisomerase enzyme activity.

In some embodiments, an agent that binds to PP2A core enzyme is in administered in combination with an antibody to treat an inflammatory disorder. In some embodiments, a method comprises administering a therapeutically effective amount of agent that binds to PP2A core enzyme and an antibody to a subject with an inflammatory disorder, thereby providing a therapeutically effective (1) decrease in leukocyte chemotaxis; and (2) neutralization of inflammatory cytokines, such as, for example, TNF alpha.

In some embodiments, an agent that binds to PP2A core enzyme is in administered in combination with a hormonal therapy to treat an inflammatory disorder. In some embodiments, a method comprises administering a therapeutically effective amount of agent that binds to PP2A core enzyme and a hormonal therapy to a subject with an inflammatory disorder, thereby providing a therapeutically effective (1) decrease in leukocyte chemotaxis; and (2) suppression of cytokine release.

In some embodiments, an agent that binds to PP2A core enzyme is in administered in combination with an anti-diabetes therapy to treat an inflammatory disorder. In some embodiments, a method comprises administering a therapeutically effective amount of agent that binds to PP2A core enzyme and an anti-diabetes therapy to a subject with an inflammatory disorder, thereby providing a therapeutically effective (1) decrease in leukocyte chemotaxis; and (2) improvement in sensitivity to insulin in muscle and adipose tissue.

### Anti-Cancer Agents

In some embodiments, an active agent described herein is administered in combination with a chemotherapeutic agent. In some embodiments, the chemotherapeutic agent is cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, gemcitabien, navelbine, famesyl-protein tansferase inhibitors, transplatinum, 5-fluorouracil, vincristin, Velcade, vinblastin, methotrexate, or any analog or derivative variant of the foregoing.

In some embodiments, an active agent described herein is administered in combination with radiotherapy. Non-limiting examples of radio therapy include γ-rays, X-rays, and/or the directed delivery of radioisotopes to tumor cells. In certain instances, microwaves and/or UV-irradiation are used according to methods of the disclosure. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### VII. Pharmaceutical Compositions

Disclosed herein, in certain embodiments, is a pharmaceutical composition for modulating an inflammation, comprising a synergistic combination of (a) a therapeutically-effective amount of an agent that binds to PP2A core enzyme and; and (b) a therapeutically-effective amount of a second active agent.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, the pharmaceutical composition for modulating an inflammation further comprises a pharmaceutically acceptable diluent(s), excipient(s), or carrier(s). In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions described herein are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, modified release formulations, delayed release formulations, extended release formulations, pulsatile release formulations, multiparticulate formulations, and mixed immediate release and controlled release formulations.

Pharmaceutically acceptable salts of the agents of this disclosure include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include alkali metal (e.g., sodium), alkaline earth metal (e.g., magnesium), ammonium and N-(alkyl)₄⁺ salts. This disclosure also envisions the quaternization of any basic nitrogen-containing groups of the agents disclosed herein.

Water or oil-soluble or dispersible products may be obtained by such quaternization

### Multiparticulate Formulations

In some embodiments, the pharmaceutical compositions described herein are formulated as mulitparticulate formulations. In some embodiments, the pharmaceutical compositions described herein comprise a first population of particles and a second population of particles. In some embodiments, the first population comprises an active agent. In some embodiments, the second population comprises an active agent. In some embodiments, the dose of active agent in the first population is equal to the dose of active agent in the second population. In some embodiments, the dose of active agent in the first population is not equal to (e.g., greater than or less than) the dose of active agent in the second population.

In some embodiments, the active agent of the first population is released before the active agent of the second population. In some embodiments, the second population of particles comprises a modified-release (e.g., delayed-release, controlled-release, or extended release) coating. In some embodiments, the second population of particles comprises a modified-release (e.g., delayed-release, controlled-release, or extended release) matrix.

Coating materials for use with the pharmaceutical compositions described herein include, but are not limited to, polymer coating materials (e.g., cellulose acetate phthalate, cellulose acetate trimaletate, hydroxy propyl methylcellulose phthalate, polyvinyl acetate phthalate); ammonio methacrylate copolymers (e.g., Eudragit® RS and RL); poly acrylic acid and poly acrylate and methacrylate copolymers (e.g., Eudragite S and L, polyvinyl acetaldiethylamino acetate, hydroxypropyl methylcellulose acetate succinate, shellac); hydrogels and gel-forming materials (e.g., carboxyvinyl polymers, sodium alginate, sodium carmellose, calcium carmellose, sodium carboxymethyl starch, poly vinyl alcohol, hydroxyethyl cellulose, methyl cellulose, gelatin, starch, hydoxypropyl cellulose, hydroxypropyl methylcellulose, polyvinylpyrrolidone, crosslinked starch, microcrystalline cellulose, chitin, aminoacryl-methacrylate copolymer, pullulan, collagen, casein, agar, gum arabic, sodium carboxymethyl cellulose, (swellable hydrophilic polymers) poly(hydroxyalkyl methacrylate) (MW about 5 k to about 5,000 k), polyvinylpyrrolidone (MW about 10 k to about 360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (MW about 30 k to about 300 k), polysaccharides such as agar, acacia, karaya, tragacanth, algins and guar, polyacrylamides, Polyox® polyethylene oxides (MW about 100 k to about 5,000 k), AquaKeep® acrylate polymers, diesters of polyglucan, crosslinked polyvinyl alcohol and poly N-vinyl-2-pyrrolidone, sodium starch; hydrophilic polymers (e.g., polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid, other acrylic acid derivatives, sorbitan esters, natural gums, lecithins, pectin, alginates, ammonia alginate, sodium, calcium, potassium alginates, propylene glycol alginate, agar, arabic gum, karaya gum, locust bean gum, tragacanth gum, carrageens gum, guar gum, xanthan gum, scleroglucan gum); or combinations thereof. In some embodiments, the coating comprises a plasticiser, a lubricant, a solvent, or combinations thereof. Suitable plasticisers include, but are not limited to, acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

In some embodiments, the second population of particles comprises a modified release matrix material. Materials for use with the pharmaceutical compositions described herein include, but are not limited to microcrystalline cellulose, sodium carboxymethylcellulose, hydoxyalkylcelluloses (e.g., hydroxypropylmethylcellulose and hydroxypropylcellulose), polyethylene oxide, alkylcelluloses (e.g., methylcellulose and ethylcellulose), polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate, or combinations thereof.

### Other Formulations

Suitable formulations can be found in U.S. Pub. No. US20100150990 and International Pub. No. WO2013/056147, incorporated herein by reference in their entirety. Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active agent doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations disclosed herein are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a MIF receptor inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a MIF receptor inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are elf-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the pharmaceutical compositions disclosed herein are optionally in a form of an aerosol, a mist or a powder. Pharmaceutical compositions described herein are conveniently delivered in the form of an aerosol spray presentation from pressurized packs or an Nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix and a suitable powder base such as lactose or starch.

Buccal formulations include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of the pharmaceutical compositions disclosed herein are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) an active agent; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In some embodiments, the transdermal formulations described herein are nonaqueous combinations of an active agent, penentration enhancer and a pharmaceutically acceptable excipient. In other embodiments, the transdermal formulations described herein maintain a saturated or supersaturated state to promote diffusion into the skin.

In some embodiments, formulations suitable for transdermal administration employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery is optionally accomplished by means of iontophoretic patches and the like. Additionally, transdermal patches provide controlled delivery. The rate of absorption is optionally slowed by using rate-controlling membranes or by trapping an active agent within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing an active agent optionally with carriers, optionally a rate controlling barrier to deliver a an active agent to the skin of the host at a controlled and predetermined rate over a prolonged period of time, and means to secure the device to the skin.

Formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene-glycol, polyethylene-glycol, glycerol, cremophor and the like), suitable mixtures thereof, vegetable oils (such as olive oil) and injectable organic esters such as ethyl oleate.

Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In some embodiments, an active agent disclosed herein is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives. In some embodiments, a topical composition disclosed herein comprises a penetration enhancer. In some embodiments, the penetration enhancer is isopropyl-myristate.

An active agent disclosed herein is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### VIII. Dosages and Administration

In some embodiments, the pharmaceutical compositions disclosed herein are administered to an individual in need thereof. In some embodiments, the pharmaceutical compositions disclosed herein are administered to an individual diagnosed with (i.e., satisfies the diagnostic criteria for) an inflammatory disorder (e.g. rheumatoid arthritis, SLE or cancer). In some embodiments, the pharmaceutical compositions disclosed herein are administered to an individual suspected of having an inflammatory disorder. In some embodiments, the pharmaceutical compositions disclosed herein are administered to an individual predisposed to develop an inflammatory disorder.

In certain instances, an individual is at risk of inflammatory bowel disease if elevated levels of bacterial antigens 12, OmpC or flagellin are present in the serum. In certain instances, an individual is at risk of Crohn's disease if perinuclear antineutrophil cytoplasmic antigens are detected in the serum. In certain instances, an individual is at risk of rheumatoid arthritis if the expression of IL-1β and its type II receptor is significantly upregulated in the blood. In certain instances, an individual is at risk of rheumatoid arthritis if the IL-6 levels are elevated in blood. In certain instances, an individual is at risk of SLE if MicroRNA 95 (miR 95) expression is one third of the gene expression of the microRNA 95 of controls. In certain instances, an individual is at risk of B-cell lymphoma if CD40 expression is upregulated on B cells. In certain instances, an individual is at risk of prostate cancer if PSA levels are elevated in blood.

The daily dosages appropriate for an active agent disclosed herein are from about 0.01 to 3 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages are optionally altered depending on a Number of variables, not limited to the activity of the active agents used, the diseases or conditions to be treated, the mode of administration, the requirements of the individual subject, the severity of the disease or condition being treated, and the judgment of the practitioner.

In some embodiments, administration of the lipid modulating agent results in (either partially or fully) undesired inflammation. In some embodiments, administration of the second anti-inflammatory agent results in (either partially or fully) undesired inflammation. In some embodiments, the first anti-inflammatory agent is administered to the individual to treat the undesired inflammation from the second anti-inflammatory agent or the lipid modulating agent. In some embodiments, the administration of the second anti-inflammatory agent or lipid modulating agent is discontinued until the inflamed cells and/or tissue are no longer inflamed. In some embodiments, after the inflamed cells and/or tissue are no longer inflamed, administration of the second inflammatory agent or lipid modulating agent recommences. In some embodiments, administration of the second anti-inflammatory agent or lipid modulating agent recommences in combination with an alternative dose of the first anti-inflammatory agent.

In the case wherein the individual's condition does not improve, upon the doctor's discretion the administration of an active agent disclosed herein is optionally administered chronically, that is, for an extended period of time, including throughout the duration of the individual's life in order to ameliorate or otherwise control or limit the symptoms of the individual's disease or condition.

In the case wherein the individual's status does improve, upon the doctor's discretion the administration of an active agent disclosed herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is therapeutic index, which is expressed as the ratio between LD₅₀ and ED₅₀. An active agent disclosed herein exhibiting high therapeutic indices is preferred. The data obtained from cell culture assays and animal studies are optionally used in formulating a range of dosage for use in human. The dosage of such an active agent disclosed herein lies preferably within a range of circulating concentrations that include the ED₅₀ with minimal toxicity. The dosage optionally varies within this range depending upon the dosage form employed and the route of administration utilized.

In some embodiments, methods disclosed herein are used before, during, and/or after an organ transplant. In some embodiments, compositions disclosed herein are administered before, during, and/or after an organ transplant. In certain instances, an “inflammatory/cytokine storm” develops following an organ transplant. In some embodiments, the organ transplant is a heart, kidney, lung transplant. In certain instances, an inflammatory cytokine storm comprises high fever, swelling and redness, extreme fatigue, and nausea. In some embodiments, an agent that binds to PP2A core enzyme and is administered in combination with cyclosporin A.

### Examples

### Example 1: Cell Study for PP2A/NF-κB Complex

Objective of Study:

To screen 1 test item at 3 dilutions in the human primary monocyte derived dendritic cell assay.

Experimental System:

Dendritic cells are matured from Human monocytes from 3 donors using the PromoCell system (C-28050). Dendritic cells are analyzed by flow cytometry to determine the percentage purity prior to use in the assay and are tested for ChemR23 expression.

The dendritic cells are treated with test agent at 3 concentrations for 1 hour prior to incubation with a stimulant (TBC) or culture medium (No stimulant). A vehicle and one reference article is included. All samples are run in triplicate. Cultures are stimulated for 0, 0.5 hr, 1 hr, 3 hrs, 15 hrs, 24 hrs and 48 hrs and cell culture supernatants are collected and assayed for inflammatory mediators using a Luminex bead-based assay.

Quantigene is used to measure mRNA in the cellular fraction for the following cytokines: Il-23p19, Il-12p35. NF-κB pathway markers are measured by ELISA for total cellular and phosphorylated NF-κB.

Cell viability is assessed with alamarBlue.

### Example 2—Phosphatase Assay

Purified GST-Cdc6 is phosphorylated in vitro by cyclin A/CDK2 (1/20 w/w) with 10 mM MgCl₂ and 10×molar concentration of ATP for 1 hr at 30° C. The phosphorylated protein is purified by gel filtration chromatography and then the GST-tag is cleaved with TEV protease. The pCdc6 peptide is then separated from GST or uncleaved peptide using ultrafiltration membrane with a 10 kDa cut-off.

The phosphatase activity of 50 nM-1 μM of PP2A core enzyme or holoenzyme containing wild type and mutant PR70 constructs are measured using 60 NVM pCdc6 peptide in a buffer containing 25 mM Tris pH 8.0, 150 mM NaCl, 3 mM DTT, 50 μM MnCl₂, and 1 mM CaCl₂. The reaction is performed in 50 μl assay volume at room temperature for 15 min and stopped by the addition of malachite green (100 μL). The absorbance at 620 nm is measured after 10 min incubation at room temperature. For steady state kinetics, the assays are performed using known concentrations of PP2A complexes and titration of pCdc6 peptide (10-1000 μM) in the presence or absence of small molecule agents #1-10, peptide agents #11-20, nucleic acid agents #21-30, and antibody agents #31-40. The data are fitted using GraphPad Prism (GraphPad Software Inc.) to calculate K_{M} and K_{cat}. The phosphatase activity of PP2A samples (0.1-100 μM) toward a universal phosphopeptide substrate (K-R-pT-I-R-R) is also measured. For steady state kinetics, the assays are performed using known concentrations of PP2A complexes and titration of pThr peptide (10 μM-100 mM) in the presence or absence of various test agents. All experiments are performed in triplicate and are repeated three times. Mean±SEM are calculated.

### Example 3—NF-κB Activation Assay

This example shows the inhibitory effects of various test agents on NF-κB activation. Treatment of the nuclear extract with the p65 antibody results in a significant decrease in NF-κB/DNA binding. As a negative control, cells are left inactivated and nuclear extracts are treated with the NF-κB consensus sequence, illustrating only a slight background level of NT-κB. Treatment of cells with camptothecin concentrations ranging from 10 μM to 10 nM illustrates a significant amount of NF-κB/DNA binding due to NF-κB activation.

Inhibition of camptothecin mediated activation of NF-κB by the agents disclosed herein is as follows: Induction of NF-κB activation can proceed via a wide range of signaling pathways (Delhase et al., Science 284: 309-313 (1999); Karin, Oncogene 18: 6867-6874 (1999)). Inhibition of NF-κB activation can proceed via the inhibition of many different pathways (Epinat and Gilmore, Oncogene 18: 6896-6909 (1999)). Modulators of these pathways may be therefore act as general activation inhibitors, whereas others may inhibit specific induction pathways (Epinat and Gilmore, Oncogene 18: 6896-6909 (1999)). To investigate whether the agents disclosed herein inhibit the specific pathway of camptothecin induced NF-κB activation, the inhibition of camptothecin induced NF-κB nuclear binding in the presence of the agents disclosed herein are examined.

Cells are treated with various concentration of small molecule agents #1-10, peptide agents #11-20, nucleic acid agents #21-30, and antibody agents #31-40, two minutes prior to activation by camptothecin (0.1 μM). The addition of small molecule agent #2 may inhibit camptothecin induced NF-κB nuclear binding in a dose responds manner. Cells are treated with various concentration of small molecule agents #1-10, peptide agents #11-20, nucleic acid agents #21-30, and antibody agents #31-40, two minutes, five minutes, ten minutes, twenty minutes, thirty minutes, one hour, 2 hours, 6 hours, 8 hours, 16 hours, and 24 hours, and so forth after to activation by camptothecin (0.1 μM) as well.

Antibody agents and small molecule agents and peptide agents and other agents will be evaluated for their ability to enhance the activity of camptothecin in CEM cells. Induction of apoptosis is the hallmark of most chemotherapeutic agents including camptothecin. The systematic disassembly of apoptotic cells is accomplished by active caspases (Thornberry et al., Nature 356: 768-774 (1992); Nicholson et al., Trends Biochem. Sci. 22: 299-306 (1997)). To test whether NF-κB inhibition enhances the activity of chemotherapeutic agents via the inhibition of anti-apoptotic signaling pathway, the effects of the agents are tested using a caspase-3/7 assay (Promega), which takes advantage of this caspase activity to directly quantify the induction of apoptosis in cells (Thornberry et al., Nature 356: 768-774 (1992); Nicholson et al., Trends Biochem. Sci. 22: 299-306 (1997)). This assay can quantify the level of apoptotic cell death induced by camptothecin with and without the agents and may establish the direct level of enhancement of apoptosis by camptothecin.

The antibody agent #32 appears non-toxic (or at least exhibit no significant cytotoxic effects in the cells). Antibody agent #32 may appear to significantly induce apoptosis when the agents are used in combination with the topoisomerase inhibitor, camptothecin (CPT). The concentration of camptothecin is kept constant at 0.1 μM in all experiments and a significant, dose-time response induction of apoptosis may be noted upon combinational treatment with the agents.

No significant induction of cell death may be observed when cells are treated with only the small molecule agents #9 and #10 up to 1.0 μM over 48 hours in this apoptosis (caspase-3) assay as and by cell count up to 10 μM over 72 hours.

This may be determined by the number of apoptotic cell death after 48 hours after treatment of cells with 0.1 μM camptothecin (CPT) compared to number of dead cells after a combinational treatment of 0.1 μM/camptothecin (CPT) and 1.0 μM peptide agent #12.

In a similar experiment, the peptide agent #12 may be found to chemopotentiate cis-platin. Combination of 0.1 μM cis-platin with 0.1 pM peptide agent #12 may be found to induce more apoptosis in T-cells that 1.0 μM of cis-platin (a 10-fold increase) by itself.

### Example 4—EMSA Assay for NF-κB-DNA Binding

Human Jurkat leukemia T-cells (clone E6-1; Amer. Type Culture Collection, Rockville, Md.) are grown in RPMI-1640 Media (Gibco-BRL, Rockville, Md.) supplemented with 10% fetal bovine serum, penicillin (614 ng/mL), streptomycin (10 μg/mL) and HEPES buffer, pH 7.2 at 37° C., 5% CO₂. The Jurkat cells (1×10⁶ cells/nL) are subsequently treated with various concentrations of the agents for 30 minutes at 37° C. and 5% CO₂ followed by PMA (50 ng/mL) and PHA (1 mM/mL) stimulation for an additional 30 minutes. The cells are harvested by centrifugation, washed in ice cold PBS and the nuclear extracts are prepared as previously described (Dignam, et al., Nucl. Acids Res 11: 1475-1489 (1983)). The protein concentration of the extracts is determined according to the Method of Bradford (1976) with BioRad reagents. Nuclear extracts are incubated for 20 min. at RT with a double stranded Cy3 labeled NF-κB consensus oligonucleotide. The binding mixture (25 mL) contains 10 mM HEPES-NaOH pH 7.9, 4 mM tris-HCl, pH 7.9, 6.0 mM KCl, 1 mM EDTA, 1 mM DTT, 10% glycerol, 0.3 mg/mL bovide serum albumin and 1 mg of poly (dI.dC). The binding mixtures (10 mg of nuclear extract protein) are incubated for 20 minutes at room temperature with 0.16 pmol of Cy3 labeled oligonucleotide. The mixture is loaded on a 4% polyacrylamide gel prepared in 1×tris borate/EDTA buffer and is electrophoresed at 200 V for 20 minutes. After electrophoresis the gel is analyzed using a phosphorimager (Biorad FX plus) for detection of the NF-κB-DNA binding.

Inhibition of translocation with p65-ELISA assay is as follows. The quantity of p65/p50 heterodimer that has translocated into the nucleus is measured using a NF-κB p65 sandwich ELISA assay (Imgenex Corp.). Jurkat cells are grown to 2×10⁶ cells/mL and treated with 50 ng/mL PMA and I g/mL PMA/PHA and incubated at 37° C., 5% CO₂. The cells are harvested after 30 minutes and nuclear extracts are prepared as previously described by Dignam and coworkers (Dignam, et al., Nucl. Acids Res 11: 1475-1489 (1983)). The NF-κB p65 sandwich ELISA kit is then used to monitor and quantify p65 translocation into the nucleus according to the manufacturer's protocol.

### Example 5—Apoptosis Assay

Induction of apoptosis using caspase 3/7 assay is as follows. CEM cells (CCRF-CEM); Amer. Type Culture Collection, Rockville, Md.) are grown in RPMI-1640 Media supplemented with 10% fetal bovine serum, penicillin (500 ng/mL), streptomycin (12 μg/mL) and hepes buffer, pH 7.2 at 37° C., 5% CO₂. DMSO is used as the vector for all drugs and added in the control experiments. Cell cultures are treated with 1 μM, 0.1 μM, 10 nM, 1 nM, 100 pM, 10 pM, 1 pM, and 0.1 pM of the agents and allowed to incubate at 37° C., 5% CO₂. An aliquote is transferred to a 96-well plate and mixed with an equal volume of Apo-ONE™ Homogenous Caspase-3/7 assay (Promega Corporation) reagent. The contents of the plate are gently mixed and allowed to incubate for 1 hour. The fluorescence of each well is then measured on a Molecular Imager FX Pro at 532 nm. All reported data is the average of two independent experiments unless otherwise indicated.

Percent Inhibition of LPS/IFNγ-induced Cytokine Expression

| | Smalt molecule | Methylated Small molecule |
|---|---|---|
| Cytokine | Agent 9 | Agent 23 |
| TNFα | 50% | 0% |
| RANTES | 43% | 0% |

Percent Inhibition of LPS/IFNγ-induced Cytokine Expression

| | Antibody | CDR3 mutant of Antibody |
|---|---|---|
| Cytokine | Agent 36 | Agent 42 |
| TNFα | 56% | 0% |
| RANTES | 41% | 0% |

Percent Inhibition of LPS/IFNγ-induced Cytokine Expression

| | -XXXXFYFXXXX- | -XXXXFYAXXXX- |
|---|---|---|
| Cytokine | Peptide Agent | Peptide Agent |
| TNFα | 67% | 3% |
| RANTES | 63% | 3% |

Percent Inhibition of LPS/IFNγ-induced Cytokine Expression

| | NNNNNUNNNN | |
|---|---|---|
| | Nucleic Acid Agent | NNNNNNNNNN |
| Cytokine | U = unnatural hydrophobic nucleic acid | Nucleic Acid Agent |
| TNFα | 57% | 1% |
| RANTES | 58% | 0% |

Approximately 10 μg of GST-AC (core enzyme) or GST-Cdc6 is bound to 10 μl of glutathione resin via GST tag. The resin is washed with 200 μl assay buffer three times to remove the excess unbound protein. 10 μg or 1 μg, 0.1 μg, 10 ng, 1 ng, 100 pg, 10 pg, 1 pg, and 0.1 pg of small molecule agents #1-10, peptide agents #11-20, nucleic acid agents #21-30, and antibody agents #31-40, wild type PR70 constructs, or mutant PR70 constructs (FYA) are added to the resin in a 200 μl volume suspended in the assay buffer containing 25 mM Tris (pH 8.0), 150 mM NaCl, 1 mM CaCl2, and 3 mM DTT. The mixture is washed three times with the assay buffer. The proteins that remain bound to resin are examined by SDS-PAGE, and visualized by Coomassie blue staining. All experiments are repeated three times. For binding of PR70 vacant of calcium, 1 mM CaCl₂ is replaced with 0.1 mM EDTA as indicated. For determination of the binding affinity, titration of small molecule agents #1-10, peptide agents #11-20, nucleic acid agents #21-30, and antibody agents #31-40, wild type PR70 constructs, or mutant PR70 constructs are added to the immobilized GST-AC for pull down. The immobilized GST is used as control. The level of binding is quantified using Image J, and results from three separate experiments are fitted in GraghPad Prism (GraphPad Software Inc.) after background subtraction to estimate K_{d}.

### Example 7—Screening Assays

The disclosure provides methods (also referred to herein as “screening assays”) for identifying polypeptides, small molecules, or bifunctional derivatives which bind to the FYF (SEQ ID NO: 2) binding region of PP2A.

The binding affinity of polypeptides that bind to the FYF (SEQ ID NO: 2) binding region of PP2A can be measured using the methods described herein, for example, by using a titration binding assay. The FYF (SEQ ID NO: 2) binding region of PP2A can be exposed to varying concentrations of a candidate agent (i.e., polypeptides, antibodies, small molecules, nucleic acids, and the like) (e.g., 1 pM, 10 pM, 100 pM, 1 nM, 10 nM, 100 nM, 1 μM, 10 μM, 100 μM, 1 mM, and 10 mM) and binding can be measured, e.g., using surface plasmon resonance to determine the Kd for binding. Additionally, the binding interactions of fluorescently-labeled agents to the FYF (SEQ ID NO: 2) binding region of PP2A can be used in a competitive binding assay to screen for and identify agents that compete with PR70 or PR72 peptides, and further calculate Ki values for binding competition. Candidate agents could also be screened for biological activity in vivo. Cell permeability screening assays in which fluorescently labeled candidate agents are applied to intact cells, which are then assayed for cellular fluorescence by microscopy. High-throughput cellular fluorescence detection can also be used.

The assays described herein can be performed with individual candidate agents or can be performed with a plurality of candidate agents. Where the assays are performed with a plurality of candidate agents, the assays can be performed using mixtures of candidate agents or can be run in parallel reactions with each reaction having a single candidate agent. The test agents or agents can be obtained using any of the numerous approaches in combinatorial library methods known in the art.

Thus, one can expose the FYF (SEQ ID NO: 2) binding region of PP2A to a test agent in the presence of a B subunit protein of PP2A, such as PR70 or PR72, and determine whether the test agent reduces (inhibits) binding of the B subunit protein to the FYF (SEQ ID NO: 2) binding region of PP2A. A test agent that inhibits binding is a candidate inhibitor of the interaction between the B subunit protein and the FYF (SEQ ID NO: 2) binding region of PP2A. Test agents can be tested for their ability to inhibit binding to the FYF (SEQ ID NO: 2) binding region of PP2A in order to identify agents that are relatively selective for inhibiting B subunit protein binding.

### Example 8—Flow Cytometry

Jurkat T-cell leukemia cells are grown in RPMI-1640 (Gibco) medium with 10% fetal bovine serum, 100 U/mL penicillin, 100 μg/mL, 2 mM glutamine, 50 mM Hepes pH 7, and 50 μM β-mercaptoethanol. SJSA-1 cells are cultured in McCoy's 5A media (ATCC) supplemented with 10% fetal bovine serum and 100 U/mL penicillin. Jurkat cells (50,000 cells per well) are treated with fluoresceinated peptide agents (10 μM) for up to 4 hours at 37° C. After washing with media, the cells are exposed to trypsin (0.25%; Gibco) digestion (30 min, 37° C.), washed with PBS, and resuspended in PBS containing 0.5 mg/mL propidium iodide. Cellular fluorescence and propidium iodide positivity are analyzed using a FACSCalibur flow cytometer and FlowJo software. The identical experiment is performed with 30 min pre-incubation of cells at 4° C. followed by 4 hour incubation with fluoresceinated peptide agents at 4° C. to assess temperature-dependence of fluorescent labeling.

### Example 9—Confocal Microscopy

Jurkat T-cell leukemia cells are incubated with fluoresceinated agents for 24 hours at 37° C. After washing with PBS, the cells are cytospun at 600 rpm for 5 minutes onto Superfrost plus glass slides (Fisher Scientific). The cells are fixed in 4% paraformaldehyde, washed with PBS, incubated with TOPRO-3 iodide (100 nM; Molecular Probes) to conterstain nuclei, treated with Vectashield mounting medium (Vector), and imaged by confocal microscopy (BioRad 1024 or Nikon E800). In a similar fashion, SJSA-1 osteosarcoma cells (1×10⁵ cells) are incubated in with fluoresceinated agents for 24 hours at 37° C. in Lab-Tek™ CC2 Chamber Slides (Nunc). After washing with PBS, the cells are fixed in 4% paraformaldehyde, washed with PBS, and treated with DAPI-containing (nuclear counterstain) Vectashield mounting medium (Vector), coverslipped and imaged by confocal microscopy (BioRad 1024 or Nikon E800). Cells are also analyzed for nuclear localization and phosphorylation status of NF-κB using commercially available antibodies.

### Example 10—Western Blotting

SJSA-1 osteosarcoma cells (1×10⁶) incubated at 37° C. are treated with 10 μM, 1 μM, 0.1 μM, 10 nM, 1 nM, 100 pM, 10 pM, 1 pM, and 0.1 pM of small molecule agents #1-10, peptide agents #11-20, nucleic acid agents #21-30, and antibody agents #31-40 in serum-free media for 4 hours, followed by serum replacement and additional incubation for 4-26 additional hours. The cells are lysed (20 mM Tris-HCl pH 8.0, 0.8% SDS, 1 mM PMSF, 1 U/mL benzonase nuclease) and the crude lysates are clarified by brief centrifugation and total protein concentration is determined by using the Pierce BCA protein assay. Aliquots containing 5 μg of total protein are run on 4-12% Bis-Tris polyacrylamide gels. Immunoprecipitations using PP2A subunit A or C antibodies are also included in the analysis. Proteins are detected by chemiluminescence reagent using antibodies specific for PR70, PR72, a B subunit of PP2A, phospho-NF-κB, and NF-κB. Release of cytokines into the media can also be measured using these methods.

### Example 11—Cell Viability and Apoptosis High-Throughput Assays

SJSA-1 osteosarcoma cells (4×10⁵ cells per well) are incubated in 96-well plates and treated with 10 μM, 1 μM, 0.1 μM, 10 nM, 1 nM, 100 pM, 10 pM, 1 pM, and 0.1 pM of small molecule agents #1-10, peptide agents #11-20, nucleic acid agents #21-30, and antibody agents #31-40 in serum-free media for 4 hours, followed by serum replacement and additional incubation for 20 hours. Cell viability is assayed by addition of CellTiter-Glo™ bioluminescence reagent (Promega) and reading luminescence in a Spectramax M5 microplate reader (Molecular Devices). The extent of apoptosis is measured through the detection of caspase-3 activity by exposing the cells to a caspase-3-specific substrate (Oncogene). Fluorescence as a result of substrate cleavage is measured in a Spectramax M5 microplate reader (Molecular Devices).

### Example 12—Co-Immunoprecipitation of Agents and PP2A Core Enzyme

SJSA-1 osteosarcoma cells (1×10⁶) are treated with 10 μM, 1 μM, 0.1 μM, 10 nM, 1 nM, 100 pM, 10 pM, 1 pM, and 0.1 pM of FITC-labeled small molecule agents #1-10, FITC-labeled peptide agents #11-20, FITC-labeled nucleic acid agents #21-30, and FITC-labeled antibody agents #31-40 in serum-free media for 4 hours, followed by serum replacement and additional 8 hour incubation. The cells are thoroughly washed with serum-containing media and PBS and exposed to lysis buffer (50 mM Tris pH 7.6, 150 mM NaCl, 1% Triton-X100, 1.0 mM PMSF, 1 U/mL benzonase nuclease and complete protease inhibitor tablet) at room temperature. All subsequent steps are all performed at 4° C. The extracts are centrifuged, and the supernatants are incubated with protein A/G sepharose. The pre-cleared supernatants (500 μL) are collected after centrifugation, incubated with 10 μL of goat-anti-FITC antibody for 1.5 h followed by protein A/G sepharose for an additional 1.5 hours. The immunoprecipitation reactions are pelleted and washed three times with lysis buffer. The precipitated proteins are suspended in SDS-containing loading buffer, boiled, and the supernatants are processed by SDS-PAGE on 4-12% Bis-Tris gels (Invitrogen). The proteins are blotted into Immobilon-P membranes (Millipore). After blocking, the blots are incubated with either a 1:100 dilution of mouse anti-human PP2A core enzyme antibody, a mouse anti-human PR70 or PR72 antibody, or a rabbit anti-FITC antibody in 3% BSA in PBS followed by anti-mouse or anti-rabbit horseradish peroxidase-conjugated IgG. The PP2A core enzyme protein, PR70 protein, PR72 protein, and FITC labeled agents are visualized using the Western Lightning™ chemiluminescence reagent (Perkin Elmer) and exposing to film. The gels are stained using a silver stain kit (Bio-Rad) following manufacturer's instructions.

### Example 13—Circular Dichroism (CD) Spectroscopy

For circular dichroism (CD) spectroscopy, agents are dissolved in H₂O to concentrations ranging from 10 μM-0.1 pM. The spectra are obtained on a Jasco J-715 spectropolarimeter at 20° C. The spectra are collected using a 0.1 cm pathlength quartz cuvette with the following measurement parameters: wavelength, 185-255 nm; step resolution 0.1 nm; speed, 20 nm min⁻¹; accumulations, 6; bandwidth, 1 nm.

### Example 14—Ex Vivo Protease Stability

To assess the protease stability of the agents, fluoresceinated agents (2.5 μg) were incubated with fresh mouse serum (20 μL) at 37° C. for 0-24 hours. The level of intact fluoresceinated agent is determined by flash freezing the serum specimens in liquid nitrogen, lyophilization, extraction in 1:1 CH₃CN:H₂O containing 0.1% TFA, followed by HPLC-based quantitation using fluorescence detection at excitation/emission settings of 495/530 nm.

### Example 15—Protein Production

Purified peptide and antibody agents are prepared as follows. Escherichia coli BL21 (DE3) containing the plasmid encoding the agent with an N-terminal hexahistidine tag and a thrombin cleavage site are cultured in kanamycin- and chloramphenicol-containing Luria Broth and induced with 0.1 mM isopropyl β-D-thiogalactoside (IPTG). The cells are harvested after 4 hours by centrifugation for 20 min at 3200 rpm, resuspended in buffer A (20 mM Tris pH 7.4, 0.5 M NaCl) and lysed by sonication. Cellular debris is pelleted by centrifugation for 30 minutes at 15,000 rpm, and the supernatant is incubated with Ni-NTA agarose for 2 h. The resin is washed with buffer A and eluted with a gradient of imidazole ranging from 5 mM to 500 mM. The fractions containing the eluted agents are concentrated and diluted 1:1 with thrombin cleavage buffer (5 mM CaCl₂, 20 mM Tris pH 7.4, 1 μL/mL β-mercaptoethanol, and 0.8 U/mL thrombin). The cleavage reaction is incubated overnight at 4° C. The reaction is concentrated to 2 mL and purified by gel filtration using a G75 column. Purity of the agent is assessed by SDS-PAGE, FPLC and MALDI-TOF and determined to be >90%. Its identity is further confirmed by digestion followed by mass spectrometry of the resulting peptide fragments.

### Example 16—Fluorescence Polarization

Fluoresceinated agents are incubated with PP2A core enzyme in binding assay buffer (140 mM NaCl, 50 mM, Tris pH 8.0) at room temperature. Binding activity is measured by fluorescence polarization on a Perkin-Elmer LS50B luminescence spectrophotometer using a cuvette containing a stirbar or a Spectramax M5 Microplate Reader (Molecular Devices). K_{d} values were determined by nonlinear regression analysis of dose response curves using Prism software 4.0 Graphpad.

### Example 17. Evaluation of Dendritic Cell (DC) Maturation

DC maturation and ChemR23 expression are measured by FACS analysis at pre and post dendritic cell maturation. Cell lysates are assayed for IL-12p35 mRNA using Luminex™ quantigene technology at 1, 3 and 6 hours post LPS/R848 stimulation. Lysates were also assayed for NF-κB by InstantOne™ ELISA at 3 and 12 hours. Agents are test at various concentrations at all time points.

DC Maturation.

Briefly 1 vial of monocytes is thawed at 37° C. and the entire contents transferred to a flask containing enough pre-warmed media to give a cell density of 0.5×10⁶ cell/cm².

FACS analysis is used to examine the expression of ChemR23 in addition to mature DC markers. On days 1 and 4, media is changed, returning any cells that are aspirated off in the media. On day 6, 1×component B is added to the media according to maturation kit directions and the cells are allowed to mature for an additional 2 days. On day 8, loosely adherent cells are harvested and a portion of the cells are used to assess DC maturation by flow cytometry. The majority of the cells are used for setting up the following assay to measure the expression of NF-κB and IL12p35.

Assay.

1×10⁵ matured DCs/well are added in 0.1 ml to the appropriate wells of the 96 well black-walled plates. Cells are allowed to settle for at least 30 minutes. The indicated test agent (10 nM final), Dexamethasone, (2 μM final) or media are added to the appropriate wells. The plates are incubated at 37° C. with 5% CO₂ for 1 hour. Next, a final LPS/R848 concentration of 0.01/2.5 μg/mL is added into the well. At the same time, an equal volume of incomplete media are added to the nontreated wells. At time points 1, 3, 6, 12 and 24 hrs, supernatants are harvested for future analysis and cells are lysed and stored at −80° C. for mRNA analysis. Time points 3 and 12 hours, cells in duplicate plates are lysed and stored at −80° C. for NF-κB analysis. After 20 hours of LPS/R848 stimulation, AlamarBlue is added to each well. The plates are incubated at 37° C. with 5% CO₂ for an additional 4 hours. At 24 hours post LPS/R848 stimulation, cell viability is assayed by reading the fluorescence of AlamarBlue. Supernatants were harvested for future analysis and cells are harvested for lysate and stored at −80° C. for mRNA analysis.

NF-κB Production.

Total NF-κB is assayed on the cell lysate at 3, 12 and 24 hrs post LPS/R848 stimulation using the InstantOne™ ELISA kit.

mRNA Production.

IL-12p35 mRNA is assayed on the cell lysate at 1, 3, 6 and 12 hrs post LPS/R848 stimulation.

DC Maturation.

On day 0 a small proportion of the cells are used to assess DC maturation by flow cytometry. The expression of the following markers is assed CD14, CD45, CD83. Mature monocyte derived DCs are expected to be CD14-CD45+CD83+. In addition the expression of the marker ChemR23 is assessed.

Cell Viability.

Cell viability following LPS stimulation is examined by AlamarBlue assay.

### Example 18—Imiquimod-Induced Psoriasis-Like Skin Inflammation Model in Mice

Psoriasis is induced by imiquimod applied to the back and ears of the test animals. The initial signs of the disease are observed within 5-6 days as redness and the appearance of plaque type psoriasis, and the disease progresses onto the entire back of the animal.

Psoriasis Clinical Score.

The study is carried out on female BALC/c mice, 9-10 weeks old at study initiation. The weight variation of the animals at the study initiation did not exceed±20% of the mean weight. The animals are divided into group 1 (naïve), group 2 (vehicle+IMQ), group 3 (C15 peptide test agent+IMQ) and group 4 (C15 peptide test agent+IMQ).

The animals are examined for signs of psoriasis on study day 1. These scores served as a baseline for the psoriasis clinical score parameter. Starting from IMQ cream application on day 1, psoriasis responses are examined from day 5 until termination of the study.

Psoriasis reactions (erythema and plaques) are scored and recorded. For plaques, a scale of 0-7 is used with 0 being normal and 7 being no fur with small red dots covering 50%-100% of the back. For erythema, a scale of 0-4 is used with 0 being normal and 4 being very marked. The clinical score is determined by summing the score of each section. The final psoriasis score is the sum of the erythema score and the plaques score.

Ear Thickness.

Right ear thickness is measured before psoriasis induction on study day 1 (baseline). These measurements served as a baseline for the ear thickness parameter. Right ear thickness is then measured on study days 5, 8 and 10 (study termination). The measurements are carried out using a digital caliper.

General Clinical Signs.

Throughout the 10-day study, general clinical signs and observation were performed and recorded if any unexpected abnormalities are observed.

Body Weights.

Individual body weights are determined shortly before psoriasis induction on study day 1. These weights are used as baseline measurements. From disease induction the animals are weighed on day 5 and on study termination day 10.

Blood Collection for Serum.

500 μl of whole blood via retro orbital bleeding is taken for serum analysis on study day 3 and on study day 10. Blood is collected in the 1.5 ml tubes without EDTA. After a wait time of 0.5 hour, the sample is centrifuge for 5 minutes in 3000 rpm. Serum is collected using filtered pipette tips and stored at −80° C.

Organ Collection.

At study termination 4 hours after IMQ application, mice are culled with CO₂. Groups are divided into two cohorts as follows: Cohort 1: 3 animals.

1. A method for controlling cellular expression of a gene, the method comprising contacting a cell with an effective amount of an agent that;
i) maintains NF-κB activity in the cell at a resting or baseline level or inhibits an undesired increase in NF-κB activity; and/or
ii) antagonizes or regulates the formation of PP2A holoenzyme; and/or
iii) stabilizes a complex of PP2A core enzyme and proteins in the NF-κB pathway.

2. A method of treating an inflammatory disorder in an individual in need thereof, comprising administering to the individual an effective amount of an agent, wherein the agent is administered in an amount sufficient to;
i) maintain NF-κB activity in a cell at a resting or baseline level or to inhibit an undesired increase in NF-κB activity and/or
ii) antagonize or regulate the formation of PP2A holoenzyme; and/or
iii) stabilize a complex of PP2A core enzyme and proteins in the NF-κB pathway.

3. The method of claim 2 wherein the inflammatory disorder is responsive to treatment with a glucocorticoid and/or with dexamethasone.

4. The method of claim 2 wherein the inflammatory disorder is selected from the group consisting of psoriasis, atopic dermatitis, contact dermatitis, lichen planus, acne, alopecia areata, IBD, Crohn's Disease and/or ulcerative colitis, uveitis, dry eye, blepharitis, allergic conjunctivitis, iritis, a retinal inflammatory disease, and any combination thereof.

5. The method of claim 2 wherein the inflammatory disorder is a retinal inflammatory disease that is AMD.

6. The method of claim 2 wherein the inflammatory disorder is a retinal inflammatory disease that is DME.

7. The method of claim 2 wherein the inflammatory disorder is selected from the group consisting of acute disseminated encephalomyelitis; Addison's disease; ankylosing spondylitis; antiphospholipid antibody syndrome; autoimmune hemolytic anemia; autoimmune hepatitis; autoimmune inner ear disease; bullous pemphigoid; Chagas disease; chronic obstructive pulmonary disease; coeliac disease; dermatomyositis; diabetes mellitus type 1; diabetes mellitus type 2; endometriosis; Goodpasture's syndrome; Graves' disease; Guillain-Barre syndrome; Hashimoto's disease; idiopathic thrombocytopenic purpura; interstitial cystitis; systemic lupus erythematosus (SLE); metabolic syndrome; multiple sclerosis; Myasthenia gravis; myocarditis; narcolepsy; obesity; pemphigus vulgaris; pernicious anaemia; polymyositis; primary biliary cirrhosis; rheumatoid arthritis; schizophrenia; scleroderma; Sjogren's syndrome; vasculitis; vitiligo; Wegener's granulomatosis; allergic rhinitis; prostate cancer; non-small cell lung carcinoma; ovarian cancer; breast cancer; melanoma; gastric cancer; colorectal cancer; brain cancer; metastatic bone disorder; pancreatic cancer; a lymphoma; nasal polyps; gastrointestinal cancer; ulcerative colitis; Crohn's disorder; collagenous colitis; lymphocytic colitis; ischaemic colitis; diversion colitis; Behcet's syndrome; infective colitis; indeterminate colitis; inflammatory liver disorder; endotoxin shock; rheumatoid spondylitis; ankylosing spondylitis; gouty arthritis; polymyalgia rheumatic; Alzheimer's disorder; Parkinson's disorder; epilepsy; AIDS dementia; asthma; adult respiratory distress syndrome; bronchitis; cystic fibrosis; acute leukocyte-mediated lung injury; distal proctitis; Wegener's granulomatosis; fibromyalgia; uveitis; conjunctivitis; psoriasis; eczema; dermatitis; smooth muscle proliferation disorders; meningitis; shingles; encephalitis; nephritis; tuberculosis; retinitis: atopic dermatitis; pancreatitis; periodontal gingivitis; coagulative necrosis; liquefactive necrosis; fibrinoid necrosis; hyperacute transplant rejection; acute transplant rejection; chronic transplant rejection; acute graft-versus-host disease; chronic graft-versus-host disease; abdominal aortic aneurysm (AAA); and any combination thereof.

8. The method of claim 1 wherein the gene is selected from the group consisting of TNFα, IL-6, IL-12, IL-17, IL-23, and combinations thereof, or from the group consisting of IL-1, IL-2, IL-3, IL-4, IL-5, IL-6, IL-7, IL-8, IL-9, IL-11, IL-12, IL-13, IL-14, IL-15, IL-16, IL-17, IL-18, IL-19, IL-20, IL-21, TL-22, IL-23, IL-24, 11-25, IL-26, IL-27, IL-28, IL-29, IL-30, a TNF family member, an IFN family member, MCP-1, MIP-1, and any combination thereof.

9. The method of claim 1 wherein the agent is a small molecule, an antibody, a nucleic acid or a peptide.

10. The method of claim 2 wherein the agent is a small molecule, an antibody, a nucleic acid or a peptide.

11. The method of claim 1 wherein the agent is a peptide comprising the amino acid sequence FYF; or the amino acid sequence FYFP; or the amino acid sequence PFYFP; or the amino acid sequence PXFYFP, wherein X is any amino acid or analog thereof; or the amino acid sequence PXXFYFP, wherein X is any amino acid or analog thereof; or the amino acid sequence PSFYFP; or the amino acid sequence PTFYFP; or the amino acid sequence PX(S/T)FYFP; or the amino acid sequence PHSFYFP; or the amino acid sequence PHTFYFP.

12. The method of claim 2 wherein the agent is a peptide comprising the amino acid sequence FYF; or the amino acid sequence FYFP; or the amino acid sequence PFYFP; or the amino acid sequence PXFYFP, wherein X is any amino acid or analog thereof; or the amino acid sequence PXXFYFP, wherein X is any amino acid or analog thereof; or the amino acid sequence PSFYFP; or the amino acid sequence PTFYFP; or the amino acid sequence PX(S/T)FYFP; or the amino acid sequence PHSFYFP; or the amino acid sequence PHTFYFP.

13. The method of claim 1 wherein the agent is a peptide comprising a nuclear translocation signal sequence.

14. The method of claim 13, where in the nuclear translocation signal sequence comprises a gapped dipeptide sequence.

15. The method of claim 2 wherein the agent is a peptide comprising a nuclear translocation signal sequence.

16. The method of claim 15, where in the nuclear translocation signal sequence comprises a gapped dipeptide sequence.

17. The method of claim 1 further comprising administering the agent before, after, or simultaneously with an anti-inflammatory agent.

18. The method of claim 17, wherein the anti-inflammatory agent is selected from the group consisting of an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent an alkylating agent, an anti-metabolite, a plant alkaloid, a terpenoids, a topoisomerase inhibitor, an antitumor antibiotic, an antibody, a hormonal therapy, an anti-diabetes agent, a leukotriene inhibitor, and any combination thereof.

19. The method of claim 1 further comprising administering the agent before, after, or simultaneously with an anti-inflammatory agent.

20. The method of claim 19, wherein the anti-inflammatory agent is selected from the group consisting of an anti-TNF agent, an IL-1 receptor antagonist, an IL-2 receptor antagonist, a cytotoxic agent, an immunomodulatory agent, an antibiotic, a T-cell co-stimulatory blocker, a B cell depleting agent, an immunosuppressive agent an alkylating agent, an anti-metabolite, a plant alkaloid, a terpenoids, a topoisomerase inhibitor, an antitumor antibiotic, an antibody, a hormonal therapy, an anti-diabetes agent, a leukotriene inhibitor, and any combination thereof.

21.-188. (canceled)


SEQUENCE LISTING (266 sequences)

SEQ ID NO: 1 - protein, 15 aa, Homo sapiens
AGEDPHSFYFPGQFA

SEQ ID NO: 2 - protein, 3 aa, Artificial Sequence
  Description of Artificial Sequence Synthetic peptide
FYF

SEQ ID NO: 3 - protein, 4 aa, Artificial Sequence
  Description of Artificial Sequence Synthetic peptide
FYFP

SEQ ID NO: 4 - protein, 5 aa, Artificial Sequence
  Description of Artificial Sequence Synthetic peptide
PFYFP

SEQ ID NO: 5 - protein, 6 aa, Artificial Sequence
  Description of Artificial Sequence Synthetic peptide
  MOD_RES (2)..(2): Any amino acid or amino acid analog
PXFYFP

SEQ ID NO: 6 - protein, 6 aa, Artificial Sequence
  Description of Artificial Sequence Synthetic peptide
  MOD_RES (2)..(2): Ser or Thr
PXFYFP

SEQ ID NO: 7 - protein, 6 aa, Artificial Sequence
  Description of Artificial Sequence Synthetic peptide
PSFYFP

SEQ ID NO: 8 - protein, 6 aa, Artificial Sequence
  Description of Artificial Sequence Synthetic peptide
PTFYFP

SEQ ID NO: 9 - protein, 7 aa, Artificial Sequence
  Description of Artificial Sequence Synthetic peptide
  MOD_RES (2)..(2): Any amino acid or amino acid analog
  MOD_RES (3)..(3): Ser or Thr
PXXFYFP

SEQ ID NO: 10 - protein, 7 aa, Artificial Sequence
  Description of Artificial Sequence Synthetic peptide
PHSFYFP

SEQ ID NO: 11 - protein, 7 aa, Artificial Sequence
  Description of Artificial Sequence Synthetic peptide
PHTFYFP

SEQ ID NO: 12 - protein, 2 aa, Artificial Sequence
  Description of Artificial Sequence Synthetic peptide
GP

SEQ ID NO: 13 - protein, 2 aa, Artificial Sequence
  Description of Artificial Sequence Synthetic peptide
DS

SEQ ID NO: 14 - protein, 2 aa, Artificial Sequence
  Description of Artificial Sequence Synthetic peptide
PS

SEQ ID NO: 15 - protein, 2 aa, Artificial Sequence
  Description of Artificial Sequence Synthetic peptide
PP

SEQ ID NO: 16 - protein, 2 aa, Artificial Sequence
  Description of Artificial Sequence Synthetic peptide
PG

SEQ ID NO: 17 - protein, 4 aa, Artificial Sequence
  Description of Artificial Sequence Synthetic peptide
  MOD_RES (2)..(3): Any amino acid
GXXP

SEQ ID NO: 18 - protein, 4 aa, Artificial Sequence
  Description of Artificial Sequence Synthetic peptide
  MOD_RES (2)..(3): Any amino acid
DXXS

SEQ ID NO: 19 - protein, 3 aa, Artificial Sequence
  Description of Artificial Sequence Synthetic peptide
  MOD_RES (2)..(2): Any amino acid
PXS

SEQ ID NO: 20 - protein, 7 aa, Artificial Sequence
  Description of Artificial Sequence Synthetic peptide
  MOD_RES (2)..(6): Any amino acid
PXXXXXP

SEQ ID NO: 21 - protein, 6 aa, Artificial Sequence
  Description of Artificial Sequence Synthetic peptide
  MOD_RES (2)..(3): Any amino acid
  MOD_RES (5)..(5): Any amino acid
GXXPXS

SEQ ID NO: 22 - protein, 6 aa, Artificial Sequence
  Description of Artificial Sequence Synthetic peptide
  MOD_RES (2)..(2): Any amino acid
  MOD_RES (5)..(5): Any amino acid
GXDPXS

SEQ ID NO: 23 - protein, 11 aa, Artificial Sequence
  Description of Artificial Sequence Synthetic peptide
  MOD_RES (2)..(3): Any amino acid
  MOD_RES (5)..(9): Any amino acid
GXXPXXXXXPG

SEQ ID NO: 24 - protein, 10 aa, Artificial Sequence
  Description of Artificial Sequence Synthetic peptide
  MOD_RES (2)..(3): Any amino acid
  MOD_RES (5)..(9): Any amino acid
GXXPXXXXXP

SEQ ID NO: 25 - protein, 4 aa, Artificial Sequence
  Description of Artificial Sequence Synthetic peptide
  MOD_RES (3)..(3): Any amino acid
DPXS

SEQ ID NO: 26 - protein, 8 aa, Artificial Sequence
  Description of Artificial Sequence Synthetic peptide
  MOD_RES (3)..(3): Any amino acid
  MOD_RES (5)..(7): Any amino acid
DPXSXXXP

SEQ ID NO: 27 - protein, 9 aa, Artificial Sequence
  Description of Artificial Sequence Synthetic peptide
  MOD_RES (2)..(3): Any amino acid
  MOD_RES (5)..(7): Any amino acid
DXXSXXXPG

SEQ ID NO: 28 - protein, 7 aa, Artificial Sequence
  Description of Artificial Sequence Synthetic peptide
  MOD_RES (2)..(2): Any amino acid
  MOD_RES (4)..(6): Any amino acid
PXSXXXP

SEQ ID NO: 29 - protein, 8 aa, Artificial Sequence
  Description of Artificial Sequence Synthetic peptide
  MOD_RES (2)..(2): Any amino acid
  MOD_RES (4)..(6): Any amino acid
PXSXXXPG

SEQ ID NO: 30 - protein, 11 aa, Artificial Sequence
  Description of Artificial Sequence Synthetic peptide
  MOD_RES (2)..(2): Any amino acid
  MOD_RES (5)..(5): Any amino acid
  MOD_RES (7)..(9): Any amino acid
GXDPXSXXXPG

SEQ ID NO: 31 - protein, 15 aa, Artificial Sequence
  Description of Artificial Sequence Synthetic peptide
  MOD_RES (1)..(1): Any amino acid
  MOD_RES (3)..(3): Any amino acid
  MOD_RES (6)..(6): Any amino acid
  MOD_RES (8)..(10): Any amino acid
  MOD_RES (13)..(15): Any amino acid
XGXDPXSXXXPGXXX

SEQ ID NO: 32 - protein, 15 aa, Artificial Sequence
  Description of Artificial Sequence Synthetic peptide
LSINIPRFYFPEGLP

SEQ ID NO: 33 - protein, 16 aa, Artificial Sequence
  Description of Artificial Sequence Synthetic peptide
  MOD_RES (8)..(8): Any amino acid
LSINIPRXFYFPEGLP

SEQ ID NO: 34 - protein, 16 aa, Artificial Sequence
  Description of Artificial Sequence Synthetic peptide
  MOD_RES (7)..(7): Any amino acid
LSINIPXRFYFPEGLP

SEQ ID NO: 35 - protein, 15 aa, Artificial Sequence
  Description of Artificial Sequence Synthetic peptide
TSQSIPTFYFPRGRP

SEQ ID NO: 36 - protein, 16 aa, Artificial Sequence
  Description of Artificial Sequence Synthetic peptide
  MOD_RES (7)..(7): Any amino acid
TSQSIPXTFYFPRGRP

SEQ ID NO: 37 - protein, 16 aa, Artificial Sequence
  Description of Artificial Sequence Synthetic peptide
  MOD_RES (8)..(8): Any amino acid
TSQSIPTXFYFPRGRP

SEQ ID NO: 38 - protein, 15 aa, Artificial Sequence
  Description of Artificial Sequence Synthetic peptide
AGEDPHGYFLPGQFA

SEQ ID NO: 39 - protein, 16 aa, Artificial Sequence
  Description of Artificial Sequence Synthetic peptide
AGEDPHSFYFPGQFAF

SEQ ID NO: 40 - protein, 17 aa, Artificial Sequence
  Description of Artificial Sequence Synthetic peptide
AGEDPHSFYFPGQFAFS

SEQ ID NO: 41 - protein, 11 aa, Artificial Sequence
  Description of Artificial Sequence Synthetic peptide
PHSFYFPGQFA

SEQ ID NO: 42 - protein, 15 aa, Artificial Sequence
  Description of Artificial Sequence Synthetic peptide
  MOD_RES (1)..(1): Any amino acid or absent
XGEDPHSFYFPGQFA

SEQ ID NO: 43 - protein, 15 aa, Artificial Sequence
  Description of Artificial Sequence Synthetic peptide
  MOD_RES (2)..(2): Any amino acid or absent
AXEDPHSFYFPGQFA

SEQ ID NO: 44 - protein, 15 aa, Artificial Sequence
  Description of Artificial Sequence Synthetic peptide
  MOD_RES (3)..(3): Any amino acid or absent
AGXDPHSFYFPGQFA

SEQ ID NO: 45 - protein, 15 aa, Artificial Sequence
  Description of Artificial Sequence Synthetic peptide
  MOD_RES (4)..(4): Any amino acid or absent
AGEXPHSFYFPGQFA

SEQ ID NO: 46 - protein, 15 aa, Artificial Sequence
  Description of Artificial Sequence Synthetic peptide
  MOD_RES (5)..(5): Any amino acid or absent
AGEDXHSFYFPGQFA

SEQ ID NO: 47 - protein, 15 aa, Artificial Sequence
  Description of Artificial Sequence Synthetic peptide
  MOD_RES (6)..(6): Any amino acid or absent
AGEDPXSFYFPGQFA

SEQ ID NO: 48 - protein, 15 aa, Artificial Sequence
  Description of Artificial Sequence Synthetic peptide
  MOD_RES (7)..(7): Any amino acid or absent
AGEDPHXFYFPGQFA

SEQ ID NO: 49 - protein, 15 aa, Artificial Sequence
  Description of Artificial Sequence Synthetic peptide
  MOD_RES (8)..(8): Any amino acid or absent
AGEDPHSXYFPGQFA

SEQ ID NO: 50 - protein, 15 aa, Artificial Sequence
  Description of Artificial Sequence Synthetic peptide
  MOD_RES (9)..(9): Any amino acid or absent
AGEDPHSFXFPGQFA

SEQ ID NO: 51 - protein, 15 aa, Artificial Sequence
  Description of Artificial Sequence Synthetic peptide
  MOD_RES (10)..(10): Any amino acid or absent
AGEDPHSFYXPGQFA

SEQ ID NO: 52 - protein, 15 aa, Artificial Sequence
  Description of Artificial Sequence Synthetic peptide
  MOD_RES (11)..(11): Any amino acid or absent
AGEDPHSFYFXGQFA

SEQ ID NO: 53 - protein, 15 aa, Artificial Sequence
  Description of Artificial Sequence Synthetic peptide
  MOD_RES (12)..(12): Any amino acid or absent
AGEDPHSFYFPXQFA

SEQ ID NO: 54 - protein, 15 aa, Artificial Sequence
  Description of Artificial Sequence Synthetic peptide
  MOD_RES (13)..(13): Any amino acid or absent
AGEDPHSFYFPGXFA

SEQ ID NO: 55 - protein, 15 aa, Artificial Sequence
  Description of Artificial Sequence Synthetic peptide
  MOD_RES (14)..(14): Any amino acid or absent
AGEDPHSFYFPGQXA

SEQ ID NO: 56 - protein, 15 aa, Artificial Sequence
  Description of Artificial Sequence Synthetic peptide
  MOD_RES (15)..(15): Any amino acid or absent
AGEDPHSFYFPGQFX

SEQ ID NO: 57 - protein, 14 aa, Artificial Sequence
  Description of Artificial Sequence Synthetic peptide
  MOD_RES (8)..(8): Any amino acid or absent
  MOD_RES (10)..(10): Any amino acid or absent
AGEDPHSXYXGQFA

SEQ ID NO: 58 - protein, 13 aa, Artificial Sequence
  Description of Artificial Sequence Synthetic peptide
  MOD_RES (8)..(9): Any amino acid or absent
AGEDPHSXXGQFA

SEQ ID NO: 59 - protein, 15 aa, Artificial Sequence
  Description of Artificial Sequence Synthetic peptide
  MOD_RES (1)..(1): Any unnatural amino acid
XGEDPHSFYFPGQFA

SEQ ID NO: 60 - protein, 15 aa, Artificial Sequence
  Description of Artificial Sequence Synthetic peptide
  MOD_RES (2)..(2): Any unnatural amino acid
AXEDPHSFYFPGQFA

SEQ ID NO: 61 - protein, 15 aa, Artificial Sequence
  Description of Artificial Sequence Synthetic peptide
  MOD_RES (3)..(3): Any unnatural amino acid
AGXDPHSFYFPGQFA

SEQ ID NO: 62 - protein, 15 aa, Artificial Sequence
  Description of Artificial Sequence Synthetic peptide
  MOD_RES (4)..(4): Any unnatural amino acid
AGEXPHSFYFPGQFA

SEQ ID NO: 63 - protein, 15 aa, Artificial Sequence
  Description of Artificial Sequence Synthetic peptide
  MOD_RES (5)..(5): Any unnatural amino acid
AGEDXHSFYFPGQFA

SEQ ID NO: 64 - protein, 15 aa, Artificial Sequence
  Description of Artificial Sequence Synthetic peptide
  MOD_RES (6)..(6): Any unnatural amino acid
AGEDPXSFYFPGQFA

SEQ ID NO: 65 - protein, 15 aa, Artificial Sequence
  Description of Artificial Sequence Synthetic peptide
  MOD_RES (7)..(7): Any unnatural amino acid
AGEDPHXFYFPGQFA

SEQ ID NO: 66 - protein, 15 aa, Artificial Sequence
  Description of Artificial Sequence Synthetic peptide
  MOD_RES (8)..(8): Any unnatural amino acid
AGEDPHSXYFPGQFA

SEQ ID NO: 67 - protein, 15 aa, Artificial Sequence
  Description of Artificial Sequence Synthetic peptide
  MOD_RES (9)..(9): Any unnatural amino acid
AGEDPHSFXFPGQFA

SEQ ID NO: 68 - protein, 15 aa, Artificial Sequence
  Description of Artificial Sequence Synthetic peptide
  MOD_RES (10)..(10): Any unnatural amino acid
AGEDPHSFYXPGQFA

SEQ ID NO: 69 - protein, 15 aa, Artificial Sequence
  Description of Artificial Sequence Synthetic peptide
  MOD_RES (11)..(11): Any unnatural amino acid
AGEDPHSFYFXGQFA

SEQ ID NO: 70 - protein, 15 aa, Artificial Sequence
  Description of Artificial Sequence Synthetic peptide
  MOD_RES (12)..(12): Any unnatural amino acid
AGEDPHSFYFPXQFA

SEQ ID NO: 71 - protein, 15 aa, Artificial Sequence
  Description of Artificial Sequence Synthetic peptide
  MOD_RES (13)..(13): Any unnatural amino acid
AGEDPHSFYFPGXFA

SEQ ID NO: 72 - protein, 15 aa, Artificial Sequence
  Description of Artificial Sequence Synthetic peptide
  MOD_RES (14)..(14): Any unnatural amino acid
AGEDPHSFYFPGQXA

SEQ ID NO: 73 - protein, 15 aa, Artificial Sequence
  Description of Artificial Sequence Synthetic peptide
  MOD_RES (15)..(15): Any unnatural amino acid
AGEDPHSFYFPGQFX

SEQ ID NO: 74 - protein, 14 aa, Artificial Sequence
  Description of Artificial Sequence Synthetic peptide
  MOD_RES (8)..(8): Any unnatural amino acid
  MOD_RES (10)..(10): Any unnatural amino acid
AGEDPHSXYXGQFA

SEQ ID NO: 75 - protein, 13 aa, Artificial Sequence
  Description of Artificial Sequence Synthetic peptide
  MOD_RES (8)..(9): Any unnatural amino acid
AGEDPHSXXGQFA

SEQ ID NO: 76 - protein, 11 aa, Artificial Sequence
  Description of Artificial Sequence Synthetic peptide
PHGYFLPGQPA

SEQ ID NO: 77 - protein, 12 aa, Artificial Sequence
  Description of Artificial Sequence Synthetic peptide
PHGYFLPGQFAF

SEQ ID NO: 78 - protein, 13 aa, Artificial Sequence
  Description of Artificial Sequence Synthetic peptide
PHGYFLPGQFAFS

SEQ ID NO: 79 - protein, 16 aa, Artificial Sequence
  Description of Artificial Sequence Synthetic peptide
AGEDPHGYFLPGQFAF

SEQ ID NO: 80 - protein, 17 aa, Artificial Sequence
  Description of Artificial Sequence Synthetic peptide
AGEDPHGYFLPGQFAFS

SEQ ID NO: 81 - protein, 12 aa, Artificial Sequence
  Description of Artificial Sequence Synthetic peptide
DPHGYFLPGQFA

SEQ ID NO: 82 - protein, 13 aa, Artificial Sequence
  Description of Artificial Sequence Synthetic peptide
EDPHGYFLPGQFA

SEQ ID NO: 83 - protein, 14 aa, Artificial Sequence
  Description of Artificial Sequence Synthetic peptide
GEDPHGYPLPGQFA

SEQ ID NO: 84 - protein, 13 aa, Artificial Sequence
  Description of Artificial Sequence Synthetic peptide
DPHGYFLPGQFAF

SEQ ID NO: 85 - protein, 14 aa, Artificial Sequence
  Description of Artificial Sequence Synthetic peptide
EDPHGYFLPGQFAF

SEQ ID NO: 86 - protein, 15 aa, Artificial Sequence
  Description of Artificial Sequence Synthetic peptide
GEDPHGYFLPGQFAF

SEQ ID NO: 87 - protein, 14 aa, Artificial Sequence
  Description of Artificial Sequence Synthetic peptide
DPHGYFLPGQFAFS

SEQ ID NO: 88 - protein, 15 aa, Artificial Sequence
  Description of Artificial Sequence Synthetic peptide
EDPHGYFLPGQFAFS

SEQ ID NO: 89 - protein, 16 aa, Artificial Sequence
  Description of Artificial Sequence Synthetic peptide
GEDPHGYFLPGQFAFS

SEQ ID NO: 90 - protein, 11 aa, Artificial Sequence
  Description of Artificial Sequence Synthetic peptide
PHSEYFPGQFA

SEQ ID NO: 91 - protein, 12 aa, Artificial Sequence
  Description of Artificial Sequence Synthetic peptide
PHSFYFPGQFAF

SEQ ID NO: 92 - protein, 13 aa, Artificial Sequence
  Description of Artificial Sequence Synthetic peptide
PHSFYFPGQFAFS

SEQ ID NO: 93 - protein, 16 aa, Artificial Sequence
  Description of Artificial Sequence Synthetic peptide
AGEDPHSFYPPGQFAF

SEQ ID NO: 94 - protein, 12 aa, Artificial Sequence
  Description of Artificial Sequence Synthetic peptide
DPHSFYFPGQFA

SEQ ID NO: 95 - protein, 13 aa, Artificial Sequence
  Description of Artificial Sequence Synthetic peptide
EDPHSFYFPGQFA

SEQ ID NO: 96 - protein, 14 aa, Artificial Sequence
  Description of Artificial Sequence Synthetic peptide
GEDPHSPYFPGQFA

SEQ ID NO: 97 - protein, 13 aa, Artificial Sequence
  Description of Artificial Sequence Synthetic peptide
DPHSFYPPGQFAF

SEQ ID NO: 98 - protein, 14 aa, Artificial Sequence
  Description of Artificial Sequence Synthetic peptide
EDPHSFYFPGQFAF

SEQ ID NO: 99 - protein, 15 aa, Artificial Sequence
  Description of Artificial Sequence Synthetic peptide
GEDPHSFYFPGQFAF

SEQ ID NO: 100 - protein, 14 aa, Artificial Sequence
  Description of Artificial Sequence Synthetic peptide
DPHSFYFPGQFAPS

SEQ ID NO: 101 - protein, 15 aa, Artificial Sequence
  Description of Artificial Sequence Synthetic peptide
EDPHSFYFPGQFAFS

SEQ ID NO: 102 - protein, 16 aa, Artificial Sequence
  Description of Artificial Sequence Synthetic peptide
GEDPHSFYFPGQFAFS

SEQ ID NO: 103 - protein, 19 aa, Artificial Sequence
  Description of Artificial Sequence Synthetic peptide
AQAGEDPHGYFLPGQFAFS

SEQ ID NO: 104 - protein, 19 aa, Artificial Sequence
  Description of Artificial Sequence Synthetic peptide
QRAGEDPHSFYFPGQFAFS

SEQ ID NO: 105 - protein, 11 aa, Artificial Sequence
  Description of Artificial Sequence Synthetic peptide
PHGYFLPGQPA

SEQ ID NO: 106 - protein, 7 aa, Artificial Sequence
  Description of Artificial Sequence Synthetic peptide
  MOD_RES (2)..(3): Any amino acid or amino acid analog
PXXFYFP

SEQ ID NO: 107 - protein, 7 aa, Artificial Sequence
  Description of Artificial Sequence Synthetic peptide
  MOD_RES (2)..(2): Any amino acid or amino acid analog
PXSFYFP

SEQ ID NO: 108 - protein, 7 aa, Artificial Sequence
  Description of Artificial Sequence Synthetic peptide
  MOD_RES (2)..(2): Any amino acid or amino acid analog
PXTFYFP

SEQ ID NO: 109 - protein, 3 aa, Artificial Sequence
  Description of Artificial Sequence Synthetic peptide
  MOD_RES (2)..(2): Any amino acid
  MOD_RES (3)..(3): Phe or Tyr
FXX

SEQ ID NO: 110 - protein, 4 aa, Artificial Sequence
  Description of Artificial Sequence Synthetic peptide
  MOD_RES (1)..(1): Any amino acid or amino acid analog
XFYF

SEQ ID NO: 111 - protein, 4 aa, Artificial Sequence
  Description of Artificial Sequence Synthetic peptide
  MOD_RES (4)..(4): Any amino acid or amino acid analog
FYFX

SEQ ID NO: 112 - protein, 5 aa, Artificial Sequence
  Description of Artificial Sequence Synthetic peptide
  MOD_RES (1)..(1): Any amino acid or amino acid analog
  MOD_RES (5)..(5): Any amino acid or amino acid analog
XFYFX

SEQ ID NO: 113 - protein, 5 aa, Artificial Sequence
  Description of Artificial Sequence Synthetic peptide
  MOD_RES (1)..(1): Any amino acid or amino acid analog
XFYFP

SEQ ID NO: 114 - protein, 5 aa, Artificial Sequence
  Description of Artificial Sequence Synthetic peptide
  MOD_RES (1)..(2): Any amino acid or amino acid analog
XXFYF

SEQ ID NO: 115 - protein, 5 aa, Artificial Sequence
  Description of Artificial Sequence Synthetic peptide
  MOD_RES (2)..(2): Any amino acid or amino acid analog
PXFYF

SEQ ID NO: 116 - protein, 5 aa, Artificial Sequence
  Description of Artificial Sequence Synthetic peptide
  MOD_RES (4)..(5): Any amino acid or amino acid analog
FYFXX

SEQ ID NO: 117 - protein, 5 aa, Artificial Sequence
  Description of Artificial Sequence Synthetic peptide
  MOD_RES (5)..(5): Any amino acid or amino acid analog
FYFPX

SEQ ID NO: 118 - protein, 6 aa, Artificial Sequence
  Description of Artificial Sequence Synthetic peptide
  MOD_RES (1)..(2): Any amino acid or amino acid analog
  MOD_RES (6)..(6): Any amino acid or amino acid analog
XXFYFX

SEQ ID NO: 119 - protein, 6 aa, Artificial Sequence
  Description of Artificial Sequence Synthetic peptide
  MOD_RES (2)..(2): Any amino acid or amino acid analog
  MOD_RES (6)..(6): Any amino acid or amino acid analog
PXFYFX

SEQ ID NO: 120 - protein, 6 aa, Artificial Sequence
  Description of Artificial Sequence Synthetic peptide
  MOD_RES (1)..(2): Any amino acid or amino acid analog
XXFYFP

SEQ ID NO: 121 - protein, 6 aa, Artificial Sequence
  Description of Artificial Sequence Synthetic peptide
  MOD_RES (1)..(3): Any amino acid or amino acid analog
XXXFYF

SEQ ID NO: 122 - protein, 6 aa, Artificial Sequence
  Description of Artificial Sequence Synthetic peptide
  MOD_RES (1)..(1): Any amino acid or amino acid analog
  MOD_RES (3)..(3): Any amino acid or amino acid analog
XPXFYF

SEQ ID NO: 123 - protein, 6 aa, Artificial Sequence
  Description of Artificial Sequence Synthetic peptide
  MOD_RES (2)..(3): Any amino acid or amino acid analog
PXXFYF

SEQ ID NO: 124 - protein, 7 aa, Artificial Sequence
  Description of Artificial Sequence Synthetic peptide
  MOD_RES (1)..(3): Any amino acid or amino acid analog
  MOD_RES (7)..(7): Any amino acid or amino acid analog
XXXFYFX

SEQ ID NO: 125 - protein, 7 aa, Artificial Sequence
  Description of Artificial Sequence Synthetic peptide
  MOD_RES (1)..(3): Any amino acid or amino acid analog
XXXFYFP

SEQ ID NO: 126 - protein, 7 aa, Artificial Sequence
  Description of Artificial Sequence Synthetic peptide
  MOD_RES (2)..(3): Any amino acid or amino acid analog
  MOD_RES (7)..(7): Any amino acid or amino acid analog
PXXFYFX

SEQ ID NO: 127 - protein, 7 aa, Artificial Sequence
  Description of Artificial Sequence Synthetic peptide
  MOD_RES (1)..(1): Any amino acid or amino acid analog
  MOD_RES (3)..(3): Any amino acid or amino acid analog
  MOD_RES (7)..(7): Any amino acid or amino acid analog
XPXFYFX

SEQ ID NO: 128 - protein, 7 aa, Artificial Sequence
  Description of Artificial Sequence Synthetic peptide
  MOD_RES (1)..(1): Any amino acid or amino acid analog
  MOD_RES (3)..(3): Any amino acid or amino acid analog
XPXFYFP

SEQ ID NO: 129 - protein, 8 aa, Artificial Sequence
  Description of Artificial Sequence Synthetic peptide
  MOD_RES (1)..(3): Any amino acid or amino acid analog
  MOD_RES (7)..(8): Any amino acid or amino acid analog
XXXFYFXX

SEQ ID NO: 130 - protein, 8 aa, Artificial Sequence
  Description of Artificial Sequence Synthetic peptide
  MOD_RES (2)..(3): Any amino acid or amino acid analog
  MOD_RES (7)..(8): Any amino acid or amino acid analog
PXXFYFXX

SEQ ID NO: 131 - protein, 8 aa, Artificial Sequence
  Description of Artificial Sequence Synthetic peptide
  MOD_RES (1)..(1): Any amino acid or amino acid analog
  MOD_RES (3)..(3): Any amino acid or amino acid analog
  MOD_RES (7)..(8): Any amino acid or amino acid analog
XPXFYFXX

SEQ ID NO: 132 - protein, 8 aa, Artificial Sequence
  Description of Artificial Sequence Synthetic peptide
  MOD_RES (1)..(3): Any amino acid or amino acid analog
  MOD_RES (8)..(8): Any amino acid or amino acid analog
XXXFYFPX

SEQ ID NO: 133 - protein, 8 aa, Artificial Sequence
  Description of Artificial Sequence Synthetic peptide
  MOD_RES (2)..(3): Any amino acid or amino acid analog
  MOD_RES (8)..(8): Any amino acid or amino acid analog
PXXFYFPX

SEQ ID NO: 134 - protein, 8 aa, Artificial Sequence
  Description of Artificial Sequence Synthetic peptide
  MOD_RES (1)..(1): Any amino acid or amino acid analog
  MOD_RES (3)..(3): Any amino acid or amino acid analog
  MOD_RES (8)..(8): Any amino acid or amino acid analog
XPXFYFPX

SEQ ID NO: 135 - protein, 4 aa, Artificial Sequence
  Description of Artificial Sequence Synthetic peptide
  MOD_RES (1)..(1): Ser or Thr
XFYF

SEQ ID NO: 136 - protein, 5 aa, Artificial Sequence
  Description of Artificial Sequence Synthetic peptide
  MOD_RES (1)..(1): Ser or Thr
  MOD_RES (5)..(5): Any amino acid or amino acid analog
XFYFX

SEQ ID NO: 137 - protein, 5 aa, Artificial Sequence
  Description of Artificial Sequence Synthetic peptide
  MOD_RES (1)..(1): Ser or Thr
XFYFP

SEQ ID NO: 138 - protein, 5 aa, Artificial Sequence
  Description of Artificial Sequence Synthetic peptide
  MOD_RES (1)..(1): Any amino acid or amino acid analog
  MOD_RES (2)..(2): Ser or Thr
XXFYF

SEQ ID NO: 139 - protein, 5 aa, Artificial Sequence
  Description of Artificial Sequence Synthetic peptide
  MOD_RES (2)..(2): Ser or Thr
PXFYF

SEQ ID NO: 140 - protein, 6 aa, Artificial Sequence
  Description of Artificial Sequence Synthetic peptide
  MOD_RES (1)..(1): Any amino acid or amino acid analog
  MOD_RES (2)..(2): Ser or Thr
  MOD_RES (6)..(6): Any amino acid or amino acid analog
XXFYFX

SEQ ID NO: 141 - protein, 6 aa, Artificial Sequence
  Description of Artificial Sequence Synthetic peptide
  MOD_RES (2)..(2): Ser or Thr
  MOD_RES (6)..(6): Any amino acid or amino acid analog
PXFYFX

SEQ ID NO: 142 - protein, 6 aa, Artificial Sequence
  Description of Artificial Sequence Synthetic peptide
  MOD_RES (1)..(1): Any amino acid or amino acid analog
  MOD_RES (2)..(2): Ser or Thr
XXFYFP

SEQ ID NO: 143 - protein, 6 aa, Artificial Sequence
  Description of Artificial Sequence Synthetic peptide
  MOD_RES (1)..(2): Any amino acid or amino acid analog
  MOD_RES (3)..(3): Ser or Thr
XXXFYF

SEQ ID NO: 144 - protein, 6 aa, Artificial Sequence
  Description of Artificial Sequence Synthetic peptide
  MOD_RES (1)..(1): Any amino acid or amino acid analog
  MOD_RES (3)..(3): Ser or Thr
XPXFYF

SEQ ID NO: 145 - protein, 6 aa, Artificial Sequence
  Description of Artificial Sequence Synthetic peptide
  MOD_RES (2)..(2): Any amino acid or amino acid analog
  MOD_RES (3)..(3): Ser or Thr
PXXFYF

SEQ ID NO: 146 - protein, 7 aa, Artificial Sequence
  Description of Artificial Sequence Synthetic peptide
  MOD_RES (1)..(2): Any amino acid or amino acid analog
  MOD_RES (3)..(3): Ser or Thr
  MOD_RES (7)..(7): Any amino acid or amino acid analog
XXXFYFX

SEQ ID NO: 147 - protein, 7 aa, Artificial Sequence
  Description of Artificial Sequence Synthetic peptide
  MOD_RES (1)..(2): Any amino acid or amino acid analog
  MOD_RES (3)..(3): Ser or Thr
XXXFYFP

SEQ ID NO: 148 - protein, 7 aa, Artificial Sequence
  Description of Artificial Sequence Synthetic peptide
  MOD_RES (2)..(2): Any amino acid or amino acid analog
  MOD_RES (3)..(3): Ser or Thr
  MOD_RES (7)..(7): Any amino acid or amino acid analog
PXXFYFX

SEQ ID NO: 149 - protein, 7 aa, Artificial Sequence
  Description of Artificial Sequence Synthetic peptide
  MOD_RES (1)..(1): Any amino acid or amino acid analog
  MOD_RES (3)..(3): Ser or Thr
  MOD_RES (7)..(7): Any amino acid or amino acid analog
XPXFYFX

SEQ ID NO: 150 - protein, 7 aa, Artificial Sequence
  Description of Artificial Sequence Synthetic peptide
  MOD_RES (1)..(1): Any amino acid or amino acid analog
  MOD_RES (3)..(3): Ser or Thr
XPXFYFP

SEQ ID NO: 151 - protein, 8 aa, Artificial Sequence
  Description of Artificial Sequence Synthetic peptide
  MOD_RES (1)..(2): Any amino acid or amino acid analog
  MOD_RES (3)..(3): Ser or Thr
  MOD_RES (7)..(8): Any amino acid or amino acid analog
XXXFYFXX

SEQ ID NO: 152 - protein, 8 aa, Artificial Sequence
  Description of Artificial Sequence Synthetic peptide
  MOD_RES (2)..(2): Any amino acid or amino acid analog
  MOD_RES (3)..(3): Ser or Thr
  MOD_RES (7)..(8): Any amino acid or amino acid analog
PXXFYFXX

SEQ ID NO: 153 - protein, 8 aa, Artificial Sequence
  Description of Artificial Sequence Synthetic peptide
  MOD_RES (1)..(1): Any amino acid or amino acid analog
  MOD_RES (3)..(3): Ser or Thr
  MOD_RES (7)..(8): Any amino acid or amino acid analog
XPXFYFXX

SEQ ID NO: 154 - protein, 8 aa, Artificial Sequence
  Description of Artificial Sequence Synthetic peptide
  MOD_RES (1)..(2): Any amino acid or amino acid analog
  MOD_RES (3)..(3): Ser or Thr
  MOD_RES (8)..(8): Any amino acid or amino acid analog
XXXFYFPX

SEQ ID NO: 155 - protein, 8 aa, Artificial Sequence
  Description of Artificial Sequence Synthetic peptide
  MOD_RES (2)..(2): Any amino acid or amino acid analog
  MOD_RES (3)..(3): Ser or Thr
  MOD_RES (8)..(8): Any amino acid or amino acid analog
PXXFYFPX

SEQ ID NO: 156 - protein, 8 aa, Artificial Sequence
  Description of Artificial Sequence Synthetic peptide
  MOD_RES (1)..(1): Any amino acid or amino acid analog
  MOD_RES (3)..(3): Ser or Thr
  MOD_RES (8)..(8): Any amino acid or amino acid analog
XPXFYFPX

SEQ ID NO: 157 - protein, 5 aa, Artificial Sequence
  Description of Artificial Sequence Synthetic peptide
  MOD_RES (2)..(2): Ser or Thr
HXFYF

SEQ ID NO: 158 - protein, 6 aa, Artificial Sequence
  Description of Artificial Sequence Synthetic peptide
  MOD_RES (2)..(2): Ser or Thr
  MOD_RES (6)..(6): Any amino acid or amino acid analog
HXFYFX

SEQ ID NO: 159 - protein, 6 aa, Artificial Sequence
  Description of Artificial Sequence Synthetic peptide
  MOD_RES (2)..(2): Any amino acid or amino acid analog
HXFYFP

SEQ ID NO: 160 - protein, 6 aa, Artificial Sequence
  Description of Artificial Sequence Synthetic peptide
  MOD_RES (1)..(1): Any amino acid or amino acid analog
  MOD_RES (3)..(3): Ser or Thr
XHXFYF

SEQ ID NO: 161 - protein, 6 aa, Artificial Sequence
  Description of Artificial Sequence Synthetic peptide
  MOD_RES (3)..(3): Ser or Thr
PHXFYF

SEQ ID NO: 162 - protein, 7 aa, Artificial Sequence
  Description of Artificial Sequence Synthetic peptide
  MOD_RES (1)..(1): Any amino acid or amino acid analog
  MOD_RES (3)..(3): Ser or Thr
  MOD_RES (7)..(7): Any amino acid or amino acid analog
XHXFYFX

SEQ ID NO: 163 - protein, 7 aa, Artificial Sequence
  Description of Artificial Sequence Synthetic peptide
  MOD_RES (1)..(1): Any amino acid or amino acid analog
  MOD_RES (3)..(3): Ser or Thr
XHXFYFP

SEQ ID NO: 164 - protein, 7 aa, Artificial Sequence
  Description of Artificial Sequence Synthetic peptide
  MOD_RES (3)..(3): Ser or Thr
  MOD_RES (7)..(7): Any amino acid or amino acid analog
PHXFYFX

SEQ ID NO: 165 - protein, 7 aa, Artificial Sequence
  Description of Artificial Sequence Synthetic peptide
  MOD_RES (3)..(3): Ser or Thr
PHXFYFP

SEQ ID NO: 166 - protein, 8 aa, Artificial Sequence
  Description of Artificial Sequence Synthetic peptide
  MOD_RES (1)..(1): Any amino acid or amino acid analog
  MOD_RES (3)..(3): Ser or Thr
  MOD_RES (7)..(8): Any amino acid or amino acid analog
XHXFYFXX

SEQ ID NO: 167 - protein, 8 aa, Artificial Sequence
  Description of Artificial Sequence Synthetic peptide
  MOD_RES (3)..(3): Ser or Thr
  MOD_RES (7)..(8): Any amino acid or amino acid analog
PHXFYFXX

SEQ ID NO: 168 - protein, 8 aa, Artificial Sequence
  Description of Artificial Sequence Synthetic peptide
  MOD_RES (1)..(1): Any amino acid or amino acid analog
  MOD_RES (3)..(3): Ser or Thr
  MOD_RES (8)..(8): Any amino acid or amino acid analog
XHXFYFPX

SEQ ID NO: 169 - protein, 8 aa, Artificial Sequence
  Description of Artificial Sequence Synthetic peptide
  MOD_RES (3)..(3): Ser or Thr
  MOD_RES (8)..(8): Any amino acid or amino acid analog
PHXFYFPX

SEQ ID NO: 170 - protein, 3 aa, Artificial Sequence
  Description of Artificial Sequence Synthetic peptide
FYY

SEQ ID NO: 171 - protein, 4 aa, Artificial Sequence
  Description of Artificial Sequence Synthetic peptide
FYYP

SEQ ID NO: 172 - protein, 5 aa, Artificial Sequence
  Description of Artificial Sequence Synthetic peptide
PFYYP

SEQ ID NO: 173 - protein, 6 aa, Artificial Sequence
  Description of Artificial Sequence Synthetic peptide
  MOD_RES (2)..(2): Any amino acid or amino acid analog
PXFYYP

SEQ ID NO: 174 - protein, 6 aa, Artificial Sequence
  Description of Artificial Sequence Synthetic peptide
PSFYYP

SEQ ID NO: 175 - protein, 6 aa, Artificial Sequence
  Description of Artificial Sequence Synthetic peptide
PTFYYP

SEQ ID NO: 176 - protein, 5 aa, Artificial Sequence
  Description of Artificial Sequence Synthetic peptide
TFYFP

SEQ ID NO: 177 - protein, 5 aa, Artificial Sequence
  Description of Artificial Sequence Synthetic peptide
SFYFP

SEQ ID NO: 178 - protein, 8 aa, Artificial Sequence
  Description of Artificial Sequence Synthetic peptide
  MOD_RES (1)..(3): Any amino acid or amino acid analog
  MOD_RES (7)..(8): Any amino acid or amino acid analog
XXXFYFXX

SEQ ID NO: 179 - protein, 15 aa, Artificial Sequence
  Description of Artificial Sequence Synthetic peptide
AGEDPHGFYFPGQFA

SEQ ID NO: 180 - protein, 22 aa, Artificial Sequence
  Description of Artificial Sequence Synthetic peptide
RRRRRRRAGEDPHSFYFPGQFA

SEQ ID NO: 181 - protein, 7 aa, Artificial Sequence
  Description of Artificial Sequence Synthetic peptide
RRRRRRR

SEQ ID NO: 182 - protein, 15 aa, Artificial Sequence
  Description of Artificial Sequence Synthetic peptide
  MOD_RES (1)..(15): D-amino acid
AFQGPFYFSHPDEGA

SEQ ID NO: 183 - protein, 6 aa, Artificial Sequence
  Description of Artificial Sequence Synthetic peptide
  MOD_RES (3)..(3): phospho-Thr
KRTIRR

SEQ ID NO: 184 - protein, 11 aa, Artificial Sequence
  Description of Artificial Sequence Synthetic peptide
  MOD_RES (1)..(4): Any amino acid
  MOD_RES (8)..(11): Any amino acid
XXXXFYFXXXX

SEQ ID NO: 185 - protein, 11 aa, Artificial Sequence
  Description of Artificial Sequence Synthetic peptide
  MOD_RES (1)..(4): Any amino acid
  MOD_RES (8)..(11): Any amino acid
XXXXFYAXXXX

SEQ ID NO: 186 - DNA, 10 bp, Artificial Sequence
  Description of Artificial Sequence Synthetic oligonucleotide
  modified_base (1)..(5): a, c, t, g, unknown or other
  modified_base (6)..(6): Unnatural hydrophobic nucleic acid
  modified_base (7)..(10): a, c, t, g, unknown or other
nnnnnnnnnn

SEQ ID NO: 187 - DNA, 10 bp, Artificial Sequence
  Description of Artificial Sequence Synthetic oligonucleotide
  modified_base (1)..(10): a, c, t, g, unknown or other
nnnnnnnnnn

SEQ ID NO: 188 - protein, 2 aa, Artificial Sequence
  Description of Artificial Sequence Synthetic peptide
  MOD_RES (2)..(2): Any amino acid
GX

SEQ ID NO: 189 - protein, 2 aa, Artificial Sequence
  Description of Artificial Sequence Synthetic peptide
  MOD_RES (1)..(1): Any amino acid
XP

SEQ ID NO: 190 - protein, 2 aa, Artificial Sequence
  Description of Artificial Sequence Synthetic peptide
  MOD_RES (1)..(1): Any amino acid
XS

SEQ ID NO: 191 - protein, 2 aa, Artificial Sequence
  Description of Artificial Sequence Synthetic peptide
  MOD_RES (2)..(2): Any amino acid
DX

SEQ ID NO: 192 - protein, 2 aa, Artificial Sequence
  Description of Artificial Sequence Synthetic peptide
  MOD_RES (2)..(2): Any amino acid
PX

SEQ ID NO: 193 - protein, 2 aa, Artificial Sequence
  Description of Artificial Sequence Synthetic peptide
  MOD_RES (1)..(1): Any amino acid
XG

SEQ ID NO: 194 - protein, 14 aa, Artificial Sequence
  Description of Artificial Sequence Synthetic peptide
AGEDPHSFYFPGQF

SEQ ID NO: 195 - protein, 14 aa, Artificial Sequence
  Description of Artificial Sequence Synthetic peptide
AGEDPGYFLPGQFA

SEQ ID NO: 196 - protein, 14 aa, Artificial Sequence
  Description of Artificial Sequence Synthetic peptide
AGEDPSFYFPGQFA

SEQ ID NO: 197 - protein, 15 aa, Artificial Sequence
  Description of Artificial Sequence Synthetic peptide
AGEDPSFYFPGQFAF

SEQ ID NO: 198 - protein, 16 aa, Artificial Sequence
  Description of Artificial Sequence Synthetic peptide
AGEDPSFYFPGQFAFS

SEQ ID NO: 199 - protein, 14 aa, Artificial Sequence
  Description of Artificial Sequence Synthetic peptide
  MOD_RES (1)..(1): Any amino acid or absent
XGEDPSFYFPGQFA

SEQ ID NO: 200 - protein, 14 aa, Artificial Sequence
  Description of Artificial Sequence Synthetic peptide
  MOD_RES (2)..(2): Any amino acid or absent
AXEDPSFYFPGQFA

SEQ ID NO: 201 - protein, 14 aa, Artificial Sequence
  Description of Artificial Sequence Synthetic peptide
  MOD_RES (3)..(3): Any amino acid or absent
AGXDPSFYFPGQFA

SEQ ID NO: 202 - protein, 14 aa, Artificial Sequence
  Description of Artificial Sequence Synthetic peptide
  MOD_RES (4)..(4): Any amino acid or absent
AGEXPSFYFPGQFA

SEQ ID NO: 203 - protein, 14 aa, Artificial Sequence
  Description of Artificial Sequence Synthetic peptide
  MOD_RES (5)..(5): Any amino acid or absent
AGEDXSFYFPGQFA

SEQ ID NO: 204 - protein, 14 aa, Artificial Sequence
  Description of Artificial Sequence Synthetic peptide
  MOD_RES (6)..(6): Any amino acid or absent
AGEDPXFYFPGQFA

SEQ ID NO: 205 - protein, 14 aa, Artificial Sequence
  Description of Artificial Sequence Synthetic peptide
  MOD_RES (7)..(7): Any amino acid or absent
AGEDPSXYFPGQFA

SEQ ID NO: 206 - protein, 14 aa, Artificial Sequence
  Description of Artificial Sequence Synthetic peptide
  MOD_RES (8)..(8): Any amino acid or absent
AGEDPSFXFPGQFA

SEQ ID NO: 207 - protein, 14 aa, Artificial Sequence
  Description of Artificial Sequence Synthetic peptide
  MOD_RES (9)..(9): Any amino acid or absent
AGEDPSFYXPGQFA

SEQ ID NO: 208 - protein, 14 aa, Artificial Sequence
  Description of Artificial Sequence Synthetic peptide
  MOD_RES (10)..(10): Any amino acid or absent
AGEDPSFYFXGQFA

SEQ ID NO: 209 - protein, 14 aa, Artificial Sequence
  Description of Artificial Sequence Synthetic peptide
  MOD_RES (11)..(11): Any amino acid or absent
AGEDPSFYFPXQFA

SEQ ID NO: 210 - protein, 14 aa, Artificial Sequence
  Description of Artificial Sequence Synthetic peptide
  MOD_RES (12)..(12): Any amino acid or absent
AGEDPSFYFPGXFA

SEQ ID NO: 211 - protein, 14 aa, Artificial Sequence
  Description of Artificial Sequence Synthetic peptide
  MOD_RES (13)..(13): Any amino acid or absent
AGEDPSFYFPGQXA

SEQ ID NO: 212 - protein, 14 aa, Artificial Sequence
  Description of Artificial Sequence Synthetic peptide
  MOD_RES (14)..(14): Any amino acid or absent
AGEDPSFYFPGQFX

SEQ ID NO: 213 - protein, 13 aa, Artificial Sequence
  Description of Artificial Sequence Synthetic peptide
  MOD_RES (7)..(7): Any amino acid or absent
  MOD_RES (9)..(9): Any amino acid or absent
AGEDPSXYXGQFA

SEQ ID NO: 214 - protein, 12 aa, Artificial Sequence
  Description of Artificial Sequence Synthetic peptide
  MOD_RES (7)..(8): Any amino acid or absent
AGEDPSXXGQFA

SEQ ID NO: 215 - protein, 14 aa, Artificial Sequence
  Description of Artificial Sequence Synthetic peptide
  MOD_RES (1)..(1): Any unnatural amino acid
XGEDPSFYFPGQFA

SEQ ID NO: 216 - protein, 14 aa, Artificial Sequence
  Description of Artificial Sequence Synthetic peptide
  MOD_RES (2)..(2): Any unnatural amino acid
AXEDPSFYFPGQFA

SEQ ID NO: 217 - protein, 14 aa, Artificial Sequence
  Description of Artificial Sequence Synthetic peptide
  MOD_RES (3)..(3): Any unnatural amino acid
AGXDPSFYFPGQFA

SEQ ID NO: 218 - protein, 14 aa, Artificial Sequence
  Description of Artificial Sequence Synthetic peptide
  MOD_RES (4)..(4): Any unnatural amino acid
AGEXPSFYFPGQFA

SEQ ID NO: 219 - protein, 14 aa, Artificial Sequence
  Description of Artificial Sequence Synthetic peptide
  MOD_RES (5)..(5): Any unnatural amino acid
AGEDXSFYFPGQFA

SEQ ID NO: 220 - protein, 14 aa, Artificial Sequence
  Description of Artificial Sequence Synthetic peptide
  MOD_RES (6)..(6): Any unnatural amino acid
AGEDPXFYFPGQFA

SEQ ID NO: 221 - protein, 14 aa, Artificial Sequence
  Description of Artificial Sequence Synthetic peptide
  MOD_RES (7)..(7): Any unnatural amino acid
AGEDPSXYFPGQFA

SEQ ID NO: 222 - protein, 14 aa, Artificial Sequence
  Description of Artificial Sequence Synthetic peptide
  MOD_RES (8)..(8): Any unnatural amino acid
AGEDPSFXFPGQFA

SEQ ID NO: 223 - protein, 14 aa, Artificial Sequence
  Description of Artificial Sequence Synthetic peptide
  MOD_RES (9)..(9): Any unnatural amino acid
AGEDPSFYXPGQFA

SEQ ID NO: 224 - protein, 14 aa, Artificial Sequence
  Description of Artificial Sequence Synthetic peptide
  MOD_RES (10)..(10): Any unnatural amino acid
AGEDPSFYFXGQFA

SEQ ID NO: 225 - protein, 14 aa, Artificial Sequence
  Description of Artificial Sequence Synthetic peptide
  MOD_RES (11)..(11): Any unnatural amino acid
AGEDPSFYFPXQFA

SEQ ID NO: 226 - protein, 14 aa, Artificial Sequence
  Description of Artificial Sequence Synthetic peptide
  MOD_RES (12)..(12): Any unnatural amino acid
AGEDPSFYFPGXFA

SEQ ID NO: 227 - protein, 14 aa, Artificial Sequence
  Description of Artificial Sequence Synthetic peptide
  MOD_RES (13)..(13): Any unnatural amino acid
AGEDPSFYFPGQXA

SEQ ID NO: 228 - protein, 14 aa, Artificial Sequence
  Description of Artificial Sequence Synthetic peptide
  MOD_RES (14)..(14): Any unnatural amino acid
AGEDPSFYFPGQFX

SEQ ID NO: 229 - protein, 13 aa, Artificial Sequence
  Description of Artificial Sequence Synthetic peptide
  MOD_RES (7)..(7): Any unnatural amino acid
  MOD_RES (9)..(9): Any unnatural amino acid
AGEDPSXYXGQFA

SEQ ID NO: 230 - protein, 12 aa, Artificial Sequence
  Description of Artificial Sequence Synthetic peptide
  MOD_RES (7)..(8): Any unnatural amino acid
AGEDPSXXGQFA

SEQ ID NO: 231 - protein, 6 aa, Artificial Sequence
  Description of Artificial Sequence Synthetic peptide
RRRRRR

SEQ ID NO: 232 - protein, 52 aa, Unknown
  Description of Unknown PP2A regulatory subunit B-gamma polypeptide
MGEDTDTRKINHSFLRDHSYVTEADVISTVEFNHTGELLATGDKGGRVVIFQ

SEQ ID NO: 233 - protein, 67 aa, Unknown
  Description of Unknown PP2A regulatory subunit B-gamma polypeptide
REPESKNAPHSQGEYDVYSTFQSHEPEFDYLKSLEIEEKINKIKWLPQQNAAHSLLSTNDKTIKLWK

SEQ ID NO: 234 - protein, 81 aa, Unknown
  Description of Unknown PP2A regulatory subunit B-gamma polypeptide
ITEDNKRPEGYNLKDEEGKLKDLSTVTSLQVPVLKPMDLMVEVSPRRIFANGHTYHINSISVNSDCETYMSADDLRINLWH

SEQ ID NO: 235 - protein, 51 aa, Unknown
  Description of Unknown PP2A regulatory subunit B-gamma polypeptide
LAITDRSFNIVDIKPANMEDLTEVITASEFHPHHCNLFVYSSSKGSLRLCD

SEQ ID NO: 236 - protein, 57 aa, Unknown
  Description of Unknown PP2A regulatory subunit B-gamma polypeptide
MRAAALCDKHSKLFEEPEDPSNRSFFSEIISSVSDVKFSHSGRYMLTRDYLTVKVWD

SEQ ID NO: 237 - protein, 58 aa, Unknown
  Description of Unknown PP2A regulatory subunit B-gamma polypeptide
LNMEARPIETYQVHDYLRSKLCSLYESDCIFDKFECAWNGSDSVIMTGAYNNFFRMFD

SEQ ID NO: 238 - protein, 81 aa, Unknown
  Description of Unknown PP2A regulatory subunit B-gamma polypeptide
RNTKRDVTLEASRESSKPRAVLKPRRVCVGGKRRRDDISVDSLDFTKKILHTAWHPAENIIAIAATNNLYIFQDKVNSDMH

SEQ ID NO: 239 - protein, 11 aa, Homo sapiens
IPTFYFPRGRP

SEQ ID NO: 240 - protein, 11 aa, Homo sapiens
IPKFYFPKGCP

SEQ ID NO: 241 - protein, 11 aa, Mus musculus
VPAFYFPCGRP

SEQ ID NO: 242 - protein, 11 aa, Xenopus sp.
IPRFYFPEGLP

SEQ ID NO: 243 - protein, 11 aa, Drosophila sp.
IPRFYFPHGKP

SEQ ID NO: 244 - protein, 11 aa, Homo sapiens
EPEFDYLKSLE

SEQ ID NO: 245 - protein, 11 aa, Rattus sp.
EPEFDYLKSLE

SEQ ID NO: 246 - protein, 10 aa, Homo sapiens
CILFDFMDSV

SEQ ID NO: 247 - protein, 10 aa, Homo sapiens
GVMFDFLDCV

SEQ ID NO: 248 - protein, 11 aa, Homo sapiens
CVLFDFVSDPL

SEQ ID NO: 249 - protein, 10 aa, Homo sapiens
CVIFDFMDTL

SEQ ID NO: 250 - protein, 11 aa, Homo sapiens
IPRFYFGEGLP

SEQ ID NO: 251 - protein, 11 aa, Xenopus sp.
IPKFYFPKGCP

SEQ ID NO: 252 - protein, 11 aa, Homo sapiens
IPTFYFPRGRP

SEQ ID NO: 253 - protein, 11 aa, Artificial Sequence
  Description of Artificial Sequence Synthetic peptide
IPTAYFPRGRP

SEQ ID NO: 254 - protein, 11 aa, Artificial Sequence
  Description of Artificial Sequence Synthetic peptide
IPTFAFPRGRP

SEQ ID NO: 255 - protein, 11 aa, Artificial Sequence
  Description of Artificial Sequence Synthetic peptide
IPTFYAPRGRP

SEQ ID NO: 256 - protein, 11 aa, Artificial Sequence
  Description of Artificial Sequence Synthetic peptide
IPTAYAPRGRP

SEQ ID NO: 257 - protein, 11 aa, Artificial Sequence
  Description of Artificial Sequence Synthetic peptide
IPTAAAPRGRP

SEQ ID NO: 258 - protein, 3 aa, Artificial Sequence
  Description of Artificial Sequence Synthetic peptide
AYF

SEQ ID NO: 259 - protein, 3 aa, Artificial Sequence
  Description of Artificial Sequence Synthetic peptide
FAF

SEQ ID NO: 260 - protein, 3 aa, Artificial Sequence
  Description of Artificial Sequence Synthetic peptide
FYA

SEQ ID NO: 261 - protein, 3 aa, Artificial Sequence
  Description of Artificial Sequence Synthetic peptide
AYA

SEQ ID NO: 262 - protein, 3 aa, Artificial Sequence
  Description of Artificial Sequence Synthetic peptide
AAA

SEQ ID NO: 263 - protein, 17 aa, Artificial Sequence
  Description of Artificial Sequence Synthetic peptide
  MOD_RES (6)..(9): Any amino acid
AGEDPXXXXFPGQFAFS

SEQ ID NO: 264 - protein, 575 aa, Homo sapiens
MPPGKVLQPVLKMKVDELFLYWLSEASTQRMLQDCLRRIKAPGRDQPTPGDGEQPGAWPTAPLAAPRPSGLEPPGTPGPGPALPLGAASSPRNAPHVRGTRRSAGTRVVQTRKEEPLPPATSQSIPTFYFPRGRPQDSVNVDAVISKIESTFARFPHERATMDDMGLVAKACGCPLYWKGPLFYGAGGERTGSVSVHKFVAMWRKILQNCHDDAAKFVHLLMSPGCNYLVQEDFVPFLQDVVNTHPGLSFLKEASEFHSRYITTVIQRIFYAVNRSWSGRITCAELRRSSFLQNVALLEEEADINQLTEFFSYEHFYVIYCKFWELDTDHDLLIDADDLARHNDHALSTKMIDRIFSGAVTRGRKVQKEGKISYADFVWFLISEEDKKTPTSIEYWFRCMDLDGDGALSMFELEYFYEEQCRRLDSMAIEALPFQDCLCQMLDLVKPRTEGKITLQDLKRCKLANVFFDTFFNIEKYLDHEQKEQISLLRDGDSGGPELSDWEKYAAEEYDILVAEETAGEPWEDGFEAELSPVEQKLSALRSPLAQRPFFEAPSPLGAVDLYEYACGDEDLEPL

SEQ ID NO: 265 - protein, 529 aa, Homo sapiens
MMIKETSLRRDPDLRGELAFLARGCDFVLPSRFKKRLKSFQQTQIQNKPEKKPGTPLPPPATSPSSPRPLSPVPHVNNVVNAPLSINIPRFYFPEGLPDTCSNHEQTLSRIETAFMDIEEQKADIYEMGKIAKVCGCPLYWKAPMFRAAGGEKTGFVTAQSFIAMWRKLLNNHHDDASKFICLLAKPNCSSLEQEDFIPLLQDVVDTHPGLTFLKDAPEFHSRYITTVIQRIFYTVNRSWSGKITSTEIRKSNFLQTLALLEEEEDINQITDYFSYEHFYVIYCKFWELDTDHDLYISQADLSRYNDQASSSRIIERIFSGAVTRGKTIQKEGRMSYADFVWFLISEEDKRNPTSIEYWFRCMDVDGDGVLSMYELEYFYEEQCERMEAMGIEPLPFHDLLCQMLDLVKPAVDGKITLRDLKRCRMAHIFYDTFFNLEKYLDHEQRDPFAVQKDVENDGPEPSDWDRFAAEEYETLVAEESAQAQFQEGFEDYETDEPASPSEFGNKSNKILSASLPEKCGKLQSVDEE

SEQ ID NO: 266 - protein, 6 aa, Artificial Sequence
  Description of Artificial Sequence Synthetic 6xHis tag
HHHHHH